(19) United States
(12) Patent Application Publication
(10) Pub. No.: US 2005/0059615 A1
(43) Pub. Date: Mar. 17, 2005

(54) Transmucosal dosage forms for brain-targeted steroid chemical delivery systems

(76) Inventor: Nicholas S. Bodor, Gainesville, FL (US)

Correspondence Address: IVAX CORPORATION, 4400 BISCAYNE BOULEVARD, MIAMI, FL, 33137, US

(21) Appl. No.: 10/910,965

(22) Filed: Aug. 2, 2004

Related U.S. Application Data
(60) Provisional application No. 60/491,233, filed on Jul. 31, 2003.
(60) Provisional application No. 60/491,234, filed on Jul. 31, 2003.
(60) Provisional application No. 60/586,506, filed on Jul. 9, 2004.

(51) Int. Cl. A61K 31/724; A61K 31/704

(52) U.S. Cl. 514/26; 514/58

(57) ABSTRACT

Provided are pharmaceutical compositions of an essentially saturated complex of a steroidal chemical delivery system with cyclodextrin, formulated into a transmucosal dosage form, and methods for their use.

### CROSS-REFERENCE TO RELATED APPLICATIONS

This application claims the priority of U.S. Provisional Applications No. 60/491,234, filed Jul. 31, 2003, Ser. No. 60/491,233, filed Jul. 31, 2003 and Ser. No. 60/586,506, filed Jul. 9, 2004 (Attorney Docket No. IVAX0024-P-USA, entitled “TRANSMUCOSAL DOSAGE FORMS FOR BRAIN-TRAGETED STEROID CHEMICAL DELIVERY SYSTEMS”), each of which is incorporated by reference herein in its entirety and relied upon.

### FIELD OF THE INVENTION

The invention relates to a cyclodextrin complex of a chemical delivery system for steroids, formulated into a transmucosal dosage form, and to a method for enhancing the transmucosal bioavailability of the chemical delivery system.

### BACKGROUND OF THE INVENTION

A brain-targeted chemical delivery system (CDS) represents a rational drug design approach which exploits sequential metabolism, not only to deliver but also to target drugs to their site of action. A dihydropyridine [symbol] pyridinium salt-type redox system has been previously proposed and applied to a number of drugs, including steroidal sex hormones such as estradiol and testosterone and anti-inflammatory steroids such as dexamethasone. According to this redox system, a centrally acting drug [D] is coupled to a quaternary carrier [QC]⁺ through a reactive functional group (such as a hydroxyl function) in the drug; the [D-QC]⁺ which results is then reduced chemically to the lipoidal dihydro form [D-DHC]. After administration of [D-DHC] in vivo, it is rapidly distributed throughout the body, including the brain. The dihydro form [D-DHC] is then in situ oxidized (by the NAD [symbol] NADH system) to the ideally inactive original [D-QC]⁺ quaternary salt which, because of its ionic, hydrophilic character, is rapidly eliminated from the general circulation of the body, while the blood-brain barrier prevents its elimination from the brain. Enzymatic change of the [D-QC]⁺ which is “locked” in the brain effects a sustained delivery of the drug species [D], followed by its normal elimination. A properly selected carrier [QC]⁺ will also be rapidly eliminated from the brain. Because of the facile elimination of [D-QC]⁺ from the general circulation, only minor amounts of the drug [D] will be released in the brain. The overall result will be a brain-specific sustained release of the target drug species. See, for example, Bodor U.S. Pat. Nos. 4,479,932; 4,540,564; 4,880,921; and 4,900,837. The compound 17β-[(1-methyl-1,4-dihydro-3-pyridinyl)carbonyloxy]estra-1,3,5(10)-trien-3-ol, which has the structure 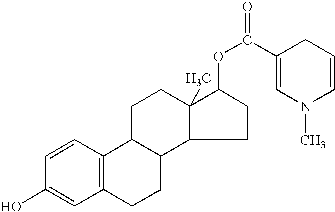
and is also known as E₂-CDS, is a specific CDS devised for estradiol which is described in these patents. In this case, the lipophilic 17-dihydrotrigonelline ester of estradiol, i.e. E²-CDS, is enzymatically converted to the hydrophilic trigonellinate ester (E₂-Q⁺), which is specifically retained in the brain due to the characteristics of the BBB. The hydrophilic (E₂-Q⁺) form is thus “locked” in the brain and is slowly and sustainedly hydrolyzed by esterases to estradiol (E₂). Similar E₂-CDS→E₂Q⁺ conversion in the rest of the body accelerates peripheral elimination and improves targeting.

The dihydropyridine [symbol] pyridinium salt redox carrier system achieved remarkable success in targeting drugs to the brain in laboratory tests. This success was, of course, due in part to the highly lipophilic nature of the dihydropyridine-containing derivatives, which allows brain penetration. At the same time, the increased lipophilicity makes it practically impossible to formulate aqueous solutions of these derivatives for injection; moreover, even in organic solvents such as DMSO, they have a propensity for precipitating out of solution upon injection, particularly at higher concentrations and especially at the injection site or in the lungs. Even in the absence of noticeable crystallization, the redox derivatives frequently display not only the desired concentration in the brain but undesired initial high lung concentrations as well. Further, the dihydropyridine-containing derivatives suffer from stability problems, since even in the dry state they are very sensitive to oxidation as well as to water addition.

Cyclodextrins are cyclic oligosaccharides composed of cyclic α-(1→4) linked D-glucopyranose units. Cyclodextrins with six to eight units have been named α-, β- and γ-cyclodextrin, respectively. The number of units determines the size of the cone-shaped cavity which characterizes cyclodextrins and into which drugs may include and form stable complexes. A number of derivatives of α-, β- and γ-cyclodextrin are known in which one or more hydroxyl groups is/are replaced with ether groups or other radicals. These compounds are thus known complexing agents and have been previously used in the pharmaceutical field to form inclusion complexes with water-insoluble drugs and to thus solubilize them in aqueous media.

Cyclodextrin complexation was found to offer a solution to the various problems with the redox derivatives discussed above. These problems are addressed in Bodor U.S. Pat. Nos. 5,002,935; 5,017,566; 4,983,586; and 5,024,998. In particular, U.S. Pat. Nos. 5,002,935 and 5,017,566 describe inclusion complexes of hydroxypropyl, hydroxyethyl, glucosyl, maltosyl and maltotriosyl derivatives of β- and γ-cyclodextrin with the reduced, biooxidizable, blood-brain barrier penetrating, lipoidal forms of dihydropyridine [symbol] pyridinium salt redox systems for brain-targeted drug delivery which provide a means for stabilizing the redox systems, particularly against oxidation. The redox inclusion complexes also provide a means for decreasing initial drug concentrations in the lungs after administration of the systems, leading to decreased toxicity. In selected instances, complexation results in substantially improved water solubility of the redox systems as well. Contemplated routes of administration for the complexes are said to include oral, buccal, sublingual, topical (including ophthalmic), rectal, vaginal, nasal and parenteral (including intravenous, intramuscular and subcutaneous). However, no specific formulations are disclosed in the patents except in the case of parenteral administration. Specific complexes with E₂-CDS are described and illustrated in detail. FIG. 1 in both the '935 and '566 patents is a phase solubility diagram illustrating the increase in solubility of E₂-CDS with increasing concentrations of hydroxypropyl-β-cyclodextrin (HPβCD) in water. The straight line indicates the formation of a 1:1 complex. See also Brewster et al., Journal of Pharmaceutical Sciences, Vol. 77, No. 11, November 1988, 981-985, which describes work with E₂-CDS and a number of different cyclodextrins, and FIG. 1 therein which also appears to show formation of a 1:2 complex at high HPβCD concentrations. In addition, see the report on preliminary work on use of carboxymethylethyl-β-cyclodextrin with E₂-CDS for possible oral use by Brewster et al., “Improved Oral Bioavailability of the Brain-Targeting Estrogen, E₂-CDS, Through the Use of Carboxymethylethyl-β-Cyclodextrin”, Proceedings of the 8^{th} International Cyclodextrin Symposium, Budapest (Hungary): Mar. 30 to Apr. 2, 1996, Editions de Sante, Paris.

The most studied of the dihydropyridine redox carrier drugs appears to be the aforementioned delivery system for estradiol, E₂-CDS. E₂-CDS has been previously suggested for a number of uses, including treatment of male sexual dysfunction (Anderson et al. U.S. Pat. No. 4,863,911) and weight control (Bodor et al. U.S. Pat. No. 4,617,298), as well as brain-specific, steroid deprivation syndromes (such as hot flushes) and for chronic reduction of gonadotropin secretion for fertility regulation (contraception) or treatment of gonadal steroid-dependent diseases, such as endometriosis and prostatic hypertrophy (noted in column 46 of Bodor et al. U.S. Pat. No. 4,617,298). These prior investigations focused on the ability of E₂-CDS to produce extremely long-acting effects (of the order of 1 month) from a single selected dose of E₂-CDS. This was considered highly desirable for dosing purposes, as once-a-month administration was deemed particularly convenient, especially for purposes of contraception. Initially high levels of peripheral estrogen were not a concern, as they were lower than the levels produced by equimolar conventional estrogen, and LH reduction was comparable to that obtained with equimolar conventional estrogen. In rats, 3 mg/kg of E₂-CDS was typically used to provide activity for a period of 1 month. Such an amount is generally 10 times the mg/kg amount expected to be comparable in humans. Thus, a 0.3 mg/kg amount was expected to provide comparable results in women. For an overview of prior work with E₂-CDS, see Brewster et al., Rev. Neurosci. 2, 241-285 (1990), who also suggest that buccal administration in humans may ultimately be feasible. See also Brewster et al., J. Pharm Sci. 77: 981-985 (1988); Estes et al., Life Sciences 40, 1327-1334 (1987); Anderson et al. Life Sciences 42, 1493-1502 (1988); and Rabimy et al., Maturitas 13, 51-63 (1991).

Lower doses were also included as part of toxicity testing in women, as is required to establish safety. However, it was expected that dosages would need to be about 0.3 mg/kg in postmenopausal women to effectively suppress LH and treat postmenopausal symptoms long term.

In the male, too, prior investigations focused on the ability of E₂-CDS to produce extremely long-acting effects (of the order of 1 month) from a single selected dose of E₂-CDS. This was considered highly desirable for dosing purposes, as once-a-month administration was deemed particularly convenient. The aforementioned Anderson et al. patent relating to male sexual dysfunction showed that, in castrated male rats, an amount of E₂-CDS of 3 mg/kg i.v. was typically used to provide activity for a period of 1 month. This amount stimulated mounting behavior, increased intromission behavior and reduced both mount latency and intromission latency. The conclusion was that E₂-CDS was a potent, long-acting stimulant of the proceptive components of masculine sexual behavior. The Anderson et al. patent suggested use of E₂-CDS alone if deficits in peripheral androgen-responsive tissues were not an issue; in other cases, administration together with an androgen such as testosterone was suggested. Such an amount of 3 mg/kg is generally 10 times the mg/kg amount expected to be comparable in humans. Thus, a 0.3 mg/kg amount was expected to provide comparable results in men. See also Brewster et al., Rev. Neurosci. 2, 241-285 (1990).

Given the duration of action of E₂-CDS, it was thought that once-monthly dosing in humans, for example in parenteral or even buccal formulations of its complex with an appropriate cyclodextrin such as hydroxypropyl-β-cyclodextrin, would not be impractical.

Recently, however, the generally accepted notion that treatment of postmenopausal women with estrogen combined with progestin offered protection from coronary heart disease as well as improvement in health-related quality of life has not proved to be correct. Constant elevated peripheral exposure to estrogens may in fact lead to a number of pathological conditions, including breast cancer, coronary heart disease and pulmonary embolism; Beral et al., Lancet, 360 (9337), 942-944 (2002). Contrary to earlier expectations, hormone replacement therapy (HRT) does not lower the incidence of coronary heart disease; Low et al., Am. J. Med. Sci., 324(4), 180-184 (2002). The estrogen plus progestin combination of the Women's Health Initiative trial in postmenopausal women was stopped prematurely due to an unacceptably increased risk for invasive breast cancer, stroke and heart attack; Rossouw et al., J. Am. Med. Assoc. 288, 321-333 (2002).

Recent studies have also shown that elevated peripheral exposure to estrogen is generally undesirable in males.

It is thus apparent that E₂-CDS cannot realize its full potential until it can be delivered in a way which will still achieve its therapeutic function but will not significantly elevate peripheral exposure to estrogen. Of course, the situation would be expected to be comparable for the chemical delivery systems of other sex hormones, be they estrogens, progestins or androgens, that is, that they would be more useful if they could be delivered in effective amounts which maintain acceptably low peripheral hormone levels. The same may be said of chemical delivery systems for anti-inflammatory steroids. While literature reports show that administration of the chemical delivery systems provide much lower peripheral hormone levels than the steroids from which they are derived, the reported peripheral levels are significant and higher than would be desirable.

Oral and transmucosal delivery of drugs is often preferred to parenteral delivery for a variety of reasons, foremost patient compliance, or for cost or therapeutic considerations. Patient compliance is enhanced insofar as oral and transmucosal dosage forms alleviate repeated health care provider visits, or the discomfort of injections or prolonged infusion times associated with some active drugs. At a time of escalating health care costs, the reduced costs associated with oral or transmucosal administration versus parenteral administration costs gain importance. The cost of parenteral administration is much higher due to the requirement that a health care professional administer the drug in the health care provider setting, which also includes all attendant costs associated with such administration. Furthermore, in the present case, therapeutic consideration of the need to avoid significantly elevating peripheral steroid levels over a prolonged period of time may be practically met only by oral or transmucosal delivery. Oral delivery is, however, not practical for E₂-CDS or the other dihydropyridine redox carrier compounds; the dihydrotrigonellinate moiety in E₂-CDS, for example, shows instability in gastrointestinal fluid leading to multiple decomposition products starting with water addition and/or oxidation. Transmucosal delivery, on the other hand, has never been optimized for these drugs. In particular, the art has not suggested dosage forms and/or dosing regimens particularly adapted for transmucosal administration of E₂-CDS and other steroid-CDS drugs, that is, forms and regimens specially intended for administration through the mucosa lining the nasal, oral, vaginal or rectal cavities rather than via the orogastric route, for achieving the desired therapeutic effects possible from parenteral administration while still maintaining acceptably low peripheral steroid levels. As noted earlier, buccal administration has been previously suggested for the inclusion complexes of the steroid-CDS drugs, and the E₂-CDS complex with hydroxypropyl-β-cyclodextrin (HPβCD) has in fact been previously formulated for buccal administration in clinical trials, but neither the buccal forms nor the buccal regimens previously described for the E₂-CDS/HPβCD complex have achieved the desired therapeutic effects while still maintaining acceptably low peripheral steroid levels. Further, the art does not suggest how to maximize or enhance the benefits of complexation in terms of bioavailability and interpatient variation when the complex is to be administered as a transmucosal dosage form.

### SUMMARY OF THE INVENTION

It is now believed that excess cyclodextrin inhibits the absorption of chemical delivery systems for steroidal sex hormones or anti-inflammatory steroids (S-CDS) from a transmucosal dosage form comprising an S-CDS-cyclodextrin complex, and that a transmucosal dosage form of a saturated S-CDS-cyclodextrin complex improves oral and/or transmucosal bioavailability and/or achieves lower interpatient and/or intrapatient variation of the S-CDS and/or maintains acceptably low peripheral steroid levels.

The present invention provides a pharmaceutical composition comprising an essentially saturated S-CDS-cyclodextrin complex formulated into a transmucosal dosage form which is substantially free of cyclodextrin in excess of the minimum amount needed to maximize the amount of S-CDS in the complex, the amount of S-CDS in the complex being an amount effective to elicit a therapeutic response while maintaining acceptably low peripheral steroid levels. In a particular aspect of the invention, the pharmaceutical composition comprises an essentially saturated S-CDS-cyclodextrin complex formulated into a transmucosal dosage form which is substantially free of cyclodextrin in excess of the minimum amount needed to maintain substantially all of the S-CDS in the complex. This composition provides the S-CDS in its highest thermodynamic activity state at the time it contacts the rectal, vaginal, buccal or nasal mucosa.

The invention also provides a method for increasing the transmucosal bioavailability of the S-CDS comprising administering to a subject in need thereof, a pharmaceutical composition comprising an essentially saturated S-CDS-cyclodextrin complex formulated into a transmucosal dosage form which is substantially free of cyclodextrin in excess of the minimum amount needed to maximize the amount of the S-CDS in the complex. In a particular aspect of the method, the composition administered comprises an essentially saturated S-CDS-cyclodextrin complex formulated into a transmucosal dosage form which is substantially free of cyclodextrin in excess of the minimum amount needed to maintain substantially all of the S-CDS in the complex, the amount of S-CDS in the complex being an amount effective to elicit a therapeutic response while maintaining acceptably low peripheral steroid levels.

The invention further provides a method for enhancing the bioavailability of a chemical delivery system for a steroidal sex hormone or an anti-inflammatory steroid (S-CDS) from a transmucosal dosage form in a mammal in need of treatment with said S-CDS, the method comprising: (a) determining the minimum amount of cyclodextrin required to complex with a selected amount of S-CDS and to maintain said selected amount of S-CDS in the complex; (b) combining an amount of S-CDS equal to or in excess of said selected amount with said minimum amount of cyclodextrin in an aqueous medium; (c) removing uncomplexed S-CDS, if any, from the complexation medium; (d) removing water from the resultant solution to afford the dry saturated S-CDS-cyclodextrin complex; (e) formulating said dry essentially saturated S-CDS-cyclodextrin complex into a transmucosal dosage form substantially free of cyclodextrin in excess of the minimum amount required to maximize the amount of S-CDS in the complex; and (f) administering the dosage form transmucosally to the mammal. In a particular aspect of this method, step (e) comprises formulating said dry essentially saturated S-CDS-cyclodextrin complex into a transmucosal dosage form substantially free of cyclodextrin in excess of the minimum amount required to maintain substantially all of the S-CDS in the complex, the amount of S-CDS in the complex being an amount effective to elicit a therapeutic response while maintaining acceptably low peripheral steroid levels.

The invention further provides for treatment of conditions responsive to administration of an S-CDS in mammals by administering thereto the composition of the invention. Use of an S-CDS in the preparation of the pharmaceutical compositions of the invention for administration to treat symptoms of S-CDS-responsive conditions and for enhancing the transmucosal bioavailability of an S-CDS is also provided.

### BRIEF DESCRIPTION OF THE DRAWINGS

A more complete appreciation of the invention and its many attendant advantages will be readily understood by reference to the following detailed description and the accompanying drawings, wherein the Figures are as follows.

FIG. 1 is a graphical representation of the results of phase solubility studies, where various cyclodextrin (CD) molar concentrations are plotted against various molar concentrations of estradiol-CDS, i.e. 17β-[(1-methyl-1,4-dihydro-3-pyridinyl)carbonyloxy]estra-1,3,5(10)-trien-3-ol (E₂-CDS), with ( ) representing hydroxypropyl-β-cyclodextrin.

FIG. 2 is a plot of lordosis quotient (percent responders) versus time in days for varying doses of E₂-CDS, at 0.003 mg/kg ( ), 0.01 mg/kg ( ), 0.03 mg/kg ( ), and of the control vehicle, hydroxypropyl-β-cyclodextrin (HPβCD) solution ( ), in ovariectomized female rats after daily intravenous (i.v.) injections for five days, with observations beginning on day 3 following the first injection.

FIG. 3 is a plot of lordosis quotient (percent responders) versus time in days for varying doses of estradiol benzoate, at 0.003 mg/kg (Δ), 0.01 mg/kg ( ) and 0.03 mg/kg ( ), and of the control vehicle, hydroxypropyl-β-cyclodextrin (HPβCD) solution (˜), in ovariectomized female rats after daily intravenous (i.v.) injections for five days, with observations beginning on day 3 following the first injection.

FIG. 4 is a group of three (3) plots of lordosis quotient (percent responders) versus time in days for the same doses as in FIGS. 2 and 3, but grouped so as to compare the same doses of E₂-CDS and estradiol benzoate.

FIG. 5 is a plot of LH levels in ng/mL plasma versus time in days for varying doses of E₂-CDS at 0.003 mg/kg ( ), 0.01 mg/kg ( ), 0.03 mg/kg ( ), and of the control ( ) in ovariectomized female rats after daily single i.v. tail injections for five days, with observations beginning on day 3 following the first injection.

FIG. 6 is a plot of LH levels in ng/mL plasma versus time in days for varying doses of estradiol benzoate at 0.003 mg/kg ( ), 0.01 mg/kg ( ), 0.03 mg/kg ( ), and of the control ( ) in ovariectomized female rats after daily single i.v. tail injections for five days, with observations beginning on day 3 following the first injection.

FIG. 7 is a bar graph illustrating the effect of varying doses of estradiol-CDS (E₂-CDS), at 0.03 mg/kg ( [symbol] ), 0.3 mg/kg ( [symbol] ), 3.0 mg/kg ( ), and of the control vehicle, hydroxypropyl-β-cyclodextrin (HPβCD) solution (˜), on the mounting performance (% responders) in intact male rats, and in castrated male rats at days 0, 3, 7, 14, 21, 28 and 35, after a single intravenous (i.v.) injection.

FIG. 8 is a bar graph illustrating the effect of varying doses E₂-CDS, at 0.03 mg/kg ( [symbol] ), 0.3 mg/kg ( [symbol] ) and 3.0 mg/kg ( ) and of the control vehicle, HPβCD (˜), on the intromission percentage (% responders) in intact male rats, and in castrated male rats at days 0, 3, 7, 14, 21, 28 and 35 after a single intravenous (i.v.) injection.

FIG. 9 is a bar graph and accompanying chart illustrating the effect of varying doses of E₂-CDS, at 0.03 mg/kg ( [symbol] ), 0.3 mg/kg ( [symbol] ), and 3.0 mg/kg ( ) and of the control vehicle HPβCD (˜), on the mounting frequency in intact male rats, and in castrated male rats at days 0, 3, 7, 14, 21, 28 and 35 after a single intravenous (i.v.) injection.

FIG. 10 is a bar graph and accompanying chart illustrating the effect of varying doses of E₂-CDS, at 0.03 mg/kg ( [symbol] ), 0.3 mg/kg ( [symbol] ) and 3 mg/kg ( ), and of the control vehicle HPβCD (˜), on the mounting latency, in minutes, in intact male rats, and in castrated male rats at days 0, 3, 7, 14, 21, 28 and 35 after a single intravenous (i.v.) injection.

FIG. 11 is a bar graph and accompanying chart illustrating the effect of varying doses of E₂-CDS, at 0.03 mg/kg ( [symbol] ), 0.3 mg/kg ( [symbol] ) and 3 mg/kg ( ), and of the control vehicle HPβCD (˜), on the intromission frequency in intact male rats, and in castrated male rats at days 0, 3, 7, 14, 21, 28 and 35 after a single intravenous (i.v.) injection.

FIG. 12 is a bar graph and accompanying chart illustrating the effect of varying doses of E₂-CDS, at 0.03 mg/kg ( [symbol] ), 0.3 mg/kg ( [symbol] ) and 3 mg/kg ( ) and of the control vehicle HPβCD (˜), on the intromission latency, in minutes, in intact male rats, and in castrated male rats at days 0, 3, 7, 14, 21, 28 and 35 after a single intravenous (i.v.) injection.

FIG. 13 is a plot of LH levels in ng/mL plasma versus time in days for varying doses of E₂-CDS at 0.03 mg/kg (x), 0.3 mg/kg ( ) and 3 mg/kg ( ) and of the control vehicle HPβCD ( ) in orchidectomized (castrated) male rats for a period of 35 days after a single intravenous (i.v.) injection.

FIG. 14 is a bar graph illustrating the effect of 0.03 mg/kg ( [symbol] ) E₂-CDS administered i.v. once, and 0.01 mg/kg ( [symbol] ) E₂-CDS administered i.v. once daily for 10 days, and the control vehicle, HPβCD (˜), on the mounting performance (% responders) in intact male rats, and in castrated male rats at days 0, 1, 3, 7, 14 and 21.

FIG. 15 is a bar graph illustrating the effect of 0.03 mg/kg ( [symbol] ) E₂-CDS administered i.v. once, and 0.01 mg/kg ( [symbol] ) E₂-CDS administered i.v. once daily for 10 days, and the control vehicle, HPβCD (˜), on the intromission performance (% responders) in intact male rats, and in castrated male rats at days 0, 1, 3, 7, 14 and 21.

FIG. 16 is a bar graph and accompanying chart illustrating the effect of 0.03 mg/kg ( [symbol] ) E₂-CDS administered i.v. once, and 0.01 mg/kg ( [symbol] ) E₂-CDS administered i.v. once daily for 10 days, and the control vehicle, HPβCD (˜), on the mounting frequency (number of mounts), in intact male rats, and in castrated male rats at days 0, 1, 3, 7, 14 and 21.

FIG. 17 is a bar graph and accompanying chart illustrating the effect of 0.03 mg/kg ( [symbol] ) E₂-CDS administered i.v. once, and 0.01 mg/kg ( [symbol] ) E₂-CDS administered i.v. once daily for 10 days, and the control vehicle, HPβCD(˜), on the mounting latency, in minutes, in intact male rats, and in castrated male rats at days 0, 1, 3, 7, 14 and 21.

FIG. 18 is a bar graph and accompanying chart illustrating the effect of 0.03 mg/kg ( [symbol] ) E₂-CDS administered i.v. once, and 0.01 mg/kg ( [symbol] ) E₂-CDS administered i.v. once daily for 10 days, and the control vehicle, HPβCD(˜), on the intromission latency, in minutes, in intact male rats, and in castrated male rats at days 0, 1, 3, 7, 14 and 21.

FIG. 19 is a bar graph and accompanying chart illustrating the effect of 0.03 mg/kg ( [symbol] ) E₂-CDS administered i.v. once, and 0.01 mg/kg ( [symbol] ) E₂-CDS administered i.v. once daily for 10 days, and the control vehicle, HPβCD (˜), on the intromission frequency (number of intromissions) in intact male rats, and in castrated male rats at days 0, 1, 3, 7, 14 and 21.

FIG. 20 is a plot of LH levels in ng/mL plasma versus time in days for a dose of 0.03 mg/kg (x) E₂-CDS administered i.v. once, a dose of 0.01 mg/kg ( ) E₂-CDS administered i.v. once daily for 10 days and of the control vehicle HPβCD( ) in orchidectomized (castrated) male rats for a period of 14 days.

FIG. 21 is a plot showing the effect of a single i.v. injection in rats of dexamethasone [DEX, (˜)] or 9-fluoro-11β,17-dihydroxy-16α-methyl-21 {[(1-methyl-1,4-dihydropyridin-3-yl)carbonyl]oxy}pregna-1,4-diene-3-one [DEX-CDS, ( )] on the per cent inhibition of stress-induced elevation of ACTH when subjected to a 5 minute stress test (upper portion) or a 15 minute stress test (lower portion).

FIG. 22 is a plot showing the per cent suppression of stress-induced elevation of corticosterone levels for the 15 minute stress test referred to in connection with FIG. 21.

### DETAILED DESCRIPTION OF THE INVENTION

Throughout the instant specification and claims, the following definitions and general statements are applicable.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

The term “complex” as used herein means an inclusion complex, in which a hydrophobic portion of the steroidal CDS molecule (typically a portion of the steroidal ring system) is inserted into the hydrophobic cavity of the cyclodextrin molecule. For example, in the case of E₂-CDS and HPβCD, it is believed that in the 1:1 complex, the aromatic A ring of the steroid is included. At higher HPβCD concentrations, a 1:2 complex of E₂-CDS:HPβCD forms and the second HPβCD molecule may interact with the dihydronicotinate group in the E₂-CDS molecule.

As used herein, whether in a transitional phrase or in the body of a claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a composition, the term “comprising” means that the composition includes at least the recited features or components, but may also include additional features or components.

The terms “consists essentially of” or “consisting essentially of” have a partially closed meaning, that is, they do not permit inclusion of steps or features or components which would substantially change the essential characteristics of a process or composition; for example, steps or features or components which would significantly interfere with the desired properties of the compositions described herein, i.e., the process or composition is limited to the specified steps or materials and those which do not materially affect the basic and novel characteristics of the invention. The basic and novel features herein are the provision of a saturated S-CDS-cyclodextrin complex in a transmucosal dosage form which is substantially free of cyclodextrin in excess of the minimum amount required to maximize the amount of S-CDS in the complex, the amount of S-CDS in the complex being an amount effective to elicit a therapeutic response while maintaining acceptably low peripheral steroid levels, and/or to provide improved bioavailability and/or lower interpatient and/or intrapatient variation following administration. In a particular embodiment of the invention, the basic and novel features herein are the provision of a saturated S-CDS-cyclodextrin complex in a transmucosal dosage form which is substantially free of cyclodextrin in excess of the minimum amount required to maintain substantially all of the S-CDS in the complex, the amount of S-CDS in the complex being an amount effective to elicit a therapeutic response while maintaining acceptably low peripheral steroid levels, and/or providing particularly enhanced bioavailability and/or low interpatient and/or low intrapatient variability following administration. In another embodiment, the basic and novel features herein are the provision of a buccal tablet, buccal wafer or buccal patch comprising an anhydrous formulation of a substantially saturated complex of the compound 17β-[1-methyl-1,4-dihydro-3-pyridinyl)carbonyloxy]estra-1,3,5(10)-trien-3-ol with a hydroxyalkyl, carboxyalkyl or carboxymethylethyl derivative of β- or γ-cyclodextrin comprising from about 0.01 to about 2.0 mg of said compound.

The terms “consists of” and “consists” are closed terminology and allow only for the inclusion of the recited steps or features or components.

As used herein, the singular forms “a,” “an” and “the” specifically also encompass the plural forms of the terms to which they refer, unless the content clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

The term “saturated” when used in conjunction with a complex of an S-CDS in cyclodextrin means that the complex is saturated with the S-CDS, that is, the complex contains the maximum amount of the S-CDS which can be complexed with a given amount of cyclodextrin under the conditions of complexation used. A phase solubility study can be used to provide this information, as described in more detail hereinafter. (Conditions for the complexation are also described in more detail below.) Alternatively, a saturated complex may be arrived at empirically by simply adding the S-CDS to an aqueous solution of the selected cyclodextrin until a precipitate (of uncomplexed S-CDS) forms; ultimately, the precipitate is removed and the solution lyophilized to provide the dry saturated complex.

The term “essentially”, as in “essentially saturated” means that from 80% to 100%, preferably from 90% to 100%, of the complex is in saturated form.

The expression “substantially”, as in “substantially free” or “substantially all”, means within 20% of the exact calculated amount. In the case of the expression “substantially free of cyclodextrin in excess of the minimum amount needed to maintain substantially all of the S-CDS in the complex,” the minimum amount of cyclodextrin needed to maintain the S-CDS in the complex can be obtained from phase solubility studies as explained in more detail below. The actual amount of cyclodextrin should be within 20% of that minimum, plus or minus, preferably within 10% of that minimum, plus or minus, even more preferably within 5% of that minimum, plus or minus, and should maintain at least 90% or more, preferably at least 95% or more, of the drug in the complex. On the other hand, when the expression “substantially free of cyclodextrin in excess of the minimum amount needed to maximize the amount of the S-CDS in the complex” is used, less than the aforenoted amount of cyclodextrin may be utilized and a larger amount of S-CDS may be present in the dosage form in uncomplexed form as a result. This may occur by using a less concentrated cyclodextrin solution for the complexation reaction and/or by conducting the complexation at the upper end of the temperature range suggested below. It is considered particularly advantageous, however, to use enough cyclodextrin to maintain substantially all of the S-CDS in the complex, and to thus minimize the amount of uncomplexed S-CDS in the dosage form.

The term “interpatient variability” refers to variation among patients to which a drug is administered. The term “intrapatient variability” refers to variation experienced by a single patient when dosed at different times.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001).

The term “S-CDS” as used herein means a drug which is a brain-specific chemical delivery system for a steroidal sex hormone or an anti-inflammatory steroid. This is a narrower definition than “CDS” as used in the art, which describes brain-specific chemicals delivery systems for many different kinds of centrally acting drugs; however, it is a useful shorthand way to refer to the drugs to which the present invention relates. More particularly, the term “S-CDS” as used in describing the present invention represents a compound of the formula:
DDHC]ₙ (I)
or a non-toxic pharmaceutically acceptable salt thereof, wherein:
(a) D is the residue of a steroidal female sex hormone having one or two reactive hydroxyl functional groups, one such hydroxyl group being a 17β-hydroxy substituent, said residue having a hydrogen atom absent from at least one of said reactive hydroxyl functional groups; n is a positive integer equal to the number of said functional groups from which a hydrogen atom is absent; and [DHC] is a radical of the formula 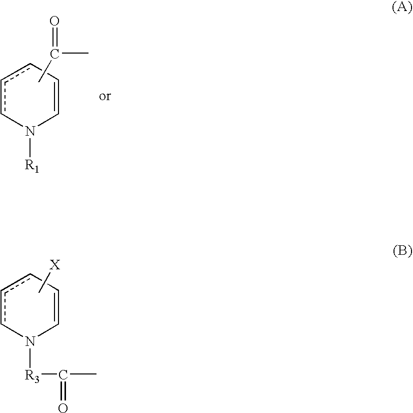
   wherein the dotted line indicates the presence of a double bond in either the 4- or 5-position of the dihydropyridine ring; R₁ is C₁-C₇ alkyl or C₇-C₁₀ aralkyl; R₃ is C₁-C₃ alkylene; X is —CONR′R″ wherein each of R′ and R″, which are the same or different, is H or C₁-C₇ alkyl, or X is —COOR′″ wherein R′″ is C₁-C₇ alkyl or C₇-C₁₀ aralkyl; the carbonyl grouping in (A) is attached at the 2-, 3- or 4-position of the dihydropyridine ring; and the X grouping in (B) is attached at the 2-, 3- or 4-position of the dihydropyridine ring;
(b) D is the residue of an anti-inflammatory steroid having at least one reactive hydroxyl functional group, one such hydroxyl group being a 21-hydroxy substituent, said residue being characterized by the absence of a hydrogen atom from at least one of said reactive hydroxyl functional groups; and n and [DHC] are defined as above; or
(c) D is the residue of a steroidal androgen having a reactive 17β-hydroxyl functional group, said residue having a hydrogen atom absent from the 17β-hydroxyl functional group; n is one and [DHC] is defined as above.

In the foregoing formulas, n is generally 1 or 2; in a number of specific embodiments, n is 1. When R₁, R′, R″ or R′″ is C₁-C₇ alkyl, it can be methyl, ethyl, propyl, butyl, hexyl or heptyl or one of the branched-chain isomers thereof. When R₁ or R′″ is C₇-C₁₀ aralkyl, it is —(C₁-C₃ alkylene)phenyl, typically benzyl. In a number of specific embodiments, [DHC] has formula (A) in which R₁ is methyl. When [DHC] has formula (B), R₃ is typically —CH₂— and X is typically —CONH₂ or —COOR′″ wherein R′″ is typically methyl or ethyl.

When D is the residue of a steroidal female sex hormone as defined in (a) above, it is the residue of a steroidal estrogen or a steroidal progestin having the structural requirements in the definition above. Such estrogens include, for example, estradiol, ethinyl estradiol, estrone, estradiol 3-methyl ether, estradiol benzoate and mestranol; such progestins include, for example, norethindrone, ethisterone, norgestrel and norethynodrel. Exemplary compounds of formula (I) wherein D is as defined in (a) above include the following:

| | | Abbreviated |
|---|---|---|
| Structure | Chemical Name | Name |
| 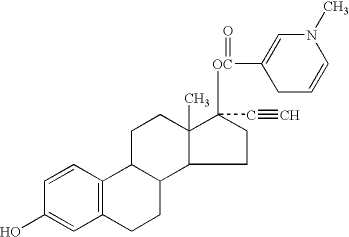 | 3-hydroxy-17β-{[1- methyl-1,4- dihydropyridin-3- yl)carbonyl]oxy}-19- nor-17α-pregna- 1,3,5(10)-trien-20-yne | ethinyl estradiol-CDS |
| 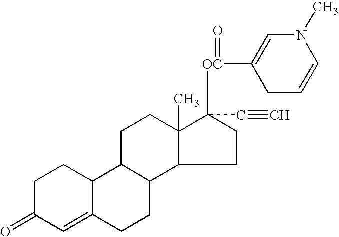 | 17α{[(1-methyl-1,4- dihydropyridin-3- yl)carbonyl]oxy}-19- norpregn-4-en-20-yn-3- one | norethindrone- CDS |
| 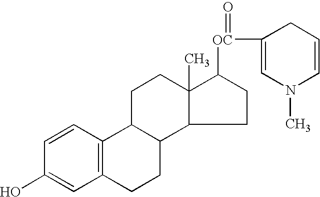 | 17β-[(1-methyl-1,4- dihydropyridin-3- yl)carbonyloxy]estra- 1,3,5(10)-trien-3-ol | estradiol-CDS or E₂-CDS |
| 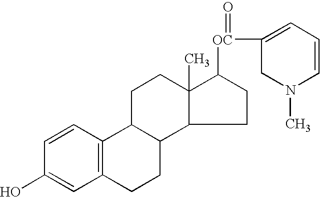 | 17β-[(1-methyl-1,2- dihydropyridin-3- yl)carbonyloxy]estra- 1,3,5(10)-trien-3-ol | estradiol-CDS_{1,2}or E₂-CDS_{1,2} |
| 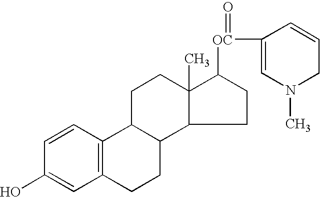 | 17β-[(1-methyl-1,6- dihydropyridin-3- yl)carbonyloxy]estra- 1,3,5(10)-trien-3-ol | estradiol-CDS_{1,6}or E₂-CDS_{1.6} |
| 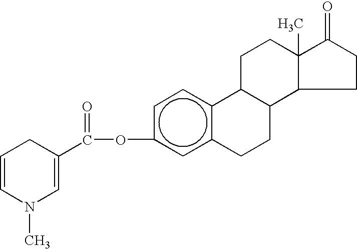 | 3-[(1-methyl-1,4- dihydro-3- pyridinyl)carbonyloxy]estra-1,3,5(10)-trien- 17-one | estrone-CDS |
| 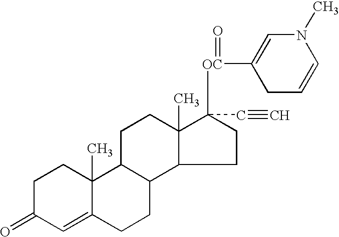 | 17β{[(1-methyl-1,4- dihydropyridin-3- yl)carbonyl]oxy}preg- 4-en-20-yn-3 -one | ethisterone-CDS |
| 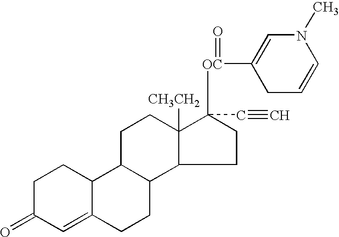 | 13-ethyl-17β{[(1- methyl-1,4- dihydropyridin-3- yl)carbonyl]oxy}- 18,19-dinorpregn-4- ene-20-yn-3-one | norgestrel-CDS |
| 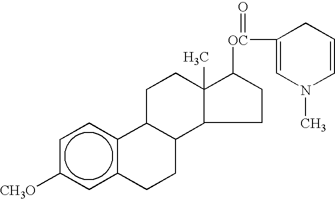 | 17β-[(1-methyl-1,4- dihydro-3- pyridinyl)carbonyloxy]estra-1,3,5(10)-trien-3- ol 3-methyl ether | estradiol 3- methyl ether- CDS |
| 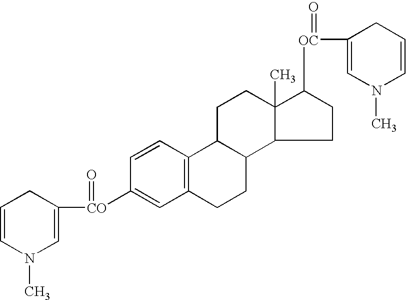 | 3,17β-bis{[(1-methyl- 1,4-dihydropyridin-3- yl)carbonyl]oxy}estra- 1,3,5(10)-triene | estradiol bis-CDS |
| 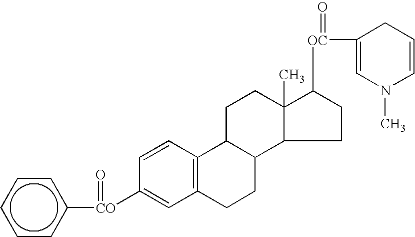 | 3-(phenylcarbonyloxy)- 17β-{[(1-methyl-1,4- dihydropyridin-3-yl)- carbonyl]oxy}estra- 1,3,5(10)-triene | estradiol benzoate-CDS |
| 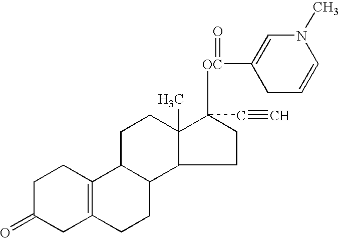 | 17β-{[(1-methyl-1,4- dihydropyridin-3- yl)carbonyl]oxy}-19- norpregn-5(10)-en-20- yn-3-one | norethynodrel- CDS |
| 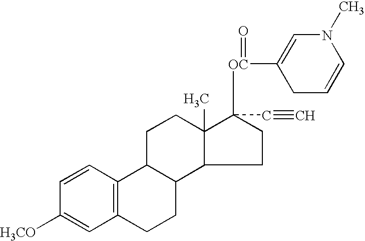 | 3-methoxy-17α-{[(1- methyl-1,4- dihydropyridin-3- yl)carbonyl]oxy}-19 nor-17α-pregna- 1,3,5(10)-trien-20-yne | mestranol-CDS |

The compounds in which D is defined as in (a) above can be prepared by methods described in the art or methods analogous thereto; see, for example, Bodor U.S. Pat. Nos. 4,900,837 and 5,017,566 and references cited therein. For example, when [DHC] is (A) above, the hydroxy group in the drug reacts with nicotinoyl chloride, the nicotinate which forms is subsequently quaternized, e.g. with methyl iodide, and the quaternary salt is thereafter reduced, e.g. with sodium dithionate. The 1,4-dihydropyridine derivative is formed in this manner, with small amounts of the 1,6-dihydropyridine and 1,2-dihydropyridine compounds also being formed in the reaction mixture. The 1,6- and 1,2-dihydropyridine derivatives can be formed predominantly using sodium borohydride reduction. In any event, the 1,4-, 1,6 and 1,2-dihydropyridine derivatives are all oxidized to the same quaternary form in vivo, that is, the form locked-in the brain which ultimately releases the active drug, for example, estradiol. When [DHC] is (B) above, the hydroxy group in the drug can be reacted with bromoacetyl chloride to convert the —OH to a —OCOCH₂Br group; that compound can then be reacted with a nicotinic acid ester or amide to form the corresponding quaternary salt, which is then reduced to give the desired compound.

A preferred compound of formula (I) wherein D is as defined in (a) above is the compound identified as E₂-CDS above.

When D is the residue of an anti-inflammatory steroid as defined in (b) above, it is, for example, the residue of dexamethasone, hydrocortisone, betamethasone, cortisone, flumethasone, fluprednisolone, meprednisone, methylprednisolone, prednisolone, prednisone, triamcinolone, cortodoxone, fludrocortisone, fluandrenolide, or paramethasone. Illustrative compounds of formula (I) wherein D is as defined in (b) include the following:

| | | Abbreviated |
|---|---|---|
| Structure | Chemical Name | Name |
| 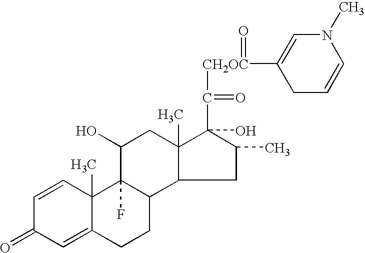 | 9-fluoro-11β,17- dihydroxy-16α-methyl- 21-{[(1-methyl-1,4- dihydropyridin-3- yl)carbonyl]oxy}pregna- 1,4-diene-3,20-dione | dexamethasone- CDS or DEX- CDS |
| 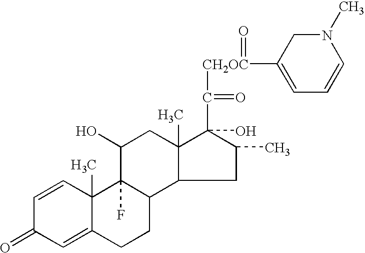 | 9-fluoro-11β,17- dihydroxy-16α-methyl- 21-{[(1-methyl-1,2- dihydropyridin-3- yl)carbonyl]oxy}pregna- 1,4-diene-3,20-dione | dexamethasone- CDS_{1,2} or DEX- CDS_{1,2} |
| 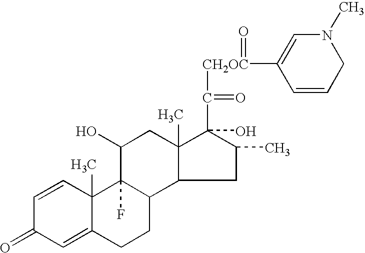 | 9-fluoro-11β,17- dihydroxy-16α- methyl-21-{[(1- methyl-1,6- dihydropyridin-3- yl)carbonyl]oxy}pregna- 1,4-diene-3,20-dione | dexamethasone- CDS_{1,6} or DEX- CDS_{1.6} |
| 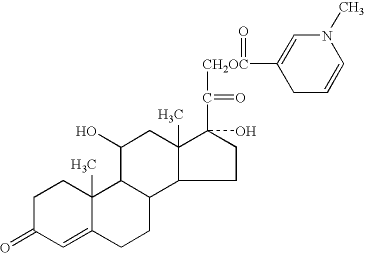 | 11β,17-dihydroxy-21- {[(1-methyl-1,4- dihydropyridin-3- yl)carbonyl]oxy}pregn- 4-ene-3,20-dione | hydrocortisone- CDS |

The compounds in which D is defined as in (b) above can be prepared by methods described in the art or methods analogous thereto; see, for example, Bodor U.S. Pat. Nos. 4,880,921 and 5,017,566 and references cited therein; see also the procedures outlined above for the steroids in which D is a residue as defined in (a) above.

A preferred compound of formula (I) wherein D is a defined in (b) above is the compound identified as dexamethasone-CDS or DEX-CDS above.

When D is the residue of a steroidal androgen as defined in (c) above, it is, for example, the residue testosterone or methyltestosterone.

The compounds in which D is defined as in (c) above can be prepared by methods described in the art or methods analogous thereto; see, for example, Bodor U.S. Pat. Nos. 4,479,932; 4,900,837 and 5,017,566 and references cited therein; see also the procedures outlined above for the steroids in which D is a residue as defined in (a) above. Illustrations of the compounds of formula (I) in which D is defined as in (c) above are the following:

| | | Abbreviated |
|---|---|---|
| Structure | Chemical Name | Name |
| 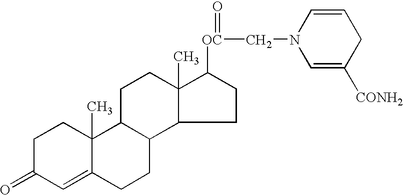 | 17β-{[(3″-carbamoyl-1′,4′- dihydropyridinyl)acetyl]oxy}-androst-4-en-3-one | testoterone- CDS₂ or T-CDS₂ |
| 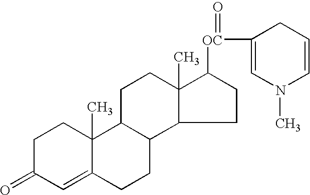 | 17β-[(1,4-dihydro-1- methyl-3- pyridinylcarbonyl)oxy]androst-4-en-3-one | testerone- CDS₁ or T-CDS₁ |

A preferred compound of formula (I) wherein D is as defined in (c) above is the compound identified as T-CDS₁ above.

Reference is made herein in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In this description, numerous specific details are set forth in order to provide a thorough understanding of the present invention. The present invention may be practiced without some or all of these specific details. In other instances, well-known process operations have not been described in detail, in order not to unnecessarily obscure the present invention.

There is provided by the present invention compositions, as well as methods of making and of using pharmaceutical compositions, useful to achieve desirable pharmacokinetic properties. Such compositions stem from the belief that solutions of cyclodextrin and an S-CDS in which the S-CDS is in its highest thermodynamic state, when presented to the mucosa through which they are absorbed (nasal, rectal, sublingual, vaginal or, especially, buccal) are associated with improved S-CDS absorption, as reflected by higher bioavailability and/or lower interpatient and/or intrapatient variation, enabling lowering of the dosage administered so as to maintain acceptably low peripheral steroid levels.

It is postulated, without wishing to so limit the invention, that upon dissolution (e.g., by contact with a fluid, such as a bodily fluid), dry compositions of an essentially saturated S-CDS-cyclodextrin complex not containing excess cyclodextrin form a locally essentially saturated S-CDS solution in which the S-CDS is in the state of highest thermodynamic activity (HTA), thus favoring absorption. The free S-CDS formed from dissociation of the complex in an essentially saturated aqueous solution seeks a more stable activity level, and if excess cyclodextrin were present, the S-CDS would seek greater stability by re-complexing with the cyclodextrin. By controlling the amount of cyclodextrin so that the dosage form is substantially free of cyclodextrin in excess of the amount needed to keep the S-CDS in the complex, it will not be easy for the S-CDS in the locally saturated solution to recombine with cyclodextrin. Therefore, this S-CDS will seek a state of lower thermodynamic activity/greater stability by being absorbed through the nasal, buccal, sublingual, vaginal or rectal mucosa. This approach is believed inter alia to increase bioavailability, likely by avoiding or minimizing the inhibition of S-CDS absorption which would result from the presence of excess cyclodextrin. In the presence of a large amount of excess cyclodextrin, the S-CDS in solution would be expected to recombine with cyclodextrin. This will not achieve optimum bioavailability, because it is essential that the S-CDS move out of the complex in which it is encapsulated if the drug is to accomplish its therapeutic function.

In view of the foregoing, it is apparent that to produce optimal pharmaceutical compositions, in a solid transmucosal dosage form, these dosage forms should be formulated to release a localized essentially saturated S-CDS solution, upon contact of the solid dosage forms with body fluid at the mucosa, in which the S-CDS is in its HTA state. To provide such a localized essentially saturated solution in vivo, it is important to first identify the optimal ratio of S-CDS to cyclodextrin, which ratio is referred to herein as the HTA ratio, to be used in the solid dosage form. In the case of a buccal dosage form, a highly concentrated solution made by dissolving the essentially saturated complex in a minimal amount of water and placing this solution in the buccal cavity can accomplish the same effect.

The HTA ratio is empirically determined and is identified as the ratio of the S-CDS to a specific cyclodextrin which corresponds to the maximum amount of the S-CDS that can be complexed with a given amount of cyclodextrin. The HTA ratio may be determined using an empirical method such as a phase solubility study to determine the saturation concentration of the S-CDS that can be solubilized with different concentrations of cyclodextrin solutions. Hence, the method identifies the concentrations at which a saturated S-CDS-cyclodextrin complex is formed. It is noted that the molar ratio represented by a point on the phase solubility graph shows how many moles of cyclodextrin are the minimum needed to maintain the drug in the complex, under given conditions; this may then be converted to a weight ratio. For example, if a phase solubility diagram shows that a given number of moles of a given cyclodextrin are needed to maintain substantially all of the S-CDS in a saturated complex, then multiplying the number of moles of the S-CDS by its molecular weight and multiplying the number of moles of the cyclodextrin by its molecular weight, one can arrive at the ratio of the products as an appropriate optimized weight ratio. A phase solubility study also provides information about the nature of the S-CDS-cyclodextrin complex formed, for example whether the complex is a 1:1 complex (1 molecule of drug complexed with 1 molecule of cyclodextrin) or a 1:2 complex (1 molecule of drug complexed with 2 molecules of cyclodextrin).

In accordance with the present invention, one can start using either cyclodextrin or the S-CDS as the fixed variable to which an excess of the other is added to identify various HTA data points (indicating saturated S-CDS-cyclodextrin complexes) and draw the resultant HTA line. Typically, the S-CDS is added to an aqueous solution having a known concentration of cyclodextrin under conditions empirically found to promote complex formation. A concentrated solution, for example, of approximately 25% for hydroxypropyl-γ-cyclodextrin and approximately 33 to 40% for hydroxypropyl-β-cyclodextrin, is in one embodiment particularly advantageous. Generally, the complexation is conducted at room temperature, although slight heating (up to about 50° C. or even up to 60° C.) may be employed. Excess S-CDS, if any, is then removed and the S-CDS concentration in the complex is subsequently measured. The concentration measured represents the S-CDS saturation concentration for the given cyclodextrin concentration. This process is repeated for a different known concentration of cyclodextrin until several data points are obtained. Each data point represents the saturated concentration of the S-CDS dissolved in a known concentration of cyclodextrin. The data points are then plotted to show the saturated concentration of S-CDS against the various cyclodextrin concentrations used. The graph is a phase solubility diagram which can be used to determine the saturation amount of the S-CDS for any specific concentration of cyclodextrin used to form a saturated S-CDS-cyclodextrin complex under a given set of complexation conditions.

One of skill in the art will appreciate that concentrations at which saturated S-CDS-cyclodextrin complexes are formed (and thus HTA ratios as well) may be identified by a variety of alternative methodologies. Accordingly, any method known in the field suitable to identify these concentrations is within the scope of the invention.

The cyclodextrins within the scope of this invention include the natural cyclodextrins α-, β, and γ-cyclodextrin, and derivatives thereof, in particular, derivatives wherein one or more of the hydroxy groups are substituted, for example, by alkyl, hydroxyalkyl, carboxyalkyl, alkylcarbonyl, carboxyalkoxyalkyl, alkylcarbonyloxyalkyl, alkoxycarbonylalkyl or hydroxy-(mono or polyalkoxy)alkyl groups; and wherein each alkyl or alkylene moiety preferably contains up to six carbons. Substituted cyclodextrins can generally be obtained in varying degrees of substitution, for example, from 1 to 14, preferably from 4 to 7; the degree of substitution is the approximate average number of substituent groups on the cyclodextrin molecule, for example, the approximate number of hydroxypropyl groups in the case of the hydroxypropyl-β-cyclodextrin molecule, and all such variations are within the ambit of this invention. Substituted cyclodextrins which can be used in the invention include polyethers, for example, as described in U.S. Pat. No. 3,459,731. Further examples of substituted cyclodextrins include ethers wherein the hydrogen of one or more cyclodextrin hydroxy groups is replaced by C₁₋₆-alkyl, hydroxy-C₁₋₆ alkyl, carboxy-C₁₋₆ alkyl or C₁₋₆ alkyloxycarbonyl-C₁₋₆ alkyl groups or mixed ethers thereof. In particular, such substituted cyclodextrins are ethers wherein the hydrogen of one or more cyclodextrin hydroxy groups is replaced by C₁₋₃ alkyl, hydroxy-C₂₋₄ alkyl or carboxy-C₁₋₂ alkyl or more particularly by methyl, ethyl, hydroxyethyl, hydroxypropyl, hydroxybutyl, carboxymethyl or carboxyethyl. As an example of a mixed ether, one can cite O-carboxymethyl-O-ethyl-β-cyclodextrin, also referred to as carboxymethylethyl-β-cyclodextrin and similar mixed ethers such as carboxymethylethyl-γ-cyclodextrin. The term “C₁₋₆ alkyl” is meant to include straight and branched saturated hydrocarbon radicals, having from 1 to 6 carbon atoms such as methyl, ethyl, 1-methylethyl, 1,1-dimethylethyl, propyl, 2-methylpropyl, butyl, pentyl, hexyl and the like. Other cyclodextrins contemplated for use herein include glucosyl-β-cyclodextrin and maltosyl-β-cyclodextrin. Of particular utility in the present invention are the β-cyclodextrin ethers such as dimethyl-β-cyclodextrin as described in Cyclodextrins of the Future, Vol. 9, No. 8, p. 577-578 by M. Nogradi (1984), randomly methylated β-cyclodextrin and polyethers such as hydroxypropyl-β-cyclodextrin, hydroxyethyl-β-cyclodextrin, hydroxypropyl-γ-cyclodextrin, and hydroxyethyl-γ-cyclodextrin, as well as sulfobutyl ethers, especially β-cyclodextrin sulfobutyl ether. In addition to simple cyclodextrins, branched cyclodextrins and cyclodextrin polymers may also be used. Other cyclodextrins are described, for example, in Chemical and Pharmaceutical Bulletin 28: 1552-1558 (1980); Yakugyo Jiho No. 6452 (28 Mar. 1983); Angew. Chem. Int. Ed. Engl. 19: 344-362 (1980); U.S. Pat. Nos. 3,459,731 and 4,535,152; European Patent Publication Nos. EP 0 149 197A and EP 0 197 571A; PCT International Patent Publication No. WO90/12035; and UK Patent Publication GB 2,189,245. Other references describing cyclodextrins for use in the compositions according to the present invention, and which provide a guide for the preparation, purification and analysis of cyclodextrins include the following: Cyclodextrin Technology by Jozsef Szejtli, Kluwer Academic Publishers (1988) in the chapter Cyclodextrins in Pharmaceuticals; Cyclodextrin Chemistry by M. L. Bender et al., Springer-Verlag, Berlin (1978); Advances in Carbohydrate Chemistry, Vol. 12, Ed. by M. L. Wolfrom, Academic Press, New York in the chapter “The Schardinger Dextrins” by Dexter French, pp. 189-260; Cyclodextrins and their Inclusion Complexes by J. Szejtli, Akademiai Kiado, Budapest, Hungary (1982); I. Tabushi, Acc. Chem. Research, 1982, 15, pp. 66-72; W. Sanger, Angewandte Chemie, 92, pp. 343-361 (1981); A. P. Croft et al., Tetrahedron, 39, pp. 1417-1474 (1983); Irie et al., Pharmaceutical Research, 5, pp. 713-716 (1988); Pitha et al., Int. J. Pharm. 29, 73 (1986); U.S. Pat. Nos. 4,659,696 and 4,383,992; German Patent Nos. DE 3,118,218 and DE 3,317,064; and European Patent No. EP 0 094 157A. Patents describing hydroxyalkylated derivative of β- and γ-cyclodextrin include Pitha U.S. Pat. Nos. 4,596,795 and 4,727,064 and Müller U.S. Pat. Nos. 4,764,604 and 4,870,060 and Müller et al. U.S. Pat. No. 6,407,079.

Cyclodextrins of particular interest for complexation with an S-CDS (e.g. E₂-CDS, DEX-CDS or testosterone-CDS₁) include: hydroxyalkyl, e.g. hydroxyethyl or hydroxypropyl, derivatives of β- and γ-cyclodextrin; carboxyalkyl, e.g. carboxymethyl or carboxyethyl, derivatives of β- or γ-cyclodextrin; β-cyclodextrin sulfobutyl ether; carboxymethylethyl-β- or γ-cyclodextrin; dimethyl-β-cyclodextrin; and randomly methylated β-cyclodextrin. 2-Hydroxypropyl-β-cyclodextrin (HPβCD), 2-hydroxypropyl-γ-cyclodextrin (HPγCD), randomly methylated β-cyclodextrin, dimethyl-β-cyclodextrin, β-cyclodextrin sulfobutyl ether, carboxymethyl-β-cyclodextrin (CMβCD), carboxymethyl-γ-cyclodextrin (CMγCD) and carboxymethylethyl-β-cyclodextrin are of special interest, especially hydroxypropyl-β-cyclodextrin, hydroxypropyl-γ-cyclodextrin, carboxymethyl-β-cyclodextrin and carboxymethyl-γ-cyclodextrin.

Compositions of an essentially saturated S-CDS-cyclodextrin complex for use in the present invention can be prepared under conditions favoring complex formation in a liquid environment as described and as exemplified herein. The resultant liquid preparations can be subsequently converted to a dry form suitable for administration as a solid transmucosal dosage form.

One of skill will appreciate that a variety of approaches are available in the field to prepare compositions as described herein. One available method exemplified herein includes the steps of adding the S-CDS to an aqueous cyclodextrin solution, maintaining the complexation medium at room temperature or below, preferably with stirring, for a sufficient time to achieve equilibration (e.g. for from about 4 to about 24 hours), evaporating to dryness, reconstituting the residue, separating un-complexed S-CDS, if any, e.g. by filtering or centrifugation, and lyophilizing or freeze-drying the essentially saturated solution to form a solid essentially saturated S-CDS-cyclodextrin complex.

Freeze-drying, also known as lyophilization, consists of three basic stages: first a freezing stage, then a primary drying stage and finally a secondary drying phase. Lyophilization can be optimized by following the principles described by Xiaolin (Charlie) Tang and Michael J. Pikal in Pharmaceutical Research, Vol. 21, No. 2, February 2004, 191-200, incorporated by reference herein in its entirety and relied upon.

Pharmaceutical compositions according to the invention may optionally include one or more excipients or other pharmaceutically inert components. One of the advantages of the invention, however, is that S-CDS drug forms as described herein can be prepared with the minimal amount of excipients necessary for shaping and producing the particular form, such as a tablet or patch. Excipients may be chosen from those that do not interfere with the S-CDS, with cyclodextrin or with complex formation.

Dosage forms are optionally formulated in a pharmaceutically acceptable vehicle with any of the well-known pharmaceutically acceptable carriers, diluents, binders, lubricants, disintegrants, scavengers, flavoring agents, coloring agents, and excipients (see Handbook of Pharmaceutical Excipients, Marcel Dekker Inc., New York and Basel (1998); Lachman et al. Eds., The Theory and Practice of Industrial Pharmacy, 3^{rd} Ed., (1986); Lieberman et al., Eds. Pharmaceutical Dosage Forms, Marcel Dekker Inc., New York and Basel (1989); and The Handbook of Pharmaceutical Excipients, 3^{rd} Ed., American Pharmaceutical Association and Pharmaceutical Press, 2000); see also Remington's Pharmaceutical Sciences, 18th Ed., Gennaro, Mack Publishing Co., Easton, Pa. (1990) and Remington: The Science and Practice of Pharmacy, Lippincott, Williams & Wilkins, (1995)). A simple solid transmucosal dosage form consists of the essentially saturated S-CDS-cyclodextrin complex compressed with a small amount (e.g. about 1% by weight) of a suitable binder or lubricant such as magnesium stearate. Sorbitol may be added to the complex as well as magnesium stearate to aid in fast dissolution and to give good mouth feel.

In particular embodiments, the essentially saturated S-CDS-cyclodextrin complex is used for the transmucosal, especially buccal, administration of the S-CDS. The term “buccal” refers to delivery of a drug by passage of the drug through the buccal mucosa into the blood stream.

As used herein, “mucosa” means the epithelial membranes lining the nasal, oral, vaginal and rectal cavities. As used herein, mucosal and transmucosal are used interchangeably. Transmucosal delivery methods and forms are well-known in the art. These include buccal and sublingual tablets, lozenges, adhesive patches, gels, solutions or sprays (powder, liquid or aerosol), and suppositories or foams (for rectal or vaginal administration). When the transmucosal form is a liquid, it can be obtained by dissolving the essentially saturated complex in a minimum amount of water, for example 500 mg of the essentially saturated complex with HPβCD in 0.5 mL water (50% w/w solution), or 500 mg of the essentially saturated γCD complex in 1.0 mL of water. A few drops of such a solution can be inserted into the buccal cavity and retained there for about 2 minutes to allow for absorption through the buccal mucosa. Nevertheless, solid transmucosal dosage forms are generally preferred over liquid forms.

In certain instances, mucosal absorption may be further facilitated by the addition of various excipients and additives to increase solubility or to enhance penetration, such as by the modification of the microenvironment, or by the addition of mucoadhesive excipients to improve contact between the delivery system and the mucosal tissue.

Buccal drug delivery can be effected by placing the buccal dosage unit between the lower gum and the oral mucosa opposite thereto of the individual undergoing drug therapy. Excipients or vehicles suitable for buccal drug administration can be used, and include any such materials known in the art, e.g., any liquid, gel, solvent, liquid diluent, solubilizer, or the like, which is nontoxic and does not interact with other components of the composition in a deleterious manner. A solid dosage unit is fabricated so as to dissolve gradually over a predetermined time period, to produce a substantially saturated drug solution in the saliva of the buccal cavity, allowing absorption of the S-CDS (e.g. E₂-CDS, DEX-CDS or T-CDS₁) through the mucosa, wherein drug delivery is provided essentially throughout the time period. The buccal dosage unit may further comprise a lubricant to facilitate manufacture, e.g., magnesium stearate or the like. Additional components that may be included in the buccal dosage unit include but are not limited to flavorings, permeation enhancers, diluents, binders, and the like. The remainder of the buccal dosage unit may comprise a bioerodible polymeric carrier, and any excipients that may be desired, e.g., binders, disintegrants, lubricants, diluents, flavorings, colorings, and the like, and/or additional active agents.

The buccal carrier can comprise a polymer having sufficient tack to ensure that the dosage unit adheres to the buccal mucosa for the necessary time period, i.e., the time period during which the S-CDS is to be delivered to the buccal mucosa. Additionally, the polymeric carrier is gradually “bioerodible”, i.e., the polymer hydrolyzes at a predetermined rate upon contact with moisture. Any polymeric carriers can be used that are pharmaceutically acceptable, provide both a suitable degree of adhesion and the desired drug release profile, and are compatible with the S-CDS to be administered and any other components that may be present in the buccal dosage unit. Generally, the polymeric carriers comprise hydrophilic (water-soluble and water-swellable) polymers that adhere to the wet surface of the buccal mucosa. Examples of polymeric carriers useful herein include acrylic acid polymers and copolymers, e.g., those known as “carbomers” for example, Carbopol®. Other suitable polymers include, but are not limited to, hydrolyzed polyvinyl alcohol, polyethylene oxides (e.g., Sentry Polyox®), polyacrylates (e.g., Gantrez®), vinyl polymers and copolymers, polyvinylpyrrolidone, dextran, guar gum, pectins, starches, and cellulosic polymers such as hydroxypropyl methylcellulose (e.g., Methocel®), hydroxypropyl cellulose (e.g., Klucel®), hydroxypropyl cellulose ethers, hydroxyethyl cellulose, sodium carboxymethyl cellulose, methyl cellulose, ethyl cellulose, cellulose acetate phthalate, cellulose acetate butyrate, and the like. The dosage unit need contain only the S-CDS-cyclodextrin complex. However, it is generally desirable to include one or more of the aforenoted carriers and/or one or more additional components. For example, a lubricant may be included to facilitate the process of manufacturing the dosage units; lubricants may also optimize erosion rate and drug flux. If a lubricant is present, it will represent on the order of 0.01 wt. % to about 2 wt. %, preferably about 0.01 wt. % to 1.0 wt. %, of the dosage unit. Suitable lubricants include, but are not limited to, magnesium stearate, calcium stearate, stearic acid, sodium stearylfumarate, talc, hydrogenated vegetable oils and polyethylene glycol. In any event, the S-CDS (for example, E₂-CDS, DEX-CDS or T-CDS₁) will be incorporated into the buccal dosage form as a complex, preferably an essentially saturated complex, for example, with a hydroxyalkyl or carboxyalkyl or carboxymethylethyl derivative of β- or γ-cyclodextrin, with randomly methylated or γ-cyclodextrin, or with sulfobutyl β- or γ-cyclodextrin.

The essentially saturated S-CDS-cyclodextrin complex may also be administered in accord with this invention in the form of suppositories or foams for vaginal or rectal administration. These compositions can be prepared by well-known methods, for example, in the case of suppositories, by mixing the saturated complex with a suitable non-irritating excipient or binder which is solid at ordinary temperatures but liquid at the vaginal or rectal temperature and will, therefore, melt in the vagina or rectum to release the drug. Such materials include cocoa butter and polyethylene glycols. Traditional binders and carriers include, for example, polyalkylene glycols or triglycerides [e.g., PEG 1000 (96%) and PEG 4000 (4%)]. Such suppositories may be formed from mixtures containing active ingredients in the range of from about 0.5 wt/wt % to about 10 wt/wt %; preferably from about 1 wt/wt % to about 2 wt/wt %.

For intranasal use, a powder spray, suspension gel or ointment may be utilized, preferably a powder form of the essentially saturated complex.

Moreover, for use in humans, a buccal dosage form, especially a buccal tablet or wafer or disk, advantageously having a disintegration time of about 15-30 minutes, or a buccal patch (in which the drug is released only from the side which adheres to the buccal mucosa while the other side is nonpermeable), has particular advantages as it can be readily self-administered yet provides better bioavailability than oral dosage forms because the S-CDS passes directly into the bloodstream from the buccal mucosa. (The cyclodextrin derivative is not absorbed, of course.) The carrier moiety, for example the dihydrotrigonellinate moiety of E₂-CDS, shows instability in gastrointestinal fluid leading to multiple decomposition products starting with water addition and/or oxidation; buccal delivery also avoids hepatic first pass metabolism of the drug. The formulations for buccal administration are preferably anhydrous for reasons of storage stability.

In particularly advantageous embodiments of the invention, buccal administration may make use of the inventions of Nagai et al. described in U.S. Pat. Nos. 4,226,848 and 4,250,163, both of which are incorporated by reference herein in their entireties and relied upon. Thus, a buccal mucosa-adhesive tablet may be formulated for use herein comprising: (a) a water-swellable and mucosa-adhesive polymeric matrix comprising about 50% to about 95% by weight of a cellulose ether and about 50% to about 95% by weight of a homo- or copolymer of acrylic acid or a pharmaceutically acceptable salt thereof, and (b) dispersed therein, an appropriate quantity of the S-CDS (for example, E₂-CDS), as an essentially saturated complex with the selected cyclodextrin, for example, hydroxypropyl-β-cyclodextrin, hydroxypropyl-γ-cyclodextrin, carboxymethyl-β-cyclodextrin, carboxymethyl-γ-cyclodextrin, carboxymethylethyl-β-cyclodextrin, sulfobutyl β- or γ-cyclodextrin, or randomly methylated β- or γ-cyclodextrin. Ideally, for storage stability, the tablet is anhydrous.

The term “therapeutically effective amount” or “effective amount” or “an amount effective to elicit a therapeutic response” is used to denote treatments at dosages effective to achieve the therapeutic result sought. The therapeutic result sought of course depends upon the identity of the particular steroid which the S-CDS in the complex is intended to deliver, especially whether the steroid is an estrogen, a progestin, an androgen or an anti-inflammatory agent.

When the S-CDS in the complex is intended for delivery of an estrogen, for example, when the S-CDS is E₂-CDS, the effective amount, that is, the therapeutically effective amount, will be an amount of the S-CDS sufficient to produce a beneficial CNS-related estrogenic effect while maintaining acceptably low peripheral estrogen levels.

When the S-CDS in the complex is intended for delivery of a progestin, the effective amount, that is, the therapeutically effective amount, will be an amount of the S-CDS sufficient to produce a beneficial CNS-related progestational effect while maintaining acceptably low peripheral progestin levels.

When the S-CDS in the complex is intended for delivery of an anti-inflammatory steroid, for example, when the S-CDS is dexamethasone-CDS, the effective amount, that is, the therapeutically effective amount, will be an amount of the S-CDS sufficient to produce a beneficial CNS-related anti-inflammatory effect while maintaining acceptably low peripheral anti-inflammatory steroid levels.

The precise amount of the S-CDS in the complex present in the transmucosal dosage form will vary with the particular S-CDS-cyclodextrin complex selected, the weight and condition of the subject to which the dosage form is administered, the type of transmucosal dosage form selected and the medical condition for which the dosage form is administered. Because of the need for maintaining relatively low peripheral levels of steroids, the instant dosage forms are administered more frequently, but in much smaller dosage amounts, than would be expected based on prior art teachings.

Furthermore, one of skill will appreciate that the therapeutically effective amount of the S-CDS administered herein may be lowered or increased by fine tuning and/or by administering the S-CDS according to the invention with another active ingredient. The invention therefore provides a method to tailor the administration/treatment to the particular exigencies specific to a given mammal. Therapeutically effective amounts may be easily determined, for example, empirically by starting at relatively low amounts and by step-wise increments with concurrent evaluation of beneficial effect.

In the case of the estrogen-CDSs, for example the representative E₂-CDS, for use in perimenopausal or postmenopausal women, a suitable buccal dosage form comprises an anhydrous formulation comprising a substantially saturated complex of the estrogen-CDS in the selected cyclodextrin in which from about 0.5 to about 2.0 mg of the estrogen-CDS, such as E₂-CDS, is present. Such an estrogen-CDS can be administered per day or every other day to alleviate post menopausal symptoms, especially vasomotor symptoms such as hot flashes/hot flushes, vaginal atrophy, vaginal dryness/lack of lubrication, night sweats, insomnia, depression, nervousness, urinary incontinence, irritability and anxiety; to treat symptoms of female sexual dysfunction, particularly that comprising hypoactive sexual desire type female sexual dysfunction or sexual pain type female sexual dysfunction; or for treating or slowing/hindering the development of osteoporosis or of cognitive impairment, such as, for example, Alzheimer's disease, particularly when treatment is initiated early in the peri- or early postmenopausal period. This dose is as small as about 0.01 mg/kg or lower. The dosage amount and frequency is controlled so that one or more of these symptoms is/are diminished while the average steady-state peripheral estradiol levels are not elevated to above about 50-60 pg/mL; in the case of an estrogen-CDS such as E₂-CDS, for use in perimenopausal or postmenopausal women, these are considered acceptably low peripheral estrogen levels. Preferably, the average steady-state peripheral estradiol levels are not elevated above about 40 pg/mL, even about 20 pg/mL or lower, and/or with average peak estradiol peripheral levels (which are reached shortly after administration) in such women preferably not above about 70-90 pg/mL or even lower.

In the case of women who are in their reproductive years, and in whom chronic reduction of gonadotropin secretion for fertility regulation (contraception) or weight control or treatment of gonadal steroid-dependent diseases, such as endometriosis, are desired, higher peripheral steroid levels are still considered sufficiently low for purposes of this invention, for example as is normal in younger females; nevertheless, a similar daily dosage of from about 0.5 to about 2 mg of the estrogen-CDS in a buccal dosage form is envisioned.

In the case of estrogen-CDSs, for example E₂-CDS, for use in the human male, a suitable buccal dosage form comprising an anhydrous formulation of a substantially saturated complex of an estrogen-CDS such as E₂-CDS in the selected cyclodextrin in which from about 0.01 to about 0.5 mg per day of the estrogen-CDS is present can be administered for such period of time as required until symptoms diminish, for example approximately 2 to 7 days in men, with resumption of daily or every other day dosing when symptoms recur, to alleviate symptoms of male sexual dysfunction such as erectile dysfunction, male orgasmic disorder, inhibited or hypoactive sexual desire and priapism. Assuming approximately 30% bioavailability, this buccal dose calculates to an actual usable dose of only about 0.003 to about 0.015 mg per day, which divided by an average 70-80 kg weight, gives an approximate 0.0000375 to 0.00021 or less mg/kg dose in men. In any event, dosage amounts and dosage frequencies are such that they will not substantially elevate average peripheral estradiol levels to above normal levels in the male, i.e., they will not elevate average peripheral estradiol levels more than about 10-15% above normal levels. These are what are generally considered acceptably low peripheral estrogen levels in the male. This in turn will prevent peripheral estradiol levels from inhibiting ejaculation, so that both proceptive and consummatory aspects of male sexual behavior will be improved.

In the case of an estrogen-CDS such as E₂-CDS for treatment of prostate cancer, where the desire to keep peripheral estradiol levels low needs to be balanced against the need to treat a serious, potentially life-threatening illness, higher doses such as about 0.5 mg per day, administered more frequently, such as daily, may be acceptable.

In one particular aspect, there is provided herein a buccal tablet, buccal wafer or buccal patch comprising an anhydrous formulation of a substantially saturated complex of the compound 17β-[(1-methyl-1,4-dihydro-3-pyridinyl)carbonyloxy]estra-1,3,5(1)-trien-3-ol, i.e. E₂-CDS, with a hydroxyalkyl, carboxyalkyl or carboxymethylethyl derivative of β- or γ-cyclodextrin comprising from about 0.01 to about 2.0 mg of said compound and a buccally acceptable vehicle therefor. The buccal dosage forms comprising from about 0.01 up to but not including 0.5 mg of E₂-CDS are primarily designed for use in men, while those comprising from about 0.5 to about 2.0 mg of E₂-CDS are primarily designed for use in women. The buccal dosage forms in which the cyclodextrin is hydroxypropyl-β-cyclodextrin such as 2-hydroxypropyl-β-cyclodextrin), hydroxypropyl-γ-cyclodextrin (such as 2-hydroxypropyl-γ-cyclodextrin), hydroxyethyl-β-cyclodextrin, hydroxyethyl-γ-cyclodextrin, carboxymethyl-β-cyclodextrin, carboxymethyl-γ-cyclodextrin, carboxyethyl-β-cyclodextrin, carboxymethyl-γ-cyclodextrin or carboxymethyethyl-β-cyclodextrin are of particular interest.

As used herein, “treating” means reducing, preventing, hindering the development of, controlling, alleviating and/or reversing the symptoms in the individual to which a compound of the invention has been administered, as compared to the symptoms of an individual not being treated according to the invention. A practitioner will appreciate that the complexes, compositions, dosage forms and methods described herein are to be used in concomitance with continuous clinical evaluations by a skilled practitioner (physician or veterinarian) to determine subsequent therapy. Such evaluation will aid and inform in evaluating whether to increase, reduce or continue a particular treatment dose, and/or to alter the mode of administration.

The methods of the present invention are intended for use with any subject/patient that may experience the benefits of the methods of the invention. Thus, in accordance with the invention, the terms “subjects” as well as “patients” or “female mammal” include humans as well as non-human subjects, particularly domesticated animals (domestic and farm animals), zoo animals and rare or endangered or expensive mammalian species.

The expression “female sexual dysfunction” as used herein includes four broad categories: sexual desire disorders, sexual arousal disorders, orgasmic disorders, and sexual pain disorders; of these four, the most common is hypoactive (inhibited) sexual desire disorder (HSDD). HSDD is defined as persistent or recurrent deficiency (or absence) of sexual fantasies, thoughts and/or desire for, or receptivity to, sexual activity, which causes personal distress. HSDD can result from, among other etiologies, physical illness, hormonal abnormality, or medications that affect libido. In postmenopausal women, sexual dysfunction may be closely linked to and include symptoms associated with the estrogen deprivation of menopause, such as vaginal dryness/lack of lubrication and consequent pain associated with intercourse, which can be closely associated with diminished sexual desire. Other postmenopausal symptoms such as night sweats, hot flushes, insomnia, depression, nervousness, urinary incontinence, irritability and anxiety are also likely to be associated with diminished sexual desire.

The expression “male sexual dysfunction” includes, in the main, erectile dysfunction, male orgasmic disorder, inhibited or hypoactive sexual desire and priapism. Inhibited or hypoactive sexual desire refers to a decrease in desire for, or interest in, sexual activity and can result from a variety of causes, including physical illness, depression, hormonal abnormality or medications that affect libido.

Male sexual behavior is composed of proceptive and consummatory behaviors. The proceptive behaviors include the awareness of the presence of a receptive female, the pursuit of that female and the positioning of the body (mounting) to allow insertion of the penis into the vagina. This later behavior, turned intromission, as well as its prerequisite erection of the penis and eventual ejaculation, are the consummatory components of masculine sexual behavior. The accomplishment of ejaculation requires the entire repertoire of the aforementioned behavior. Treating dysfunction of proceptive behavior only is not sufficient when dysfunction of consummatory behavior also exists.

The expression “peripheral estradiol levels” as used herein refers to serum estradiol levels obtained throughout the treatment period, using repeated dosing on a once per day or every other day schedule. The expression “steady-state peripheral estradiol levels” as used herein refers to serum estradiol levels obtained throughout the treatment period, using repeated dosing on a once per day or every other day schedule, excluding initial peak levels obtained within about 1-2 hours after the initial dose.

In the case of androgen-CDSs, for example, the representative T-CDS,, a suitable buccal dosage form comprises an anhydrous formulation of a substantially saturated complex of T-CDS₁ or other androgen-CDS in the selected cyclodextrin in which from about 1.0 to about 5.0 mg of the selected androgen-CDS, for example, T-CDS₁, is present; the same or lower dosages may be appropriate for administration to females in order to minimize side-effects. These dosages are extremely low compared to the about 28 mg/kg dosages of T-CDS, previously given to female rats; see Bodor et al. J. Pharm. Sci., Vol. 73, No. 3, 385-389 (March 1984); and also very low compared to the 11.9 mg/kg administered intravenously in HPβCD to castrated male rats described in Bodor U.S. Pat. No. 5,017,566. Such an androgen-CDS can be administered at a total daily dose of from about 1 to about 15 mg/day, preferably from about 3 to about 8 mg/day, in the treatment of hypogonadism, cryptorchidism, the male climacteric, breast engorgement, cancer of the female breast and dysmenorrhea. In the case of the androgen-CDSs, “acceptably low peripheral steroid levels” are amounts which do not produce significant peripheral androgenic side-effects such as impotence and azoospermia in the male and masculinization in the female, or about 150 ng/mL or less of plasma testosterone.

In the case of anti-inflammatory steroid-CDSs, for example, the representative DEX-CDS, a suitable buccal dosage form comprises an anhydrous formulation of a substantially saturated complex of the anti-inflammatory steroid-CDS such as DEX-CDS in the selected cyclodextrin in which from about 2.5 to about 20 mg of DEX-CDS or other suitable anti-inflammatory steroid-CDS is present. Such an anti-inflammatory steroid-CDS can be administered daily or every other day in the treatment of brain inflammation and edema, for example after brain surgery or in the case of traumatic brain injury or a brain tumor. If the patient is not able to use a buccal tablet for a period of time after surgery or is unconscious for other reasons, a buccal patch (in which the drug is released only from the side which adheres to the buccal mucosa while the other side is non-permeable) may be used to deliver the drug. Thus, a sustained release buccal patch is used in such patients in order to administer from about 5 to about 20 mg of the drug over an extended period of time, such as twenty-four hours. These dosages are extremely low compared to those described in the literature, i.e. 10 mg/kg to adult male rats, as described by Anderson et al., Neuroendocrinology, 50, 9-16 (1989). In the case of the anti-inflammatory steroid-CDSs, “acceptably low peripheral steroid levels” are amounts which do not produce significant glucocorticoid peripheral side-effects such as hepatocyte hypertrophy, Addison's disease-like syndromes, hepatomegaly, hepatocellular degeneration and necrosis.

Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### EXAMPLES

### Example 1

Phase Solubility Study

A phase solubility study is carried out as follows. Excess S-CDS (E₂-CDS, DEX-CDS or T-CDS₁) in a small amount of ethanol is added to cyclodextrin solutions of various concentrations of hydroxypropyl-β-cyclodextrin (HPβCD), hydroxypropyl-γ-cyclodextrin (HPγCD) or carboxymethylethyl-β-cyclodextrin and allowed to complex as described in Example 2, 3 or 4 below. Excess, undissolved S-CDS, if any, is removed by filtration. The amount of S-CDS in the complex is measured to obtain a data point. This process is repeated with different known concentrations of cyclodextrin until several data points are obtained. These data points are then plotted graphically, each data point representing the maximum amount of the selected S-CDS that can be complexed with a specific concentration of the selected cyclodextrin, i.e. each point represents a saturated S-CDS/cyclodextrin complex. Points on the line generated by the data points represents HTA ratios. Any point on the line represents a specific, unique saturated S-CDS/cyclodextrin complex. One of skill in the art will realize that the same results will be generated if excess cyclodextrin is added to S-CDS solutions of known concentrations.

When the molar concentrations of the selected S-CDS to those of the selected cyclodextrin are plotted and presented graphically, the plotted lines represent the maximal solubilization of the drug for the conditions tested, that is, the HTA ratio of the concentration of the selected S-CDS to the concentration of the selected cyclodextrin. The area above each of the plotted lines represents conditions where excess insoluble S-CDS is present. The area below each of the plotted lines represents the conditions where cyclodextrin is in excess of the amount needed to maintain the complex in solution.

The plot will also show how much additional cyclodextrin is needed to maintain a specific amount of the drug is its saturated complex. The plot also, by the slope of the line, can indicate whether a 1:1 complex or a 1:2 complex of drug to cyclodextrin is formed, i.e., whether one molecule of the drug is complexed with one molecule of cyclodextrin (1:1 complex) or whether one molecule of the drug is complexed with 2 molecules of the cyclodextrin (1:2 complex), in which case 2 molecules of the cyclodextrin essentially surround and protect the drug molecule.

In the case of E₂-CDS and hydroxypropyl-β-cyclodextrin (HPβCD) at high cyclodextrin concentrations, the complex is largely a 1:2 complex. The two molecules of HPβCD are believed to hydrogen-bond to each other at high cyclodextrin concentration and incorporate in the cavity between them the E₂-CDS molecule. This is thought to be a stepwise process, in which the 1:1 complex first forms, then a second HPβCD molecule H-bonds with the HPβCD in the 1:1 complex, forming the 1:2 complex. Of course, frequently a mixture of 1:1 and 1:2 complexes will be obtained, but a predominance of the 1:2 complex is advantageous. Since the 1:2 complex formed at higher concentrations of HPβCD is a stronger complex than a 1:1 complex, the E₂-CDS in the saturated solution formed when such a 1:2 complex releases the drug in the body fluid at the mucosa is even more unstable, i.e. has even higher thermodynamic activity, than the E₂-CDS released from a 1:1 complex, favoring even greater movement of the drug through the mucosa. The case of E₂-CDS with HPγCD is similar.

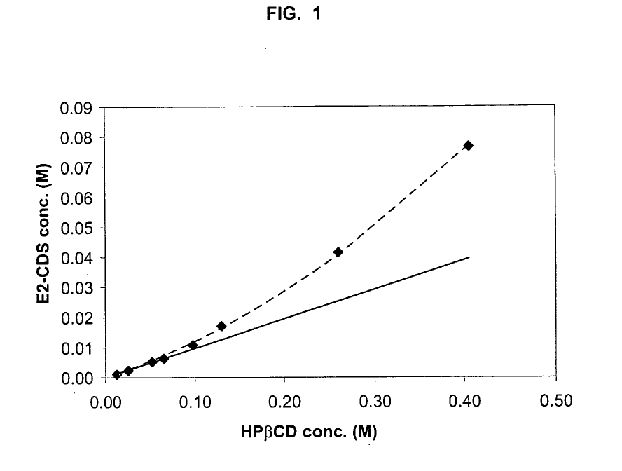

FIG. 1 is a representative phase solubility diagram for E₂-CDS and hydroxypropyl-β-cyclodextrin. This is based on data contained in Bodor U.S. Pat. No. 5,017,566 and Brewster et al., J. Pharm. Sci., 77, 981-985 (1988). The data points are 6.73 mg/mL at 20% w/v and 16.36 mg/mL at 40% w/v (both from Brewster et al.) and 30.19 mg/mL at 60% w/v (from the patent). All data are for HPβCD having an average 7 degrees of substitution and were converted to a molar scale using appropriate molecular weights.

More commonly, a phase solubility diagram for E₂-CDS or other S-CDS can be obtained by stirring a suspension of excess E₂-CDS into various concentrations of the selected cyclodextrin in water. Sometimes, a small amount of ethanol can be used to accelerate dissolution.

### Example 2

Preparation of Approximately 3% Complex of E₂-CDS with HPβCD

Dissolve 232 g of 2-hydroxypropyl-β-cyclodextrin (HPβCD) (Cerestar, degree of substitution 4.5) in deionized 465 mL water (ASTM Type I) to form an approximately 33% w/v solution. Adjust the pH to 8.4-9.6 with sodium carbonate 1% solution. Degas the solution by passing argon through it. Add slowly, drop-wise, under stirring and bubbling argon, at 20-25° C., a solution of E₂-CDS (7.5 g) in ethanol (188 mL). Allow time after each addition for the solution to become clear. The addition takes about 4 hours and it is slower at the end. A clear solution will result. Evaporate the solution to dryness in a rotary evaporator (bath temperature 35° C.). Reconstitute the residue in water, calculated to obtain the initial concentration of the cyclodextrin solution. Filter the solution through a 47 mm, 0.45 μm nylon 66 membrane filter, while covering with argon. Freeze-dry the filtrate, grind the resulting solid in a blender and pass it through a 60 mesh sieve. The resulting complex, an off-white amorphous solid (˜233 g), is transferred in a jar and analyzed. The complex should contain about 29-32 mg E₂-CDS per gram. E₂-CDS should have a chromatographic purity of at least 97% by HPLC. The yield of complexation (based on E₂-CDS) should be 82-96%.

Following the above procedure but substituting an equivalent quantity of T-CDS, or DEX-CDS for the E₂-CDS above affords a freeze-dried amorphous complex of T-CDS, or DEX-CDS, respectively, with HPβCD.

### Example 3

Preparation of Approximately 2.5% Complex of E₂-CDS with HPγCD

Dissolve 45 g of 2-hydroxypropyl-γ-cyclodextrin (HPγCD) (Wacker, Cavasol W8 HP) in deionized 135 mL water (DIUF) to form an approximately 25% w/v solution. Adjust the pH to 8.4-9.6 with sodium carbonate 1% solution. Degas the solution by passing argon through it. Add slowly, drop-wise, under stirring and bubbling argon, at 20-25° C., a solution of E₂-CDS (1.5 g) in ethanol (3 mL). Allow time after each addition for the solution to become clear. The addition takes about 4 hours and it is slower at the end. A clear solution will result. Evaporate the solution to dryness in a rotary evaporator (bath temperature 35° C.). Reconstitute the residue in water, calculated to obtain the initial concentration of the cyclodextrin solution. Filter the solution through 47 mm, 0.45 μm nylon 66 membrane filter, while covering with argon. Freeze-dry the filtrate, grind the resulting solid in a blender and pass it through a 60 mesh sieve. The resulting complex, an off-white amorphous solid (˜42 g), is transferred in a jar and analyzed. The complex should contain about 20-25 mg E₂-CDS per gram. E₂-CDS should have a chromatographic purity of at least 97% by HPLC. The yield of complexation (based on E₂-CDS) should be 82-96%.

Following the above procedure but substituting an equivalent quantity of T-CDS₁ or DEX-CDS for the E₂-CDS used above affords a freeze-dried amorphous complex of T-CDS₁ or DEX-CDS, respectively, with HPγCD.

### Example 4

Preparation of Complex of E₂-CDS with CMEβCD Method 1:

Dissolve 100 mg of E₂-CDS and 500 mg of O-carboxymethyl-O-ethyl-β-cyclodextrin (CMEβCD) in 10 mL of ethanol and sonicate the solution for 1 hour. Then remove the solvent, reconstitute the residue with water, filter and lyophilize. The complex should contain about 25 mg E₂-CDS/g.

Method B:

Dissolve 2 g of CMEβCD in 20 mL of 0.10M pH 9.0 borate buffer. Adjust the pH with 1N sodium hydroxide solution. Then dissolve 150 mg of E₂-CDS in 2 mL of ethanol and add the resultant solution to the cyclodextrin solution. Stir for 3 hours at 0° C. under argon, remove the solvent in vacuo, reconstitute the residue with pH 9 borate buffer and lyophilize.

The foregoing methods can be adapted to provide similar complexes of other steroid-CDSs with CMEβCD, such as, for example, DEX-CDS and T-CDS₁.

### Example 5

Manufacture of Buccal Tablets for Clinical Trails

In accord with the invention, a buccal tablet was designed for use in clinical trials to deliver E₂-CDS transmucosally and thus avoid the instability of E₂-CDS in gastrointestinal fluid, which leads to multiple decomposition productions starting with water addition and/or oxidation, as well as hepatic first pass metabolism. Transmucosal absorption is highly effective from the invention's saturated complex of E₂-CDS in HPβCD (as prepared, for example, in Example 2 above) with minimal additives. A placebo was also prepared for the clinical trials.

FORMULATION

| | | | E₂-CDS | | E₂-CDS | | E₂-CDS | |
|---|---|---|---|---|---|---|---|---|
| | Placebo | | 0.5 mg | | 1.0 mg | | 2.0 mg | |
| E₂-CDS/ | 0.00 | | 16.67 | | 33.33 | | 66.67 | |
| HPβCD | | | | | | | | |
| complex | | | | | | | | |
| HPβCD | 33.33 | | 0.00 | | 0.00 | | 0.00 | |
| (freeze-dried) | | | | | | | | |
| Sorbitol | 62.67 | | 82.33 | | 65.67 | | 32.33 | |
| powder NF | | | | | | | | |
| Magnesium | 1.00 | | 1.00 | | 1.00 | | 1.00 | |
| stearate NF | | | | | | | | |
| Opadry Yellow | 3.00 | | 0.00 | | 0.00 | | 0.00 | |
| 31F22300 | | | | | | | | |
| | 100.00 | mg | 100.00 | mg | 100.00 | mg | 100.00 | mg |

Similar buccal tablets can be prepared containing other steroid-CDSs such as DEX-CDS or T-CDS, and/or other cyclodextrins such as HPγCD, CMEβCD or other cyclodextrin identified in this specification.

Investigation of Female Rat Sexual Behavior After Ovariectomy

Rationale

Castration causes the termination of sexual behavior in rats, but the sexual activity of castrated female rats can be reestablished by administration of estradiol.

In female rats, estradiol acts in the hypothalamus and preoptic area to regulate the expression of lordosis, an important component of female reproductive behavior and a characteristic posture of the female for a sexually active male to allow copulation. The expression “lordosis” as used herein refers to vertebral dorsiflexion performed by female quadrupeds in response to adequate stimuli from a reproductivity competent male. Estradiol acts on multiple molecular targets that may converge on common biochemical pathways to ensure integration of sensory and neurochemical cues that regulate lordosis expression. Thus, lordosis was selected as an indicator of restoration of female sexual function in ovariectomized female rats and an appropriate indicator for alleviating symptoms of female sexual dysfunction.

Circulating luteinizing hormone (LH) is a biomarker reflecting the CNS effects of estradiol. Estrogen diminishes the secretion of luteinizing hormone-releasing hormone (LHRH) and hence reduces the secretion of LH. Therefore, LH and estradiol levels were investigated to measure the central and peripheral effects of E₂-CDS, respectively.

Experimental Design

Adult female Sprague Dawley rats (220-250 g) from Charles River Hungary Ltd., Godollo, Hungary, were used. Animals were kept in community cages (4 animals/cage) in a climate-controlled room (23∀2EC, 50-60% humidity), with a 14 hour light, 10 hour dark cycle of artificial lighting, using reversed light/dark cycle. Food and water were available ad libitum.

After a minimum five-day acclimatization period, animals were ovariectomized under ether anesthesia, then were left to recover for 3 weeks before testing (reconvalescence). All animals were treated in accordance with the guidelines of the European Communities Council Directive (86/609/EEC) and studies were permitted by the Institutional Animal Care Commission.

Estradiol benzoate and progesterone were obtained from Sigma Chemical Co. Inc., Budapest, Hungary. 2-Hydroxypropyl-β-cyclodextrin was purchased from Cerestar Inc., Hammond, Ind., US. Estradiol benzoate was dissolved in 40 w/v % 2-hydroxypropyl-β-cyclodextrin (HPβCD) solution and diluted with 27 w/v % HPβCD solution (0.29 mg/kg is equimolar to that of 0.3 mg/kg E₂-CDS). E₂-CDS as a 3% complex with HPβCD (E₂-CDS-CD) was dissolved in distilled water and diluted with 27% HPβCD solution. E₂-CDS-CD was synthesized by Alchem Laboratories Corporation, Alachua, Fla., US, using the procedure of Example 2 above.

Behavioral Testing

After recovery from surgery, ovariectomized female rats were divided into four groups and treated once a day for five days intravenously, via a bolus injection through the tail vein, as follows: (1) control, 27% HPβCD solution; (2) 0.003 mg/kg E₂-CDS dissolved in 27% HPβCD solution; (3) 0.01 mg/kg E₂-CDS dissolved in 27% HPβCD solution; and (4) 0.03 mg/kg E₂-CDS dissolved in 27% HPβCD solution. A minimal number of ovariectomized (8 to 12) females were used per group. Intravenous treatments either with E₂-CDS or HPβCD (controls) were carried out daily for 5 days beginning 2 days prior to the first day of behavior observations, in a volume of 0.05 mL/100 g body weight.

The investigation of estradiol benzoate (EB) was performed in newly randomized previously ovariectomized females after a resting period of 3 weeks. Animals (7 to 11 per group) were treated with 0.003, 0.01 and 0.03 mg/kg estradiol benzoate intravenously once a day for 5 consecutive days similarly to the protocol applied for E₂-CDS. Estradiol benzoate was dissolved in 40% HPβCD and diluted with 27% HPβCD solution (0.29 mg/kg stock solution equimolar to that of E₂-CDS).

The behavior test was conducted in a plexiglass observation cage during the dark cycle. During behavioral observations, only a dim red light was on.

An experienced and active male rat was placed in the arena 5 minutes prior to the female. Each female was observed for the time of ten successful mounts per test session or for a maximum of 10 minutes, and the number of lordosis responses was recorded. The lordosis quotient (LQ) expresses the estrogen effect on sexual receptivity and was calculated as follows:
LQ=100×number of lordoses/10 mounts
The observation of the sexual behavior of each female was carried out, in the case of E₂-CDS, every day for 22 days; in the case of EB, investigations were carried out every day for 10 days. On days 0, 3, 7, 10, 12, 15 and 18, blood samples were taken to determine levels of LH and estradiol. Citrated blood samples were taken by retro-orbital sinus puncture under light ether anesthesia. The samples were stored at 4EC for one hour, then centrifuged at 1000 g for 10 minutes. Plasma was separated and stored at −80EC until assayed. Plasma LH concentrations from individual samples were measured by double antibody radioimmunoassay kits obtained from Amersham Pharmacia Biotech, Rome, Italy. Plasma estradiol levels were determined by double antibody I¹²⁵ isotope-RIA kits obtained from BioChem Immuno System. The limit of detection was 15 pg/mL.

Behavioral changes were analyzed using the Mann-Whitney U test (Siegel, Nonparametric Statistics for the Behavioral Sciences, New York; McGraw-Hill Book Company, Inc., 1956). The Fisher exact test was used for percentage comparisons (Zar, Biostatistical Analysis, Prentice Hall, Inc., Englewood Cliffs, N.J., 1974). Serum LH data were analyzed for each time and treatment group by analysis of variance (ANOVA) followed by Bonferroni posthoc test. Plasma LH and estradiol concentrations were evaluated by the computerized standard curve program of Prism software (Version 3.0, Graph Pad, San Diego, Calif., US).

Results

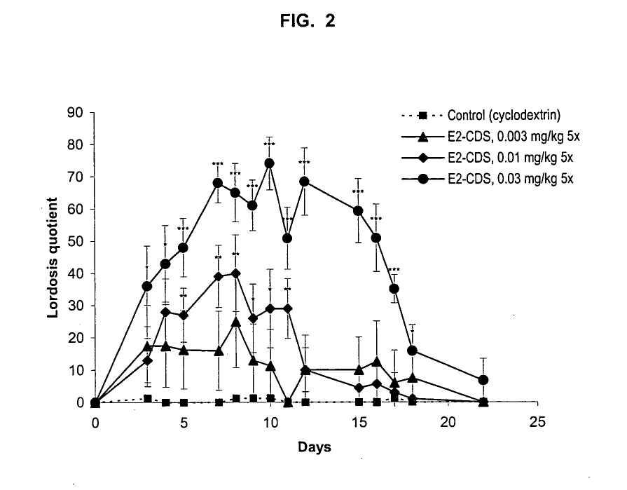

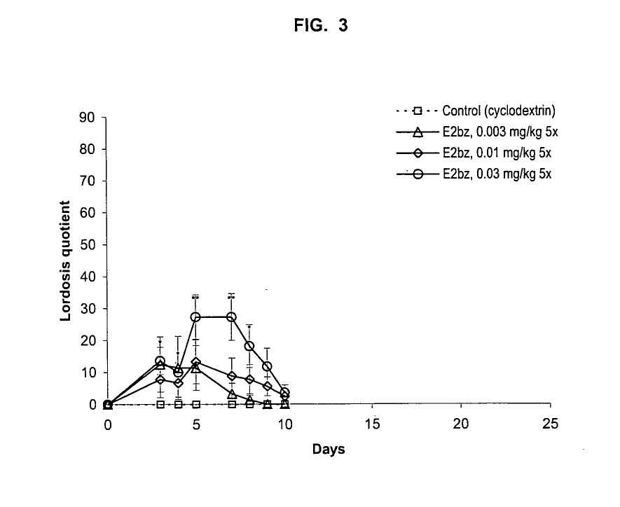

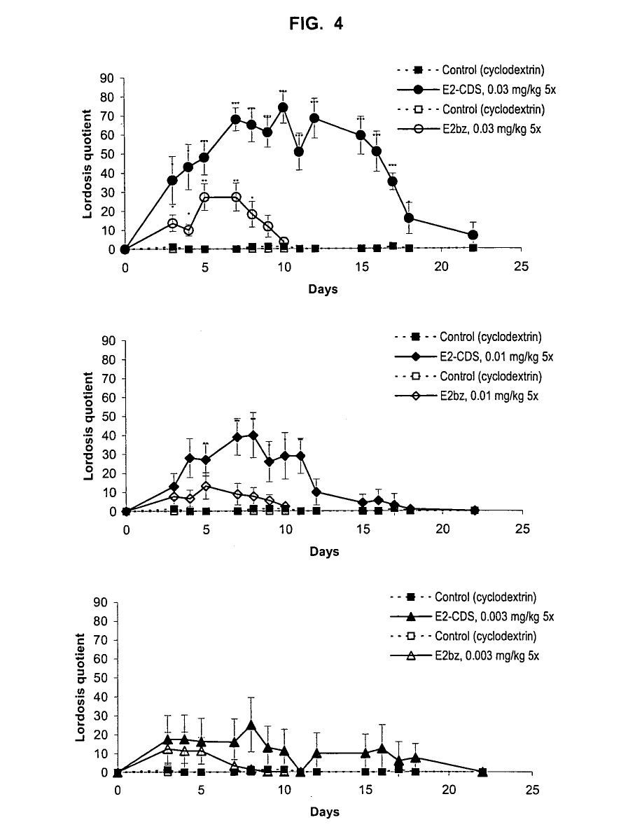

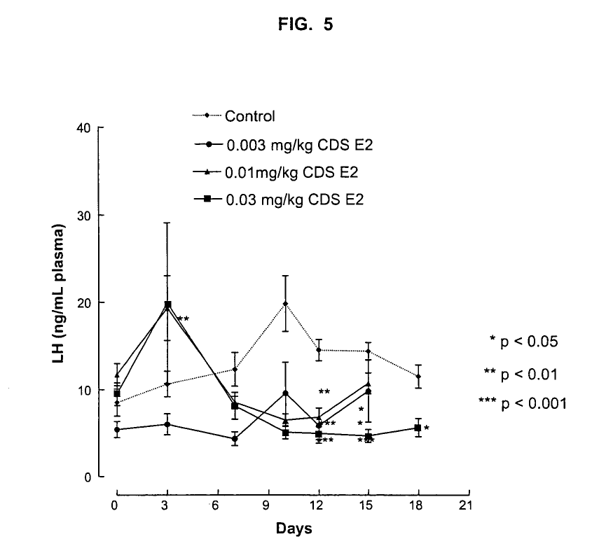

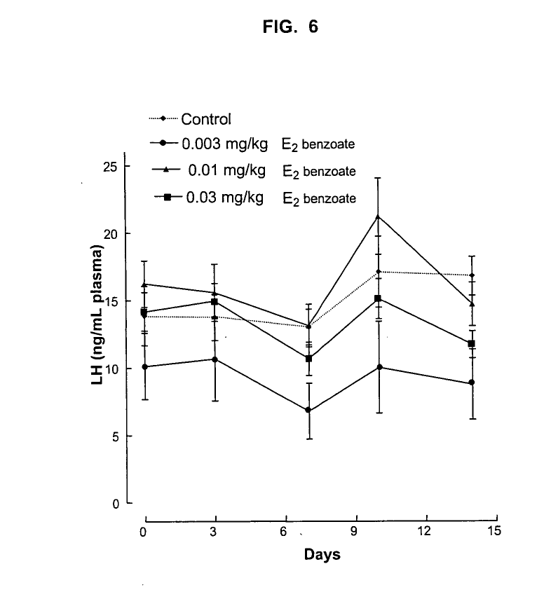

FIGS. 2-6 show the results obtained. In FIG. 2, data are mean ∀SE for 8-12 animals per group; *p<0.05, **p<0.01, ***p<0.001 using the Mann-Whitney U test. In FIG. 3, data are mean ∀SE for 7-11 animals per group, with *, ** and *** as defined for FIG. 2. The data presented in FIG. 2 and FIG. 3 are reorganized in FIG. 4 so as to more readily compare the effect of the same dose of E₂-CDS and estradiol benzoate (E₂-Benz). In FIGS. 5 and 6, data are mean ∀SE for 7-12 animals per group, *p<0.05, **p<0.01, ***p<0.001 using ANOVA followed by the Bonferroni posthoc test.

At the dose of 0.03 mg/kg, the lordosis quotient LQ was significantly enhanced by both E₂-CDS and estradiol benzoate. In the case of E₂-CDS, this effect lasted from day 3 to day 18, as shown in FIG. 2. The effect from estradiol benzoate was less pronounced and lasted only from day 3 to day 8; see FIG. 3. As also seen in FIG. 3 as well as the first portion of FIG. 4, the LQ value for estradiol benzoate was about three times lower than that obtained for E₂-CDS: the maximal values of LQ after E₂-CDS and estradiol benzoate treatments were 73 and 27.3 respectively.

At the dose of 0.01 mg/kg, E₂-CDS significantly enhanced the LQ from day 5 to day 11. The increase, although thereafter not statistically significant, lasted till day 15. This dose of estradiol benzoate slightly increased the LQ from day 3 to day 10 (about 3 times less compared to E₂-CDS), but this effect was not statistically significant. See FIGS. 2, 3 and the second portion of FIG. 4.

At the dose of 0.003 mg/kg, doses of the test compounds slightly enhanced the lordosis quotient, but these effects were not statistically significant (estradiol benzoate, days 3-7; E₂-CDS, days 3-18). See FIGS. 2, 3 and the third portion of FIG. 4.

FIG. 5 shows that plasma LH levels were suppressed at all dosage levels of E₂-CDS tested, i.e. at 0.003, 0.01 and 0.03 mg/kg. Even at the low i.v. dose of 0.03 mg/kg, the plasma LH level was suppressed in a statistically significant manner for up to 18 days; plasma LH suppression lasted for up to 15 days even for the very low dose of 0.003 mg/kg. In contrast, as shown in FIG. 6, none of the tested dosages of estradiol benzoate gave statistically significant LH suppression.

The foregoing studies show that E₂-CDS can restore female sexual function in rats and indicate that symptoms of female sexual dysfunction can be alleviated through its administration to females, including women, at doses far lower than previously thought possible, while maintaining appropriate peripheral levels of estrogen.

Clinical Studies

Recently, E₂-CDS has been studied in clinical trials of postmenopausal women given a single 2.5 mg or 5 mg dose of E₂-CDS administered buccally. Even more recently, in a Phase I clinical study of postmenopausal women, two different administration regimens of a 2.86 mg E₂-CDS buccal delivery tablet were evaluated for safety and effects on hormone levels. The subjects were 12 healthy postmenopausal volunteers, divided into two groups of six. In Group A, women were dosed once daily for 10 days (10 doses); in Group B, women were dosed once every other day for 13 days (7 doses). In both groups, measurements of serum total and free estradiol, estrone, LH, FSH, prolactin, SHBG and testosterone were made at certain intervals throughout the treatment period and also at 72 hours after the last dose and levels of urinary estrone and the ratio of 2OHE₁/16OHE₁ on Day 1 and 72 hours after the last dose were determined, too. A brief evaluation of the results follows:

Results

1. Dissolution

E₂-CDS was administered in a buccal delivery form (a buccal tablet) as a saturated complex with hydroxypropyl-β-cyclodextrin. The median buccal dissolution time (and “buccal residence time”) was 11 minutes and 13 seconds (minimum 1.12 min.sec, maximum 23.03 min.sec). This dissolution time is convenient for patients.

2. Estradiol (E₂)

During the first 24 hours after the administration of 2.86 mg E₂-CDS, the maximum concentration (Cₘₐₓ) of E₂ in serum was 102±20.2 pg/mL (with subject 12, who subsequently showed much higher levels than all other subjects), and this peak was reached at 1.2±0.4 hours. The Cₘₐₓ without subject 12 was 97.8±20.0 pg/niL. The average Cₘₐₓ in the earlier clinical trial, which used a buccal delivery form with an average 45 minute dissolution rate, was 153.4 pg/mL after 2.5 mg E₂-CDS, with Tₘₐₓ of 2 hours. One explanation for this difference might be the difference in the dissolution (and buccal residence) time of the two formulations used in these two different studies.

Neither of the administration regimens (once daily, versus once every other day) resulted in an accumulation, i.e. increase, in the trough serum estradiol levels (CTR), measured always before the next consecutive dose of E₂-CDS. However, the serum levels that were established during the repeated dosing were different between the two administration regimens. At steady state E₂ CTRₘₐₓ of 95.3±76.6 pg/mL was reached with the daily administration (if values for subject 12 are omitted, this concentration is 65.2±23.2 pg/mL). The steady state CTRₘₐₓ serum concentration of E₂ was 26.4±9.8 pg/mL with the every other day administration regimen.

The post-study (72 hours after the last dose) E₂ concentration was 11.5±2.7 pg/mL in the every other day group, and 36.8±54.6 pg/mL in the once daily group, respectively. In the once daily group, this post-study value would be 12.5±6.5 pg/mL if the values for subject 12 are omitted.

3. Estrone (E₁)

E₁ was measured during the first 24 hours along with E₂ and at post-study (i.e. 72 hours after the last dose). The post-study values were 47.5±49.7 pg/mL (without subject 12: 27.8±12.8 pg/mL) and 31.4±9.4 pg/mL in the once daily, and in the every other day dosing regimen group, respectively. During repeated administration a similar trough level pattern to E₂ without accumulation can be anticipated for E₁ as well in both dosing regimen groups, i.e. a steady state at somewhat higher level for the once daily administration group, than for the once every other day group.

4. LH Suppression

During the first 24 hours the maximum decrease in LH was 13.8±4.9 and 12.7±6.8 mIU/mL from baseline in Group A and B, respectively (Group A showed slightly higher baseline values). This corresponds to a 35-40% decrease in LH levels from baseline. The maximum LH depression occurred at 7.3±5.3, and 7.3±2.7 hours post-dose in Group A and B, respectively. At post-study (72 hours after the last dose) LH levels were not different any more from screening/baseline values. Though the 24-hour LH suppression profile was determined only during the first 24 hour post-dose period, a similar daily LH suppression pattern can be anticipated on each dosing day. Blood samples are available for additional pre-dose LH measurements for days 3-11 in group A (once daily), and for days 3, 5, 7, 9, 11, 13, in group B (every other day), respectively.

5. FSH Suppression

A 15 and 16% suppression in FSH levels was observed during the first 24 hours post-dose in Group A and B, respectively. The maximum suppression occurred at 13.2±5.7, and 11.8±5.8 hours post-dose in Group A and B, respectively. In contrast to LH, post-study FSH levels were still below the screeningibaseline values (by 14-25%). The kinetics of FSH suppression seems to be different from that of LH suppression: it develops more slowly after the administration of E₂-CDS, and FSH remains somewhat suppressed throughout the entire length of the study, even at 72 hours after the last dose. Blood samples for additional pre-dose hormone level measurements are available for days 3-11, and 3, 5, 7, 9, 11, 13, in group A and B, respectively. The extent of maximum FSH suppression (12.5%) during the first 24 hour post-dose in the previous clinical study after 2.5 mg E₂-CDS was similar to the extent in this study, having in mind the slightly higher dose (2.86 mg) administered in the second study.

6. Prolactin

Mean baseline concentrations of prolactin were higher in Group A than in Group B. Likewise, there were higher mean concentrations on day 13 in Group A than on day 16 in Group B. The increase in prolactin levels compared to baseline was 29.8 and 16%, in Group A and B, respectively, by the end of the study. The differences between the two groups were not statistically significant.

7. SHBG (Sex Hormone-binding Globulin)

SHBG concentrations in Group A (day 13) and Group B (day 16), respectively, were by 22.2 and 41.2% higher than at baseline on day 1. Statistical differences between the groups were not demonstrated.

8. Testosterone

Serum concentrations of testosterone on day 1 decreased both in Group A and B. Mean AUC₂₄ were by 31.1 and 23.0% lower than baseline AUC₂₄ (=C₀*24), in Group A and B, respectively. Serum testosterone concentrations on day 1 decreased to 1.3±1.9 and 4.0±3.9 ng/dL from 22.5±21.0 and 24.0±14.0 ng/dL, in Group A and B, respectively. The time to reach these minimum testosterone levels on day 1 were 6.5±11.7 and 7.3±11.3 hours in Group A and B, respectively. 72 hours after the last administered dose, testosterone levels returned and slightly exceeded those of baseline values by 14 and 28% in Group A and B, respectively. However, the differences between the two groups did not reach statistical significance in any parameter.

9. Urinary Estrone (E₁) and 2OHE₁/16OHE₁ (2-hydroxyestrone/16-hydroxyestrone)

Urine was collected for 24 hours on day 1 in both groups, and overnight (8 hours) on day 10 (Group A) and on day 13 (Group B), respectively, to determine the amounts of voided urinary estrone (E₁), 2OHE₁, 16OHE, and the ratios of 2OHE₁ to 16OHE₁. The mean amounts of E₁ and the ratios E₁/creatinine in the 24-hour urine on day 1 in both groups were very similar. Mean amounts and ratios of 2OHE1 to 16OHE₁ in 8-hour urine on day 10 in Group A appeared slightly higher than in Group B on day 13. Consequently, the differences of mean amounts (adjusted to an 8-hour urine collection period) and differences of mean ratios of day 10—1 in Group A were higher than the corresponding differences of day 13—1 in Group B (0.15 vs. 0.05). The difference of ratios approached statistical significance (p=0.077). On the last dosing day (day 10 in Group A, day 13 in Group B, respectively) mean amounts of 2OHE, were 4.46-times and 2.34-times higher than those values (adjusted to an 8-hour urine collection period) on day 1 in Group A and B, respectively. However, the increases in the amounts of urinary 16OHE₁ were only 2.48, and 1.26-times higher in Group A and B, respectively at the end of the treatment period compared to the day 18-hour adjusted values. During treatment the ratios of 2OHE₁ to 16OHE₁ increased by 63.6 and 54.7% in Group A and B, respectively.

10. Safety and Tolerance

There were seven adverse events (AEs) in total experienced by four subjects. The AEs were increased SGOT and CPK levels (1-1 case) headaches (2 cases) and 1-1 cases of glossitis, nausea and vomiting. All AEs were mild or moderate, no serious AE was observed. The relationship to trial drug was judged to be reasonably attributable in the single case of glossitis. All other AEs were considered as not reasonably attributable to the trial drug. The abnormal laboratory findings were a consequence of accidental injury and values returned to normal after 7 days. One AE (headache) required treatment with a single dose of 500 mg paracetamol. All AEs resolved without sequelae.

Conclusions:

The aim of this clinical study was to collect PK data on serum hormone levels (focus on serum E₂ concentrations) during a repeated administration study. 2.86 mg E₂-CDS was administered buccally once daily (group A), or once in every other day (group B). After reaching a steady state concentration (65.2±23.2 pg/mL without subject 12 in Group A and 26.4±9.8 pg/mL in Group B, respectively), trough E₂ levels did not increase with time, there were no signs of accumulation in either of the two groups. Based on a repeated measure ANOVA of E₂ trough concentrations that did not show a significant effect of time, or a subject*time interaction between days 7-11 in Group A, and including days 5, 7, 9, 11, and 13 in Group B, it can be concluded that the steady state E₂ trough concentrations were attained by day 5 and 7 in Group B and A, respectively. The attained steady state peripheral E₂ concentration in group A was stabilized in a range (65.2±23.2 pg/nl) where clinical efficacy, i.e. relief of vasomotor and urogenital symptoms would be expected. However, bearing in mind that the mechanisms of vasomotor symptoms are mostly CNS mediated, and also based on the preclinical observations that E₂ is trickled down from the brain as it is released from the inactive E₂Q⁺ precursor trapped behind the BBB, clinical efficacy is expected also in group B at lower peripheral trough E₂ levels. For practical reasons an every other day dosing regimen might be complicated for patients, however the once daily administration with lower doses (0.5, 0.75, 1.0, 1.25, 1.5, 1.75, 2.0 mg) should be sufficient to ameliorate postmenopausal symptoms, especially vasomotor and urogenital symptoms, and to effectively treat female sexual dysfunction, especially that involving deficiencies in sexual desire or sexual pain disorders. Further, a four week pack of tablets analogous to those typically used for dispensing estrogen/progestin combinations, e.g. Prempro®, or oral contraceptives, could be used in either case, with the alternate day regimen simplified for patients by alternating E₂-CDS buccal tablets with placebo tablets. The occurrence of few adverse events among which only one was judged as reasonably attributable to the trial drug proves the excellent safety and tolerance of E₂-CDS in the form of a buccally administered tablet. The finding of an increase in the urinary 2OHE₁ to 16OHE₁ ratio indicates a good safety profile in terms of breast cancer risk as well. Data from literature has consistently proven that a lower urinary 2OHE₁/16OHE₁ ratio represents an important biomarker for increased breast cancer risk. Treatment with E₂-CDS does not change the metabolism of E₂ and E₁ in a way that would confer an increased risk for breast cancer, but on the contrary changes the ratio in a beneficial direction. The metabolite profile is protective rather than harmful. Because these metabolites compete for the same estrogenic receptor, the increased amount of the “good metabolite” (2OHE₁) decreases the possibility that the “bad metabolite” (16OHE₁) will occupy the estrogen receptor and initiate cellular events that can lead to mutations within breast epithelial cells.

A Phase II clinical trial (first efficacy study or proof-of-concept study) is under preparation. This new clinical study is designed to evaluate primarily the effects of E₂-CDS complexed with HPβCD and delivered by the buccal route (Estredox™), administered once daily (QD) at three dose levels (0.5 mg/day, 1.0 mg/day, and 2.0 mg/day), compared to placebo, during a 12-week treatment phase, on the number and severity of hot flashes as measured by the “hot flash daily weighted severity score” (DWSS) in patients suffering from moderate to severe postmenopausal vasomotor symptoms. Secondary parameters to be evaluated are the placebo-controlled treatment effects on scores calculated from a Menopause Rating Scale (MRS) questionnaire in this patient population. Treatment compliance, and acceptability of the buccal formulation tablet will also be evaluated among the secondary parameters of the study. Disintegration time of the buccal tablets will be recorded on Day 1, 28, and 26. Safety indices before and after treatment will be evaluated too, and include physical examination with vital signs, routine safety laboratory tests, including hemostasis parameters, observed or reported adverse events, hormone levels as biomarkers of central estradiol effects, such as serum FSH, LH, prolactin, SHBG, E₂, E₁, urinary E₁ and the ratio of urinary 2OHE₁ and 16OHE₁, endometrial thickness evaluated by TVS, Pap smear, vaginal cytology (maturation index) and pH, endometrial aspirate with Pipelle, and breast examination.

The primary objective of this study is the evaluation of the effect of QD Estredox™ buccal tablet at doses of 0.5, 1.0, and 2.0 mg E₂-CDS/day compared to placebo on the number and severity of hot flashes in ambulatory postmenopausal women suffering from moderate to severe vasomotor symptoms (hot flashes) during 12 weeks of treatment.

Secondary objectives include the evaluation of placebo-controlled effects of three doses of Estredox™ (0.5, 1.0, and 2.0 mg E₂-CDS/day) on the scores of the MRS questionnaire obtained before, during (at weeks 4 and 8) and after 12 weeks of treatment. Treatment compliance and the acceptability of the buccal tablet are also to be determined and tablet disintegration times are to be recorded on three occasions (Day 1, 28, and 56).

The safety of Estredox™ treatment is to be determined by measuring vital signs, routine laboratory, including hemostasis parameters, and biomarkers to confirm central estrogenic effects, such as serum FSH, LH, E₂ together with prolactin, SHBG, and E₁, urinary E₁ and the ratio of urinary 2OHE₁/16OHE₁ before, during (except prolactin, SHBG, and urine—at weeks 4 and 8) and after the 12 weeks treatment period. Patients are to also undergo detailed gynecological examinations including endometrial thickness by TVS, Pap smear, vaginal cytology (maturation index) and pH, endometrial aspirate with Pipelle, and breast examination (mammography and ultrasound) twice; i.e. before and after treatment (week 0 and 12).

This is to be a phase II multi-center, repeated administration, double-blind, placebo-controlled dose-range study involving 80 ambulatory postmenopausal female patients randomly assigned in equal numbers into one of four treatment groups. Patients with an intact uterus, who are not under current estrogen, or estrogen-progestogen (ET/EPT), phytoestrogen, or selective estrogen receptor modulator (SERM) therapy can be enrolled. If they were under previous ET/EPT, phytoestrogen or SERM therapy, then an appropriate wash-out period will precede the enrollment of potential study candidates into the study. They first will enter a two-week no-treatment run-in phase, during which patients will be required to keep a diary to record the number and severity of hot flashes. Only patients experiencing more than 50 moderate to severe hot flashes per week (>7 per day on average) will be eligible for enrollment into the treatment phase of the study. Eligible patients will be allocated randomly and in equal numbers into one of the four treatment groups. Patients in all treatment groups will receive once daily (QD) in a double-blind fashion either one placebo buccal tablet, or one identical Estredox™ buccal tablet at a dose of 0.5, 1.0, or 2.0 mg E₂CDS/day on each morning of the study, for 84 days, under fasting conditions. The disintegration times of the buccal tablets will be recorded on Days 1, 28, and 56 when patients will self-administer the tablets at the site in the presence of study personnel. During the treatment period, patients will continue the recording of the number and severity of hot flashes. There will be interim assessments of the MRS questionnaire scores after 28 and 56 days of treatment (after weeks 4 and 8) and evaluation of compliance and adverse events. At these interim visits blood will also be sampled for determination of certain hemostasis parameters, and for serum hormone concentrations (E₂, E₁, FSH, LH only). The 12-week treatment period will fully be evaluated on Day 85 during the discharge visit.

Thus, transmucosal administration of E₂-CDS in accord with the present invention can provide effective treatment of female sexual dysfunction, including effective treatment of postmenopausal symptoms, at doses far lower than previously expected to be effective for treating women with E₂-CDS for postmenopausal symptoms. No specific dosages were ever previously suggested for treating other aspects of female sexual dysfunction such as sexual desire disorders or sexual pain disorders; in fact, treatment of these aspects of female sexual dysfunction has not been previously proposed and no relevant animal testing has been previously described in the E₂-CDS literature. Moreover, the E₂-CDS literature emphasizes the substantial and prolonged suppression of LH levels. However, while LH inhibition may be more important for certain uses of estrogens such as contraception, there does not appear to be a direct connection between LH suppression and treatment of sexual dysfunction. The low levels of E₂-CDS which can be effectively administered to women for the treatment of various aspects of sexual dysfunction in accord with this invention are particularly surprising; the 0.5 to 2.0 mg daily buccal dose, assuming approximately 30% bioavailability, calculates to an actual useable dose of only 0.15 to 0.6 mg per day, which divided by an average 60-70 kg weight, gives an approximate 0.0025 to 0.01 or less mg/kg dose in women. This is far less than the dose previously expected to be needed to effectively suppress LH and treat postmenopausal symptoms for an extended period. Obviously, dosage amounts will vary with the particular transmucosal route of administration selected and the bioavailability applicable to the selected route. The particular conditions to be relieved by administration in accord with the present invention include female sexual dysfunction, especially of the hypoactive sexual desire disorder type or of the sexual pain disorder type, as well as the symptoms linked to those disorders in postmenopausal women, whether the symptoms are associated with age or with other causes of estrogen deprivation (such as surgery). These include vaginal dryness/lack of lubrication and consequent pain associated with intercourse, vasomotor symptoms such as night sweats and hot flushes, insomnia, depression, nervousness, urinary incontinence, irritability and anxiety, even fear of pain of intercourse, all of which may be associated with the hypoactive sexual desire disorder. Of course, other conditions associated with the estrogen deprivation of menopause or postmenopause, such as osteoporosis and Alzheimer's disease, are also expected to be diminished by administration of the low-dose E₂-CDS formulations provided herein. And these dosages do not provide constant elevated peripheral estrogen levels comparable to pre-menopausal levels, such as produced by standard HRT therapy. Rather, E₂-CDS is believed to be effective in diminishing the symptoms indicated above in amounts which do not elevate average steady-state peripheral estradiol levels to above about 50-60 pg/mL. Indeed, an effective transmucosal dosage level may be selected in which such average peripheral estradiol levels do not exceed 40 pg/mL, or even 20 pg/mL or lower, with average peak estradiol peripheral levels not above 70-90 pg/mL or even lower. It is important to this invention to use repeated small doses rather than single large ones to produce average peripheral estradiol levels which are low enough (50-60 pg/mL, 40-50 pg/mL, 20 pg/mL or lower, steady-state) and not above an average of about 70-90 pg/mL peak to minimize estrogen exposure.

Investigation of Male Rat Sexual Behavior After Orchidectomy

Rationale

Castration causes the termination of sexual behavior in rats, but the sexual activity of castrated male rats can be reestablished by administration of estradiol. This has also been previously shown for administration of E₂-CDS to castrated male rats in Anderson et al. U.S. Pat. No. 4,863,911. At a single intravenous dose of 3 mg/kg in tests described therein, E₂-CDS was found to improve masculine sexual behavior in rats for 28 days by increasing the pursuit of the female by the male (i.e., decreasing mount and intromission latency) and by increasing initiation of copulatory behavior (increasing mounts and intromission). These data suggested that E₂-CDS is a potent, long-acting stimulant of the proceptive components of masculine sexual behavior. However, estradiol can interfere with ejaculation and the Anderson et al. patent and other publications relating to E₂-CDS do not address the issue of estradiol levels resulting from E₂-CDS administration as to the impact such levels may have on the treatment of all aspects of male sexual dysfunction, including erectile function. Moreover, it is now clear that the drug as used in males in the E₂-CDS literature produces unacceptably high estradiol levels in the serum for extended periods of time.

Circulating luteinizing hormone (LH) is a biomarker reflecting the CNS effects of estradiol. Estrogen diminishes the secretion of luteinizing hormone-releasing hormone (LHRH) and hence reduces the secretion of LH. Therefore, LH and estradiol levels were investigated to measure the central and peripheral effects of E₂-CDS, respectively.

Experimental Design

Adult male Sprague Dawley rats (300-400 g) from Charles River Hungary Ltd., Godollo, Hungary, were used. Animals were kept in community cages (4 animals/cage) in a climate-controlled room (23∀2EC), with a 14 hour light, 10 hour dark cycle of artificial lighting, using reversed light/dark cycle. Female rats weighing 200-250 g were brought to receptivity by subcutaneous injection of estradiol (50 pg/animal) 48 hours before testing and progesterone (0.5 mg/animal) 4 hours prior to the experiments. These hormones were dissolved in sunflower oil.

After establishment of basal behaviors as discussed below, selected animals were orchidectomized via a single midventral incision and were rehoused.

After repeated testing of animals recovered from orchidectomy as discussed below, rats were divided into four groups and treated intravenously, via a single tail vein injection, with one of the following: group 1: control (27% hydroxypropyl-β-cyclodextrin); group 2: 0.03 mg/kg E₂-CDS; group 3: 0.3 mg/kg E₂-CDS; and group 4: 3 mg/kg E₂-CDS.

Mating was observed during the dark cycle in a plexi observation cage in a room where only a dim red light was on. The male was placed in the observation cage 5 minutes prior to the female.

The following parameters were then measured:
Mount latency (ML): the time from the introduction of the female to the initial mount or intromission;
Intromission latency (IL): the time from introduction of the female to the first intromission; and
Ejaculatory latency (EL): the time from the first intromission to ejaculation.

Sessions were considered negative if IL exceeded 15 minutes. EL was only measured to check the result of castration, so as to select only those animals that showed an ejaculation latency greater than 15 minutes.

To establish basal behavior, each male was tested every 5 days until four successive and consistent behavioral patterns were achieved. This pretesting lasted for about four weeks. Approximately half of the animals tested were deemed suitable for orchidectomy.

Twenty-eight days after healing from orchidectomy, the animals were tested again (Day 0) and divided into 4 experimental groups. Only animals displaying ejaculation latencies greater than 15 minutes were included in the study.

Tests of male sexual behavior were conducted 3, 7, 14, 21, 28, 35 and 42 days after drug administration or until the effect disappeared, i.e. until no statistically significant difference was found between groups during two consecutive tests.

Behavioral patterns and related times were recorded manually by skilled observers.

After each testing day, a blood sample was taken from each animal from the retroorbital sinus under light ether anesthesia to determine serum LH and estradiol levels using double antibody and I¹²⁵ isotope-RIA kits, respectively.

Estradiol benzoate and progesterone were obtained from Richter Pharmaceuticals, Ltd., Budapest, Hungary and from Sigma Chemical Co. Inc., Budapest, Hungary, respectively. 2-Hydroxypropyl-β-cyclodextrin was purchased from Cerestar Inc., Hammond, Ind., US. E₂-CDS as a 3% complex with HPβCD (E₂-CDS-CD) was dissolved in distilled water and diluted with 27% HPβCD solution. E₂-CDS-CD was synthesized by Alchem Laboratories Corporation, Alachua, Fla., US, using the procedure of Example 2 above.

Behavioral Testing

Four weeks following orchidectomy, groups of rats were treated with one of the following drug doses via tail vein injection: E₂-CDS 0.03, 0.3, and 3 mg/kg. Blood samples were collected by orbital sinus puncture under light ether anesthesia. The samples were stored at 4EC for one hour and centrifuged at 1,000 g for 10 minutes. Plasma was separated and stored at −80EC until assayed. Plasma LH concentrations from individual samples were measured by double antibody radioimmunoassay kits obtained from Amersham Pharmacia Biotech, Rome, Italy. Plasma estradiol levels were determined by I¹²⁵ isotope radioimmunoassay kits obtained from BioChem ImmunoSystems. Concentrations of LH and estradiol were calculated by a computerized standard curve program using Prism software (Version 3.0, GraphPad, San Diego, Calif., USA). The limit of detection was 15 pg/mL.

Behavioral changes were analyzed using the Mann-Whitney U test (Siegel, Nonparametric Statistics for the Behavioral Sciences, New York; McGraw-Hill Book Company, Inc., 1956). The Fisher exact test was used for percentage comparisons (Zar, Biostatistical Analysis, Prentice Hall, Inc., Englewood Cliffs, N.J., 1974). Serum LH data were analyzed for each time and treatment group by analysis of variance (ANOVA) followed by Bonferroni posthoc test. Plasma LH and estradiol concentrations were evaluated by the computerized standard curve program of Prism software (Version 3.0, Graph Pad, San Diego, Calif., US).

Results

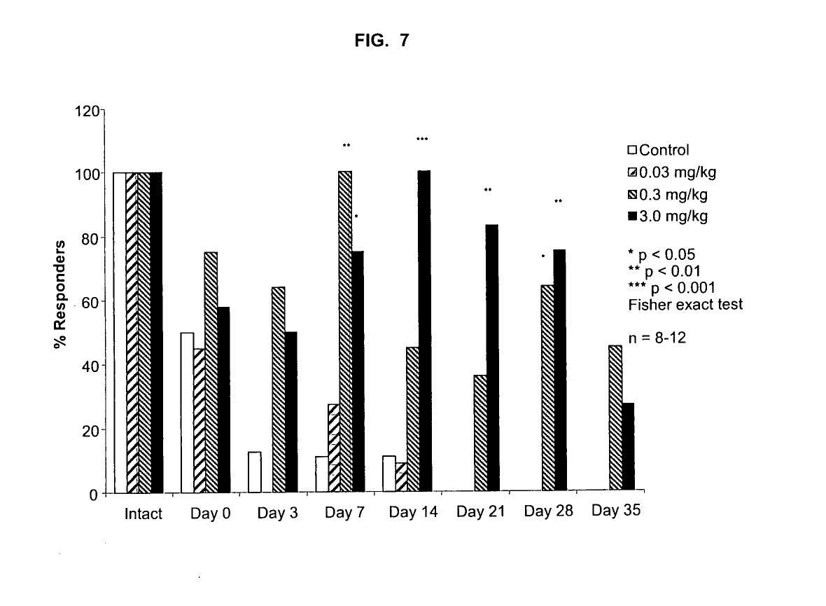

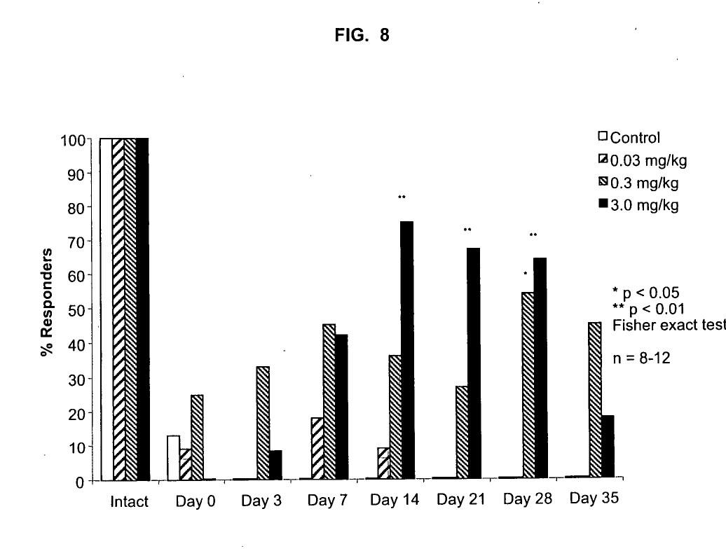

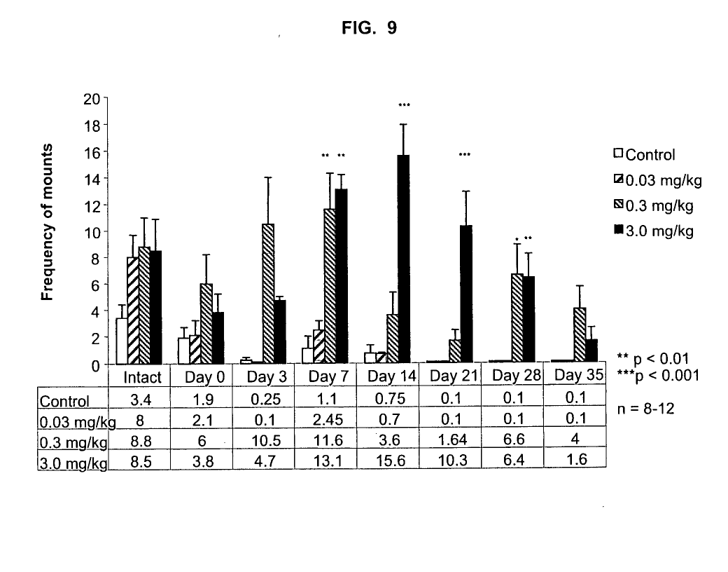

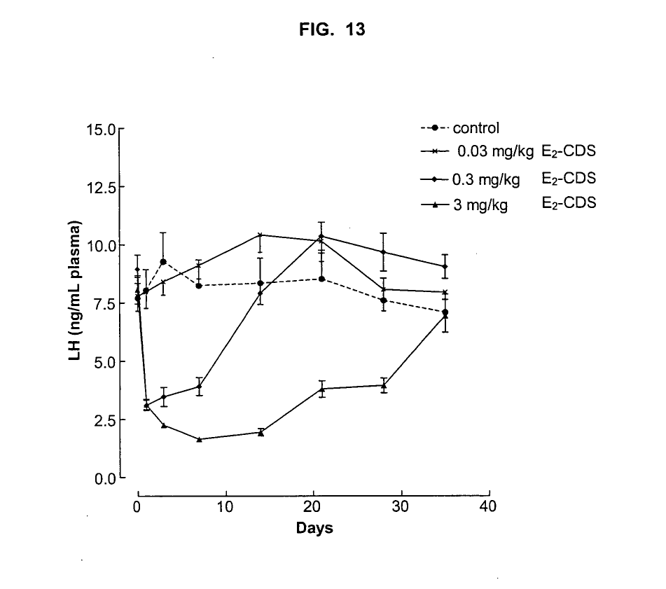

FIGS. 7-14 show the results obtained. In FIGS. 7-12, data are mean ∀SE for 8-12 animals per group; *p<0.05, **p<0.01, ***p<0.001 using the Fisher exact test or the Mann-Whitney U test, as appropriate (Fisher exact test in FIGS. 7 and 8, Mann-Whitney U tests in FIGS. 9-12). In FIG. 13, each point represents the mean ∀SEM of samples obtained from 8 to 13 rats.

Orchidectomy was found to be less effective in reducing mounting response (FIG. 7) than in reducing intromission response (FIG. 8). E₂-CDS restored mounting performance in 100% of the animals by day 7 at the dose of 0.3 mg/kg and by day 14 and day 21 at the dose of 3.0 mg/kg. The intromission performance was improved in a statistically significant manner from day 14 through day 28 at the dose of 3.0 mg/kg.

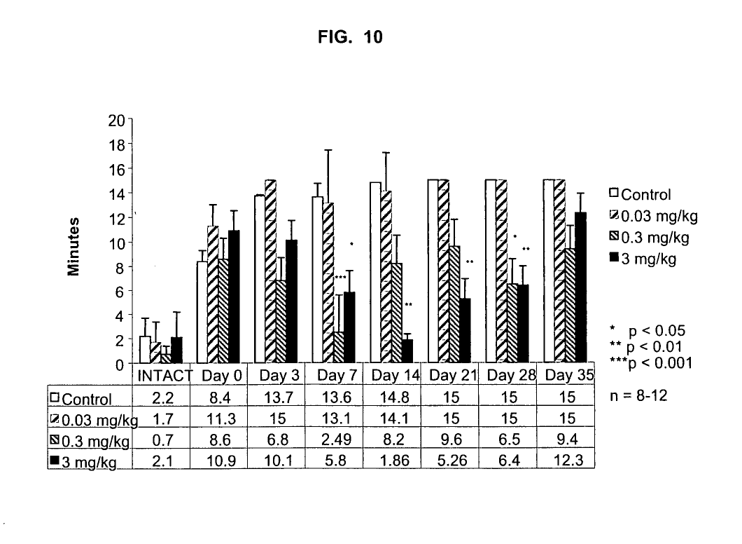

Mount frequency was significantly increased on day 7 at doses of 0.3 and 3.0 mg/kg and on days 14, 21 and 28 at the dose of 3.0 mg/kg (FIG. 9). Mount latency was sharply reduced from day 7 through day 28 for the doses of 0.3 and 3.0 mg/kg (FIG. 10).

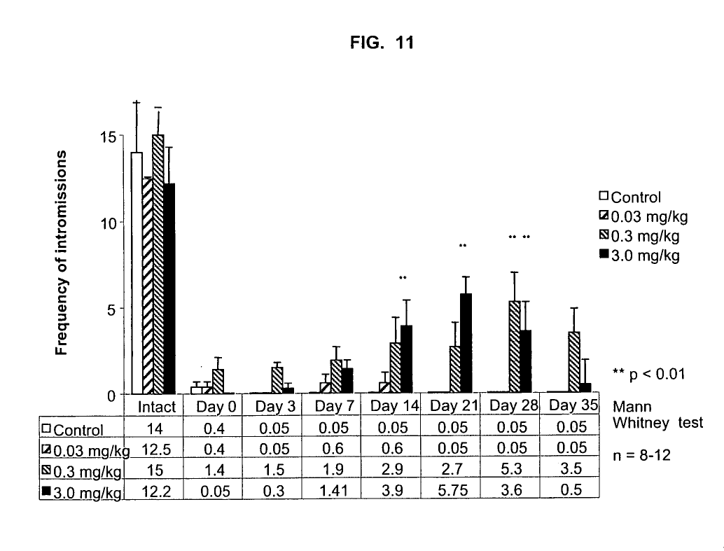

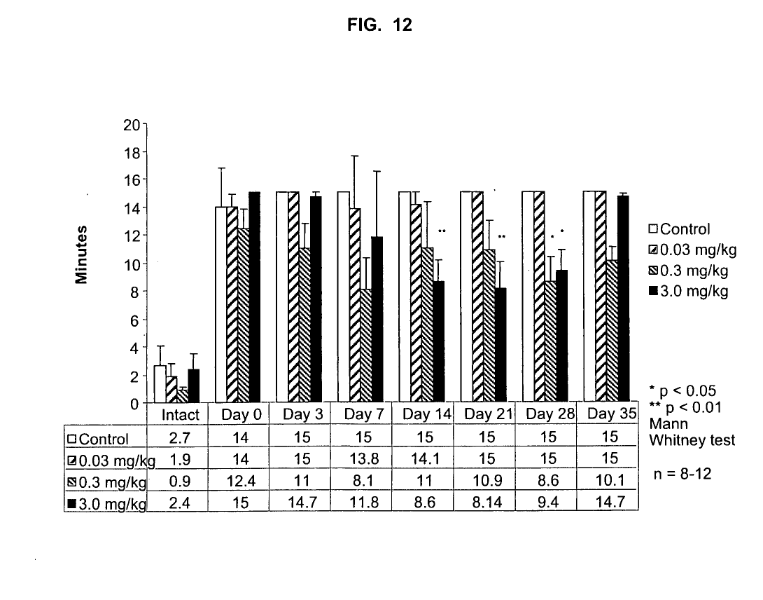

A statistically significant increase in intromission frequency and a decrease in intromission latency were observed on days 14, 21 and 28 at the dose of 3.0 mg/kg (FIGS. 11 and 12).

Thus, the effect of E₂-CDS on the re-establishment of the tested indications of copulatory behavior in male rats was significant at doses of 0.3 and 3.0 mg/kg through day 28. The dose of 0.03 mg/kg had no statistically significant effect.

Concentrations of plasma LH in intact rats were 1.1∀0.15 ng/mL. Four weeks after bilateral orchidectomy, LH levels increased to 8.13 ng/mL. At the lowest dose of E₂-CDS tested (0.03 mg/kg i.v.), plasma LH levels were not reduced. At the dose of 0.3 mg/kg i.v., the title compound significantly reduced the LH levels on days 1, 3, and 7. By day 15, there was no significant difference in the LH levels between control and treated animals. At the highest dose of E₂-CDS tested (3 mg/kg i.v.), LH levels were suppressed significantly throughout 28 days (FIG. 13).

Estradiol levels were below the limit of detection in animals treated with E₂-CDS at doses of 0.03 and 0.3 mg/kg i.v. At the highest dose tested (3 mg/kg i.v.), the estradiol level was 258∀19 pg/mL on day 1 after treatment. At this dose, the hormone level decreased by 39% to 165∀14 pg/mL on day 3 and to 61∀7.7 pg/mL on day 7. When next tested on day 14, the estradiol level for the highest dose tested was below the limit of detection. See Table 1 below. This confirms that the dosage level of E₂-CDS used in the Anderson et al. patent (3 mg/kg single i.v. dose in rats) would have produced unacceptably high peripheral estradiol levels for a prolonged period and agrees with data set forth in the Anderson et al. patent and in the E₂-CDS literature. This level is expected to be high enough to interfere with ejaculation.

TABLE 1
Plasma estradiol concentrations following E₂-CDS
or vehicle treatment in orchidectomized rats.

| | Days relative to | Plasma estradiol |
|---|---|---|
| Treatment | treatments | (pg/mL) |
| Vehicle | 0 | ND** (9/9)* |
| | 1 | ND** (9/9)* |
| | 3 | ND** (9/9)* |
| | 7 | ND** (9/9)* |
| | 14 | ND** (9/9)* |
| | 21 | ND** (9/9)* |
| | 28 | ND** (9/9)* |
| | 35 | ND** (9/9)* |
| 0.03 mg/kg E₂-CDS | 0 | 27.3 (9/11)* |
| | 1 | ND** (10/10)* |
| | 3 | ND** (10/10)* |
| | 7 | ND** (10/10)* |
| | 14 | ND** (10/10)* |
| | 21 | ND** (10/10)* |
| | 28 | ND** (10/10)* |
| | 35 | ND** (10/10)* |
| 0.3 mg/kg E₂-CDS | 0 | 31.4 (10/12)* |
| | 1 | ND** (11/11)* |
| | 3 | ND** (11/11)* |
| | 7 | ND** (11/11)* |
| | 14 | ND** (11/11)* |
| | 21 | ND** (11/11)* |
| | 28 | ND** (11/11)* |
| | 35 | ND** (11/11)* |
| 3 mg/kg E₂-CDS | 0 | 54∀21 (8/13)* |
| | 1 | 258∀19 (0/12)* |
| | 3 | 165∀14 (0/12)* |
| | 7 | 61∀7.7 (0/12) |
| | 14 | ND** (12/12)* |
| | 21 | ND** (12/12)* |
| | 28 | ND** (12/12)* |
| | 35 | ND** (12/12)* |

*Mean ∀ SEM; in parentheses: number of samples with estradiol levels below the detection limit (15 pg/mL) of the assay/number of samples per group.
**ND not detectable

The testing described above was repeated using a dose of 0.03 mg/kg administered as a single i.v. injection, and a dose of 0.01 mg/kg with daily i.v. administration for 10 days. E₂-CDS stock solution (40%) was diluted in 27% HPβCD solution.

The copulatory behavior of E₂-CDS treated groups was compared to that of the HPβCD control group at 1, 3, 7, 14, and 21 days after i.v. drug administration in the 0.03 mg/kg group and at 1, 3, 7, 14 and 21 days after initial i.v. drug administration in the 0.01 mg/kg×10 days group.

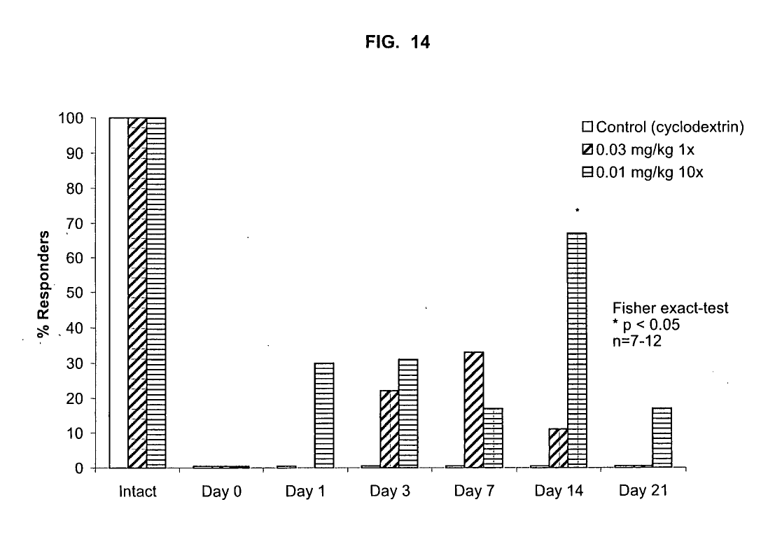

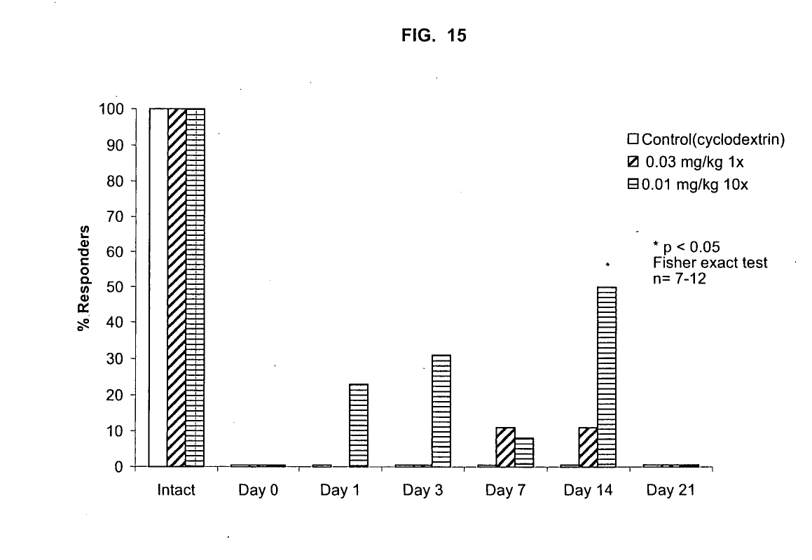

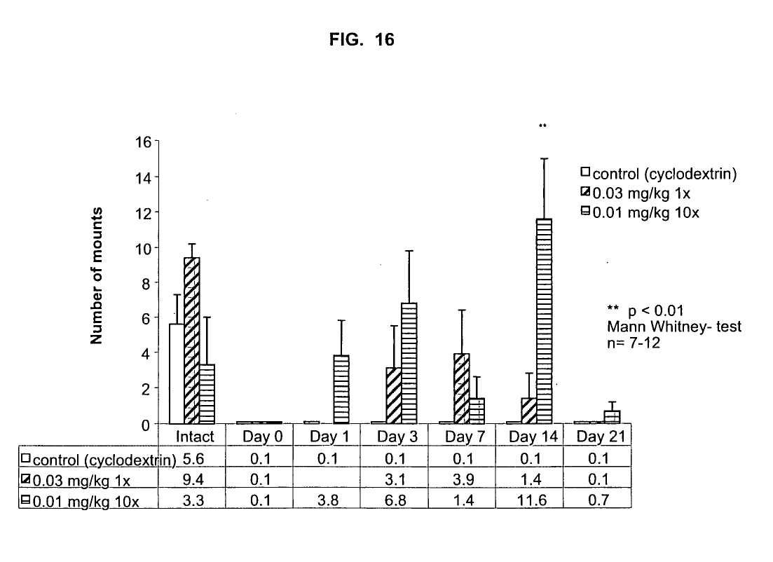

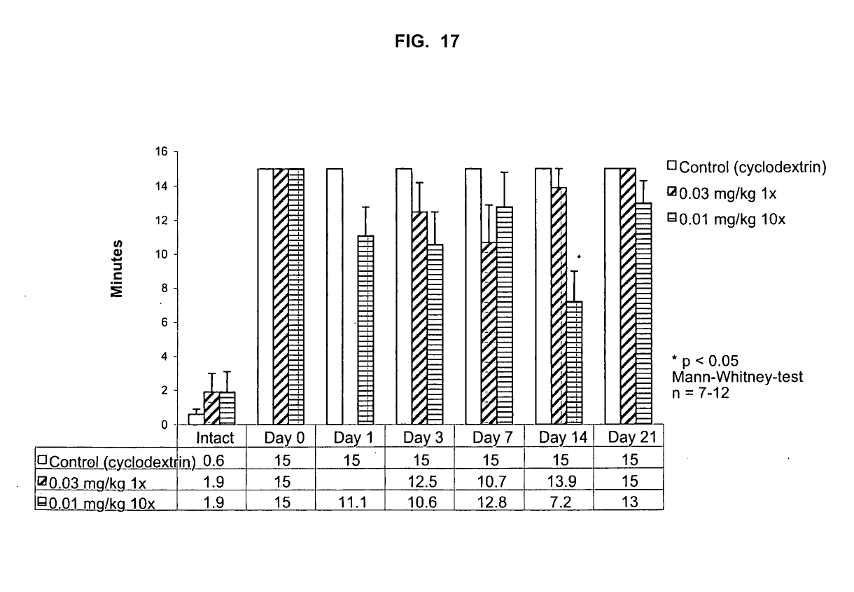

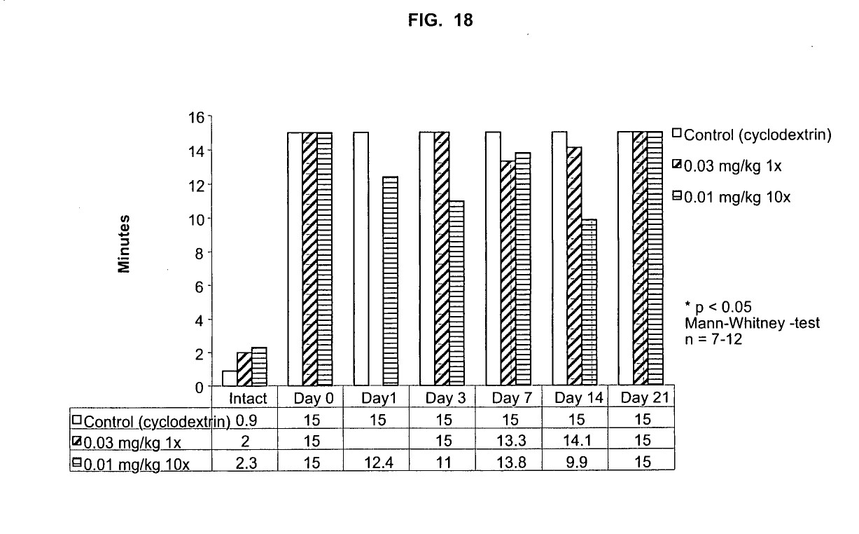

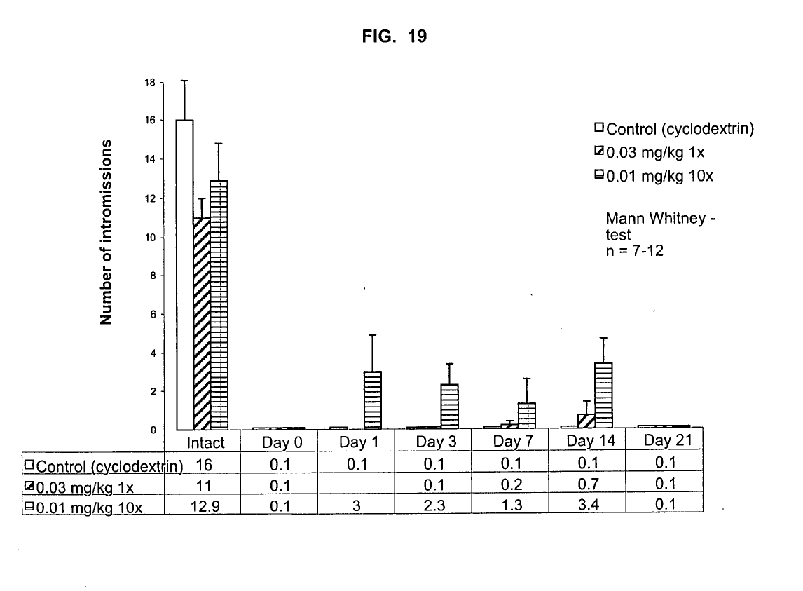

The dose of 0.01 mg/kg administered for 10 days produced significant effect by day 14. It restored mounting performance in 67% and intromission performance in 50% of animals compared to the control group (FIGS. 14 and 15). Mount frequency was increased significantly (FIG. 16). Both mount latency and intromission latency were reduced significantly (FIGS. 17 and 18). Intromission frequency was not increased significantly (FIG. 19). One animal, which had good performance before, died on day 7 under the ether anesthesia.

The single dose of 0.03 mg/kg improved sexual activity, but it was not statistically significant in any observations.

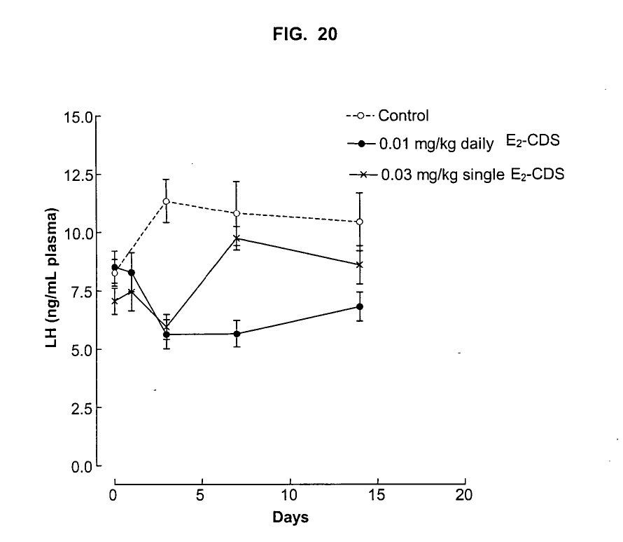

Plasma LH levels were also determined. In the repeated examination, the plasma LH level was significantly reduced at the dose of 0.01 mg/kg (10 daily injections) from day 3 to day 14. At the dose of 0.03 mg/kg (single injection), the plasma LH level was significantly reduced on day 3 only. The results of the repeated examination can be seen in FIG. 20.

At the end of the repeated examination, animals were over-anesthetized, and the prostate and seminal vesicles were removed and their weights were measured. The relative prostate and seminal vesicle weights are summarized in Table 2 below.

TABLE 2
Relative prostate and seminal vesicle weight of castrated
male rats treated with E₂-CDS

| | | Relative seminal |
|---|---|---|
| | Relative^{a} prostate weight | vesicle weight |
| Treatment | Mean ∀ SE | Mean ∀ SE |
| HPβCD i.v. | 22.15 ∀ 2.1 | 25.46 ∀ 1.55 |
| (control) | | |
| E₂-CDS | 24.78 ∀ 1.6 | 20.83 ∀ 0.61** |
| 0.01 mg/kg 10 × i.v. | | |
| E₂-CDS | 14.48 ∀ 2.1* | 19.18 ∀ 0.82** |
| 0.03 mg/kg 1 × i.v. | | |

^{a}mg/100 g body weight; n = 7-12
*p < 0.05,
**p < 0.01 Student's t test compared to control

Estradiol levels were below the limit of detection in all animals treated with E₂-CDS at doses of 0.03 mg/kg (single dose) and 0.01 mg/kg (daily for 10 days) i.v. See Table 3 below.

TABLE 3
Plasma estradiol concentrations following E₂-CDS
or vehicle treatment in orchidectomized rats.

| | Days relative to | Plasma estradiol |
|---|---|---|
| Treatment | treatments | (pg/mL) |
| Vehicle | 0 | ND** (7/7)* |
| | 1 | ND** (7/7)* |
| | 3 | ND** (7/7)* |
| | 7 | ND** (7/7)* |
| | 14 | ND** (7/7)* |
| | 21 | ND** (7/7)* |
| 0.01 mg/kg E₂-CDS | 0 | ND** (12/12)* |
| daily for 10 days i.v. | 1 | ND** (12/12)* |
| | 3 | ND** (12/12)* |
| | 7 | ND** (12/12)* |
| | 14 | ND** (12/12)* |
| | 21 | ND** (12/12)* |
| 0.03 mg/kg E₂-CDS | 0 | ND** (9/9)* |
| single dose i.v. | 1 | ND** (9/9)* |
| | 3 | ND** (9/9)* |
| | 7 | ND** (9/9)* |
| | 14 | ND** (9/9)* |
| | 21 | ND** (9/9)* |

*Mean ∀ SEM; in parentheses: number of samples with estradiol levels below the detection limit (15 pg/mL) of the assay/number of samples per group.
**ND not detectable.

The foregoing studies show that E₂-CDS can restore male sexual function in rats and indicate that symptoms of male sexual dysfunction in males, including men, can be alleviated through its administration at doses far lower than previously thought possible, while maintaining appropiate peripheral levels of estrogen. Clinical studies in women substantiate that low dose buccal administration of E₂-CDS can be correlated with animal test data and allow calculation of suitable buccal dosages for men based on the animal test data in male rats.

Administration of E₂-CDS in accord with the present invention provides effective treatment of male sexual dysfunction, at transmucosal doses far lower than previously expected to be effective for treating men with E₂-CDS for male sexual dysfunction by using repeated small doses of the compound rather than the single dose once-a-month therapy suggested earlier, to minimize or obviate elevation of peripheral estradiol levels. It also is not necessary to use a dosage high enough to significantly reduce serum LH in order to effectively treat male sexual dysfunction. The low levels of E₂-CDS which can be effectively administered to men for these purposes are particularly surprising; for example, a dose comparable to 0.01 to 0.001 mg/kg i.v. in the male rat, or a 0.01 to 0.5 mg daily buccal dose in men, is contemplated; assuming approximately 30% bioavailability, this buccal dose calculates to an actual useable dose of only 0.003 to 0.015 mg per day, which divided by an average 70-80 kg weight, gives an approximate 0.0000375 to 0.00021 or less mg/kg dose in men. Treatment is continued once-a-day or once every other day for such period of time as required until symptoms diminish, generally about 2 to 7 days in men, and treatment is resumed when symptoms recur. Obviously, dosage amounts will vary with the route of administration and the bioavailability applicable to the selected route. In any event, the method of administering E₂-CDS in accord with the present invention will utilize dosage amounts and dosage frequencies which will not substantially elevate average peripheral estradiol levels to above average normal levels in the male, i.e., will not elevate average peripheral estradiol levels more than about 10-15% above normal levels. This in turn will prevent peripheral estradiol levels from inhibiting ejaculation, so that both proceptive and consummatory aspects of male sexual behavior will be improved.

Investigation of Suppression of Stress-induced Release of ACTH and Corticosterone

Rationale

A study was undertaken to assess the anti-inflammatory effects of dexamethasone (DEX) and a brain-enhanced dexamethasone redox delivery system, referred to hereinabove as DEX-CDS. Ability to suppress stress-induced ACTH and corticosterone is a measure of the anti-inflammatory action of a test substance.

Materials and Methods

Adult male Sprague-Dawley rats (Charles River Breeding Laboratories, Wilmington, Mass.) weighing 300-325 grams were selected for use in this study. The rats were housed individually in wire-bottomed cages in a climate-controlled room (23° C.) with a 12 hour light/12 hour dark cycle of artificial lighting. The animals were maintained on this light cycle for 10 days prior to the start of the experiment. Purina cat chow and tap water were provided ad libitum.

A single tail vein injection of (1) DEX-CDS at 10 mg/kg body weight (2) DEX at the equimolar dose of 7.65 mg/kg body weight or (3) the drug vehicle, 2-hydroxypropyl-β-cyclodextrin (HPβCD), at a volume of 5 mL/kg, was administered or each rat. Rats then were subjected to either no stress or light restraint stress for 5 or 15 minutes on days 0, 1, 3, 5 or 7. Each experimental group consisted of six animals. For light restraint testing, the rats were placed in individual wire-mesh cages which permitted limited movement in a lateral or longitudinal direction. Nonstressed control rats were gently removed from their cages and immediately sacrificed. All animals were killed immediately at the end of the stress periods by decapitation. Trunk blood was rapidly collected in iced tubes containing EDTA, and the plasma was separated by cold centrifugation and stored at −80° C. until radioimmunoassay.

Plasma ACTH was measured in duplicate by radioimmunoassay using a double-antibody technique (Diagnostic Products Corp., Los Angeles, Calif.). The intra-assay coefficient of variation was 8.25%; and the limit of sensitivity for the assay was 17 pg/mL of plasma.

Plasma corticosterone was measured in duplicate by radioimmunoassay using a double-antibody technique (Cambridge Medical Tech., Inc., Billerica, Mass.). The intra-assay coefficient of variation was <2.63% and the interassay coefficient of variation was 7.1%. The limit of sensitivity for the assay was 0.39 ng/mL of plasma.

The significance of differences among mean values was determined by analysis of variance (Anova) and Student-Newman-Keuls tests. The level of probability for all tests was 0.05.

Results

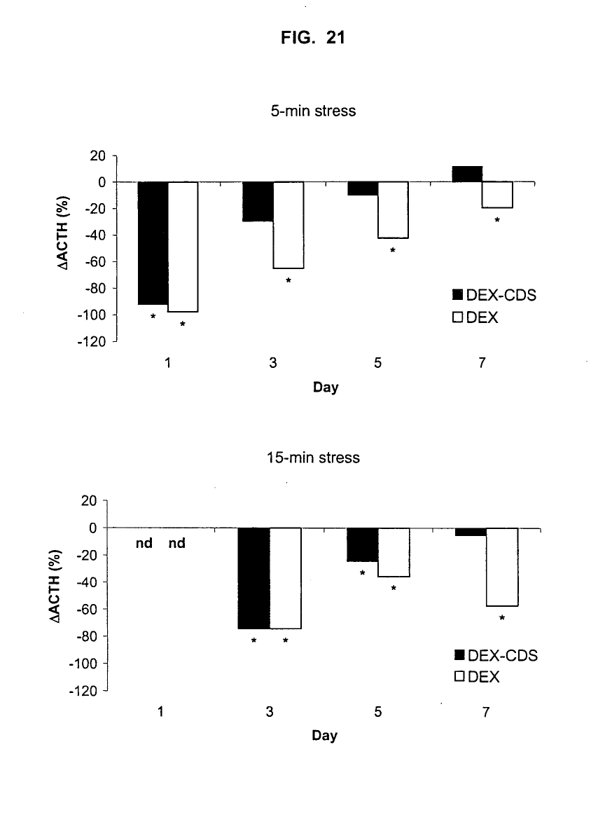

Baseline ACTH levels, obtained in rats which received the drug vehicle only, were 24.7±1.2 pg/mL; the 5-minute restraint stress period elevated these ACTH levels to 94.2±5.7 pg/mL. As shown in the upper portion of FIG. 21, a single intravenous injection of either DEX-CDS or DEX blocked stress-related elevations in ACTH on days 1 and 3. In animals treated with DEX, the ACTH response to the 5-minute stress period returned to control levels by day 3. In rats treated with DEX-CDS, suppression of ACTH elevations continued through day 5.

In the 15-minute stress test, vehicle only rats exhibited ACTH levels of 167.3±17.8 pg/mL when stressed for 15 minutes, while non-stress ACTH levels (26.0±1.8 pg/mL) did not differ from those observed in the 5-minute study. As shown in the lower portion of FIG. 21, both DEX and DEX-CDS blocked stress-related rises in ACTH at day 3; on the other hand, as shown, DEX did not suppress stress-related rises in ACTH on days 5 or 7, while DEX-CDS suppressed elevations in ACTH for at least 7 days.

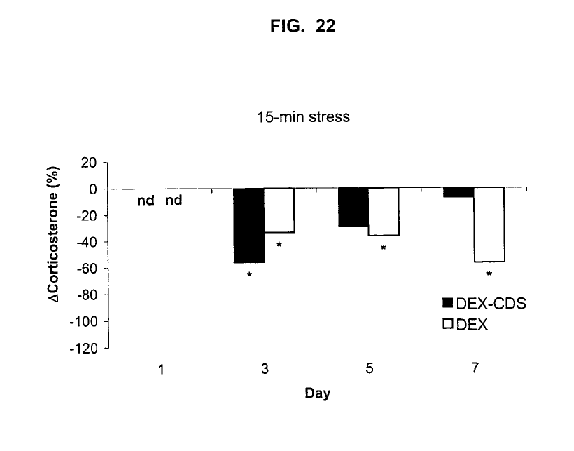

Serum corticosterone levels were measured in the 15-minute stress study and the results are depicted in FIG. 22. Corticosterone levels were elevated after 15 minutes of restraint stress from 33.3±1.4 ng/mL to 63.1±3.1 ng/mL, an 89% increase. DEX suppressed corticosterone levels by 55% on day 3, but was not effective in significantly suppressing the corticosterone response after that. In contrast, DEX-CDS significantly suppressed corticosterone response to stress on days 3, 5 and 7 by 33%, 37% and 56%, respectively.

These results indicate that a single dose of DEX-CDS can suppress stress-induced elevations in ACTH and concomitant rise in corticosterone for a longer period than dexamethasone itself. Other tests have shown higher brain levels and lower peripheral levels of dexamethasone after treatment with DEX-CDS than after treatment with dexamethasone itself. Like E₂-CDS, DEX-CDS has been found to be a long-acting steroid in intravenous studies in rats. Therefore, it is believed that the clinical studies of E₂-CDS in women suggest that appropriate dosages for transmucosal administration of DEX-CDS can be extrapolated from the intravenous rat stress studies for treatment of cerebral edema, using low doses such as from about 5 to about 20 mg administered more frequently than in the rat studies (daily or more often), while minimizing typical side-effects such as adrenal atrophy, an Addison's disease-like syndrome and anti-insulin effects on the liver, liver degeneration and necrosis.

As will be apparent to those skilled in the art to which the invention pertains, the present invention may be embodied in forms other than those specifically disclosed above without departing from the spirit or essential characteristics of he invention. The particular embodiments of the invention described above are, therefore, to be considered as illustrative and not restrictive. The scope of the invention is as set forth in the appended claims rather than being limited to the foregoing description.

1. A pharmaceutical composition comprising an essentially saturated S-CDS-cyclodextrin complex formulated into a transmucosal dosage form, wherein S-CDS is a compound of the formula:
DDHC]ₙ (I)
or a non-toxic pharmaceutically acceptable salt thereof, wherein:
(a) D is the residue of a steroidal female sex hormone having one or two reactive hydroxyl functional groups, one such hydroxyl group being a 17β-hydroxy substituent, said residue having a hydrogen atom absent from at least one of the reactive hydroxyl functional groups; n is a positive integer equal to the number of said functional groups from which a hydrogen atom is absent; and [DHC] is a radical of the formula 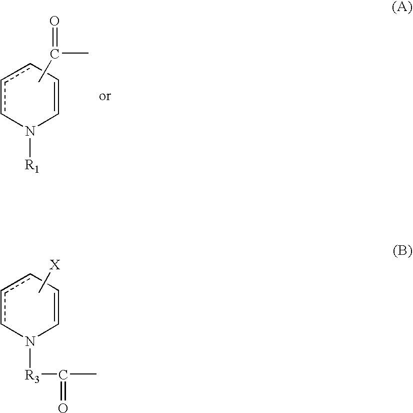
wherein the dotted line indicates the presence of a double bond in either the 4- or 5-position of the dihydropyridine ring; R₁ is C₁-C₇ alkyl or C₇-C₁₀ aralkyl; R₃ is C₁-C₃ alkylene; X is —CONR′R″ wherein each of R′ and R″, which are the same or different, is H or C₁-C₇ alkyl, or X is —COOR′″ wherein R′″ is C₁-C₇ alkyl or C₇-C₁₀ aralkyl; the carbonyl grouping in (A) is attached at the 2-, 3- or 4-position of the dihydropyridine ring; and the X grouping in (B) is attached at the 2-, 3- or 4-position of the dihydropyridine ring;
(b) D is the residue of an anti-inflammatory steroid having at least one reactive hydroxyl functional group, one such hydroxyl group being a 21-hydroxy substituent, said residue being characterized by the absence of a hydrogen atom from at least one of the reactive hydroxyl functional groups; and n and [DHC] are defined as above; or
(c) D is the residue of a steroidal androgen having a reactive 17β-hydroxyl functional group, said residue having a hydrogen atom absent from the 17β-hydroxyl functional group; n is one and [DHC] is defined as above;
said composition being substantially free of cyclodextrin in excess of the minimum amount required to maximize the amount of S-CDS in the complex, the amount of S-CDS in the complex being an amount effective to elicit a therapeutic response while maintaining acceptably low peripheral steroid levels.

2. A pharmaceutical composition comprising an essentially saturated S-CDS-cyclodextrin complex formulated into a transmucosal dosage form, wherein S-CDS is a compound of the formula:
DDHC]ₙ (I)
or a non-toxic pharmaceutically acceptable salt thereof, wherein:
(a) D is the residue of a steroidal female sex hormone having one or two reactive hydroxyl functional groups, one such hydroxyl group being a 17β-hydroxy substituent, said residue having a hydrogen atom absent from at least one of the reactive hydroxyl functional groups; n is a positive integer equal to the number of said functional groups from which a hydrogen atom is absent; and [DHC] is a radical of the formula 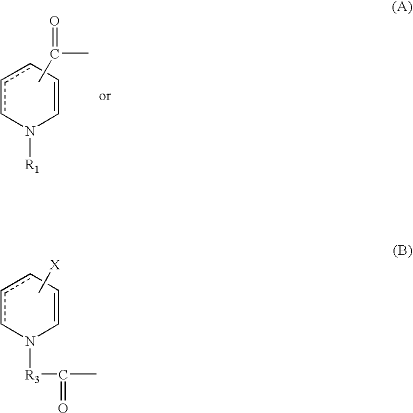
wherein the dotted line indicates the presence of a double bond in either the 4- or 5-position of the dihydropyridine ring; R₁ is C₁-C₇ alkyl or C₇-C₁₀ aralkyl; R₃ is C₁-C₃ alkylene; X is —CONR′R″ wherein each of R′ and R″, which are the same or different, is H or C₁-C₇ alkyl, or X is —COOR′″ wherein R′″ is C₁-C₇ alkyl or C₇-C₁₀ aralkyl; the carbonyl grouping in (A) is attached at the 2-, 3- or 4-position of the dihydropyridine ring; and the X grouping in (B) is attached at the 2-, 3- or 4-position of the dihydropyridine ring;
(b) D is the residue of an anti-inflammatory steroid having at least one reactive hydroxyl functional group, one such hydroxyl group being a 21-hydroxy substituent, said residue being characterized by the absence of a hydrogen atom from at least one of the reactive hydroxyl functional groups; and n and [DHC] are defined as above; or
(c) D is the residue of a steroidal androgen having a reactive 17β-hydroxyl functional group, said residue having a hydrogen atom absent from the 17β-hydroxyl functional group; n is one and [DHC] is defined as above;
said composition being substantially free of cyclodextrin in excess of the minimum amount required to maintain substantially all of the S-CDS in the complex, the amount of S-CDS in the complex being an amount effective to elicit a therapeutic response while maintaining acceptably low peripheral steroid levels.

3. The composition according to claim 1 or 2, wherein the essentially saturated S-CDS-cyclodextrin complex is formulated into a solid transmucosal dosage form.

4. The composition according to claim 3, wherein the solid transmucosal dosage form is a solid buccal dosage form.

5. A composition according to any one of the preceding claims, wherein n is one.

6. A composition according to any one of the preceding claims, wherein [DHC] has formula (A) wherein R₁ is methyl.

7. A composition according to any one of claims 1 to 5, wherein [DHC] has formula (B) wherein R₃ is —CH₂— and X is —CONH₂ or —COOR′″ wherein R′″ is methyl or ethyl.

8. A composition according to any one of the preceding claims, wherein D is the residue of a steroidal estrogen.

9. A composition according to claim 8, wherein the estrogen is estradiol, ethinyl estradiol, estrone, estradiol 3-methyl ether, estradiol benzoate or mestranol.

10. A composition according to claim 9, wherein the estrogen is estradiol.

11. A composition according to claim 10, wherein the S-CDS is selected from the group consisting of 17β-[(1-methyl-1,4-dihydro-3-pyridinyl)carbonyloxy]estra-1,3,5(10)-trien-3-ol, 17β-[(1-methyl-1,2-dihydropyridin-3-yl)carbonyloxy]estra-1,3,5(10)-trien-3-ol and 17β-[(1-methyl-1,6-dihydropyridin-3-yl)carbonyloxy]estra-1,3,5(10)-trien-3-ol.

12. A composition according to any one of claims 1 to 7, wherein D is the residue of a steroidal progestin.

13. A composition according to claim 12, wherein the progestin is norethindrone, ethisterone, norgestrel or norethynodrel.

14. A composition according to any one of claims 1 to 7, wherein D is the residue of an anti-inflammatory steroid.

15. A composition according to claim 14, wherein the anti-inflammatory steroid is dexamethasone, hydrocortisone, betamethasone, cortisone, flumethasone, fluprednisolone, meprednisone, methylprednisolone, prednisolone, prednisone, triamcinolone, cortodoxone, fludrocortisone, fluandrenolide or paramethasone.

16. A composition according to claim 15, wherein the anti-inflammatory steroid is dexamethasone.

17. A composition according to claim 16, wherein the S-CDS is selected the group consisting of 9-fluoro-11β,17-dihydroxy-16α-methyl-21-{[(1-methyl-1,4-dihydropyridin-3-yl)carbonyl]oxy}pregna-1,4-diene-3,20-dione, 9-fluoro-11β,17-dihydroxy-16α-methyl-21-{[(1-methyl-1,2-dihydropyridin-3-yl)carbonyl]oxy}pregna-1,4-diene-3,20-dione and 9-fluoro-11β,17-dihydroxy-16α-methyl-21-{[(1-methyl-1,6-dihydropyridin-3-yl)carbonyl]oxy}pregna-1,4-diene-3,20-dione.

18. A composition according to any one of claims 1 to 7, wherein D is the residue of a steroidal androgen.

19. A composition according to claim 18, wherein the steroidal androgen is testosterone or methyltestosterone.

20. A composition according to claim 19, wherein the S-CDS is 17β-{[(3″-carbomoyl-1′,4′-dihydropyridinyl)acetyl]oxy}androst-4-en-3-one or 17β-[(1,4-dihydro-1-methyl-3-pyridinylcarbonyl)oxy]androst-4-en-3-one.

21. A composition according to any one of the preceding claims, wherein the cyclodextrin is hydroxypropyl-β-cyclodextrin, hydroxypropyl-γ-cyclodextrin, hydroxyethyl-β-cyclodextrin, hydroxyethyl-γ-cyclodextrin, carboxymethyl-β-cyclodextrin, carboxymethyl-γ-cyclodextrin, carboxyethyl-β-cyclodextrin, carboxyethyl-γ-cyclodextrin, β-cyclodextrin sulfobutyl ether, carboxymethylethyl-β-cyclodextrin, carboxymethylethyl-γ-cyclodextrin, dimethyl-β-cyclodextrin or randomly methylated β-cyclodextrin.

22. A composition according to claim 21, wherein the cyclodextrin is hydroxypropyl-β-cyclodextrin, hydroxypropyl-γ-cyclodextrin, carboxymethyl-β-cyclodextrin or carboxymethyl-γ-cyclodextrin.

23. A composition according to any one of the preceding claims, wherein the formulation is anhydrous.

24. A composition according to any one of the preceding claims, wherein the approximate molar ratio of the S-CDS to cyclodextrin corresponds to a point located on a phase solubility diagram for substantially saturated complexes of the S-CDS in varying concentrations of the cyclodextrin.

25. A composition according to any one of preceding claims, formulated into a buccal tablet, buccal wafer or buccal patch.

26. A buccal tablet, buccal wafer or buccal patch comprising an anhydrous formulation of a substantially saturated complex of the compound 17β-[(1-methyl-1,4-dihydro-3-pyridinyl)carbonyloxy]estra-1,3,5(10)-trien-3-ol with a hydroxyalkyl, carboxyalkyl or carboxymethylethyl derivative of β- or γ-cyclodextrin comprising from about 0.01 to 2.0 mg of said compound.

27. A buccal tablet, buccal wafer or buccal patch according to claim 26, wherein the cyclodextrin is hydroxypropyl-β-cyclodextrin, hydroxypropyl-γ-cyclodextrin, hydroxyethyl-β-cyclodextrin, hydroxyethyl-γ-cyclodextrin, carboxymethyl-β-cyclodextrin, carboxymethyl-γ-cyclodextrin, carboxyethyl-β-cyclodextrin, carboxyethyl-γ-cyclodextrin or carboxymethylethyl-β-cyclodextrin.

28. A buccal tablet, buccal wafer or buccal patch comprising an anhydrous formulation of a substantially saturated complex of the compound 17β-[(1-methyl-1,4-dihydro-3-pyridinyl)carbonyloxy]estra-1,3,5(10)-trien-3-ol with a hydroxyalkyl or carboxyalkyl derivative of β- or γ-cyclodextrin comprising from about 0.01 up to but not including 0.5 mg of said compound.

29. A buccal tablet, buccal wafer or buccal patch comprising an anhydrous formulation of a substantially saturated complex of the compound 17β-[(1-methyl-1,4-dihydro-3-pyridinyl)carbonyloxy]estra-1,3,5(10)-trien-3-ol with a hydroxyalkyl or carboxyalkyl derivative of β- or γ-cyclodextrin comprising from about 0.5 to about 2.0 mg of said compound.

30. A tablet, wafer or patch according to claim 28 or 29, wherein the cyclodextrin is hydroxypropyl-β-cyclodextrin, hydroxypropyl-γ-cyclodextrin, hydroxyethyl-β-cyclodextrin or hydroxyethyl-γ-cyclodextrin.

31. A tablet, wafer or patch according to claim 30, wherein the cyclodextrin is 2-hydroxypropyl-β-cyclodextrin or 2-hydroxypropyl-γ-cyclodextrin.

32. A tablet, wafer or patch according to claim 28 or 29, wherein the cyclodextrin is carboxymethyl-β-cyclodextrin, carboxymethyl-γ-cyclodextrin carboxyethyl-β-cyclodextrin or carboxyethyl-γ-cyclodextrin.

33. A tablet, wafer or patch according to claim 32, wherein the cyclodextrin is carboxymethyl-β-cyclodextrin or carboxymethyl-γ-cyclodextrin.

34. A method for enhancing the transmucosal bioavailability of an S-CDS comprising transmucosally administering to a subject in need thereof a pharmaceutical composition comprising an essentially saturated S-CDS-cyclodextrin complex formulated into a transmucosal dosage form, said composition being substantially free of cyclodextrin in excess of the minimum amount required to maximize the amount of S-CDS in the complex, said S-CDS being a compound of the formula:
DDHC]ₙ (I)
or a non-toxic pharmaceutically acceptable salt thereof, wherein:
(a) D is the residue of a steroidal female sex hormone having one or two reactive hydroxyl functional groups, one such hydroxyl group being a 17β-hydroxy substituent, said residue having a hydrogen atom absent from at least one of the reactive hydroxyl functional groups; n is a positive integer equal to the number of said functional groups from which a hydrogen atom is absent; and [DHC] is a radical of the formula 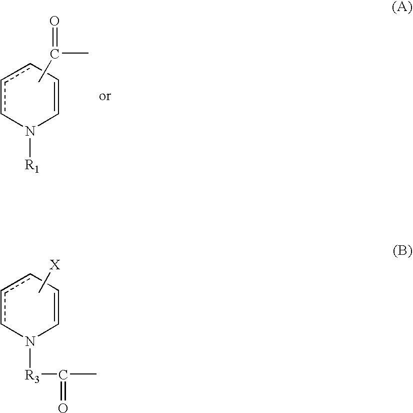
wherein the dotted line indicates the presence of a double bond in either the 4- or 5-position of the dihydropyridine ring; R₁ is C₁-C₇ alkyl or C₇-Cl₀ aralkyl; R₃ is C₁-C₃ alkylene; X is —CONR′R″ wherein each of R′ and R″, which are the same or different, is H or C₁-C₇ alkyl, or X is —COOR′″ wherein R′″ is C₁-C₇ alkyl or C₇-C₁₀ aralkyl; the carbonyl grouping in (A) is attached at the 2-, 3- or 4-position of the dihydropyridine ring; and the X grouping in (B) is attached at the 2-, 3- or 4-position of the dihydropyridine ring;
(b) D is the residue of an anti-inflammatory steroid having at least one reactive hydroxyl functional group, one such hydroxyl group being a 21-hydroxy substituent, said residue being characterized by the absence of a hydrogen atom from at least one of the reactive hydroxyl functional groups; and n and [DHC] are defined as above; or
(c) D is the residue of a steroidal androgen having a reactive 17β-hydroxyl functional group, said residue having a hydrogen atom absent from the 17β-hydroxyl functional group; n is one and [DHC] is defined as above;
the amount of S-CDS in the complex being an amount effective to elicit a therapeutic response while maintain acceptably low peripheral steroid levels.

35. A method for enhancing the transmucosal bioavailability of an S-CDS comprising transmucosally administering to a subject in need thereof a pharmaceutical composition comprising an essentially saturated S-CDS-cyclodextrin complex formulated into a transmucosal dosage form, said composition being substantially free of cyclodextrin in excess of the minimum amount required to maintain substantially all of the S-CDS in the complex, said S-CDS being a compound of the formula:
DDHC]ₙ (I)
or a non-toxic pharmaceutically acceptable salt thereof, wherein:
(a) D is the residue of a steroidal female sex hormone having one or two reactive hydroxyl functional groups, one such hydroxyl group being a 17β-hydroxy substituent, said residue having a hydrogen atom absent from at least one of the reactive hydroxyl functional groups; n is a positive integer equal to the number of said functional groups from which a hydrogen atom is absent; and [DHC] is a radical of the formula 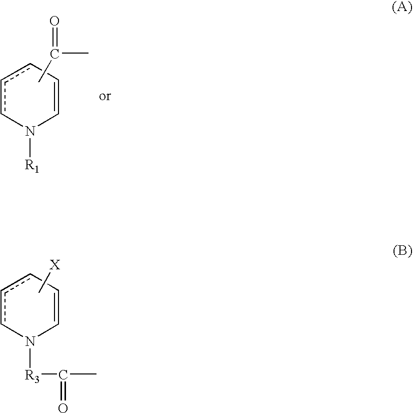
wherein the dotted line indicates the presence of a double bond in either the 4- or 5-position of the dihydropyridine ring; R₁ is C₁-C₇ alkyl or C₇-C₁₀ aralkyl; R₃ is C₁-C₃ alkylene; X is —CONR′R″ wherein each of R′ and R″, which are the same or different, is H or C₁-C₇ alkyl, or X is —COOR′″ wherein R′″ is C₁-C₇ alkyl or C₇-C₁₀ aralkyl; the carbonyl grouping in (A) is attached at the 2-, 3- or 4-position of the dihydropyridine ring; and the X grouping in (B) is attached at the 2-, 3- or 4-position of the dihydropyridine ring;
(b) D is the residue of an anti-inflammatory steroid having at least one reactive hydroxyl functional group, one such hydroxyl group being a 21-hydroxy substituent, said residue being characterized by the absence of a hydrogen atom from at least one of the reactive hydroxyl functional groups; and n and [DHC] are defined as above; or
(c) D is the residue of a steroidal androgen having a reactive 17β-hydroxyl functional group, said residue having a hydrogen atom absent from the 17β-hydroxyl functional group; n is one and [DHC] is defined as above;
the amount of S-CDS in the complex being an amount effective to elicit a therapeutic response while maintaining acceptably low peripheral steroid levels.

36. A method according to claim 34 or 35, wherein the essentially saturated S-CDS-cyclodextrin complex is formulated into a solid transmucosal dosage form.

37. A method according to claim 36, wherein the solid transmucosal dosage form is a solid buccal dosage form.

38. A method according to any one of claims 34 to 37, wherein n is one.

39. A method according to any one of claims 34 to 38, wherein [DHC] has formula (A) wherein R₁ is methyl.

40. A method according to any one of claims 34 to 38, wherein [DHC] has formula (B) wherein R₃ is —CH₂— and X is —CONH₂ or —COOR′″ wherein R′″ is methyl or ethyl.

41. A method according to any one of claims 34 to 40, wherein D is the residue of a steroidal estrogen.

42. A method according to claim 41, wherein the estrogen is estradiol.

43. A method according to 42, wherein the S-CDS is 17β-[(1-methyl-1,4-dihydro-3-pyridinyl)carbonyloxy]estra-1,3,5(10)-trien-3-ol, 17,8-[(1-methyl-1,2-dihydropyridin-3-yl)carbonyloxy]estra-1,3,5(10)-trien-3-ol and 17,f-[(1-methyl-1,6-dihydropyridin-3-yl)carbonyloxy]estra-1,3,5(10)-trien-3-ol.

44. A method according to any one of claims 34 to 40, wherein D is the residue of an anti-inflammatory steroid.

45. A method according to claim 44, wherein the anti-inflammatory steroid is dexamethasone.

46. A method according to claim 45, wherein the S-CDS in 9-fluoro-11β,17-dihydroxy-16α-methyl-21-{[(1-methyl-1,4-dihydropyridin-3-yl)carbonyl]oxy}pregna-1,4-diene-3,20-dione.

47. A method according to any one of claims 34 to 46, wherein the cyclodextrin is hydroxypropyl-β-cyclodextrin, hydroxypropyl-γ-cyclodextrin, hydroxyethyl-β-cyclodextrin, hydroxyethyl-γ-cyclodextrin, carboxymethyl-β-cyclodextrin, carboxymethyl-γ-cyclodextrin, carboxyethyl-β-cyclodextrin, carboxyethyl-γ-cyclodextrin, β-cyclodextrin sulfobutyl ether, carboxymethylethyl-β-cyclodextrin, carboxymethylethyl-γ-cyclodextrin, dimethyl-β-cyclodextrin or randomly methylated β-cyclodextrin.

48. A method according to claim 47, wherein the cyclodextrin is hydroxypropyl-β-cyclodextrin, hydroxypropyl-γ-cyclodextrin, carboxymethyl-β-cyclodextrin or carboxymethyl-γ-cyclodextrin.

49. A method according to any one of claims 34 to 48, wherein the formulation is anhydrous.

50. A method according to any one of claims 34 to 49, wherein the approximate molar ratio of the S-CDS to cyclodextrin corresponds to a point located on a phase solubility diagram for essentially saturated complexes of the S-CDS in varying concentrations of the cyclodextrin.

51. A method according to any one of claims 34 to 50, formulated into a buccal tablet, buccal wafer or buccal patch.

52. A method for enhancing the transmucosal bioavailability of the compound 17β-[( 1-methyl-1,4-dihydro-3-pyridinyl)carbonyloxy]estra-1,3,5(10)-trien-3-ol comprising buccally administering to a subject in need thereof a buccal tablet, buccal wafer or buccal patch comprising an anhydrous formulation of a substantially saturated complex of said compound with a hydroxyalkyl, carboxyalkyl or carboxymethylethyl derivative of β- or γ-cyclodextrin comprising from about 0.01 to about 2.0 mg of said compound.

53. A method according to claim 52, wherein the cyclodextrin is hydroxypropyl-β-cyclodextrin, hydroxypropyl-γ-cyclodextrin, hydroxyethyl-β-cyclodextrin, hydroxyethyl-γ-cyclodextrin, carboxymethyl-β-cyclodextrin, carboxymethyl-γ-cyclodextrin, carboxyethyl-β-cyclodextrin, carboxyethyl-γ-cyclodextrin or carboxymethylethyl-β-cyclodextrin.

54. A method according to claim 53, wherein the cyclodextrin is hydroxypropyl-β-cyclodextrin, hydroxypropyl-γ-cyclodextrin, carboxymethyl-β-cyclodextrin or carboxymethyl-γ-cyclodextrin and the amount of said compound is from about 0.5 to about 2.0 mg.

55. A method according to claim 53, wherein the cyclodextrin is hydroxypropyl-β-cyclodextrin, hydroxypropyl-γ-cyclodextrin, carboxymethyl-β-cyclodextrin or carboxymethyl-γ-cyclodextrin and the amount of said compound is from about 0.01 up to but not including 0.5 mg.

56. Use of an essentially saturated S-CDS-cyclodextrin complex in the formulation of a transmucosal dosage form substantially free of cyclodextrin in excess of the minimum amount required to maximize the amount of S-CDS in the complex, for enhancing the transmucosal bioavailability of the S-CDS, said S-CDS being a compound of the formula:
DDHC]ₙ (I)
or a non-toxic pharmaceutically acceptable salt thereof, wherein:
(a) D is the residue of a steroidal female sex hormone having one or two reactive hydroxyl functional groups, one such hydroxyl group being a 17β-hydroxy substituent, said residue having a hydrogen atom absent from at least one of the reactive hydroxyl functional groups; n is a positive integer equal to the number of said functional groups from which a hydrogen atom is absent; and [DHC] is a radical of the formula 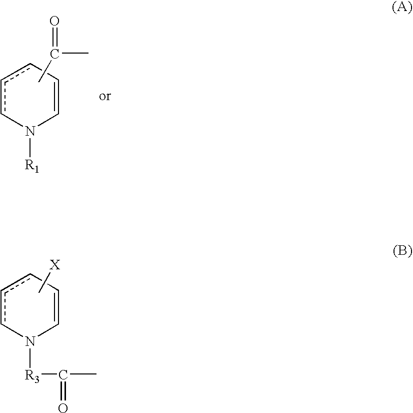
wherein the dotted line indicates the presence of a double bond in either the 4- or 5-position of the dihydropyridine ring; R₁ is C₁-C₇ alkyl or C₇-C₁₀ aralkyl; R₃ is C₁-C₃ alkylene; X is —CONR′R″ wherein each of R′ and R″, which are the same or different, is H or C₁-C₇ alkyl, or X is —COOR′″ wherein R′″ is C₁-C₇ alkyl or C₇-C₁₀ aralkyl; the carbonyl grouping in (A) is attached at the 2-, 3- or 4-position of the dihydropyridine ring; and the X grouping in (B) is attached at the 2-, 3- or 4-position of the dihydropyridine ring;
(b) D is the residue of an anti-inflammatory steroid having at least one reactive hydroxyl functional group, one such hydroxyl group being a 21-hydroxy substituent, said residue being characterized by the absence of a hydrogen atom from at least one of the reactive hydroxyl functional groups; and n and [DHC] are defined as above; or
(c) D is the residue of a steroidal androgen having a reactive 17β-hydroxyl functional group, said residue having a hydrogen atom absent from the 17β-hydroxyl functional group; n is one and [DHC] is defined as above;
the amount of S-CDS in the complex being an amount effective to elicit a therapeutic response while maintaining acceptably low peripheral steroid levels.

57. Use of an essentially saturated S-CDS-cyclodextrin complex in the formulation of a transmucosal dosage form substantially free of cyclodextrin in excess of the minimum amount required to maintain substantially all of the S-CDS in the complex, for enhancing the transmucosal bioavailability of the S-CDS, said S-CDS being a compound of the formula:
DDHC]ₙ (I)
or a non-toxic pharmaceutically acceptable salt thereof, wherein:
(a) D is the residue of a steroidal female sex hormone having one or two reactive hydroxyl functional groups, one such hydroxyl group being a 17β-hydroxy substituent, said residue having a hydrogen atom absent from at least one of the reactive hydroxyl functional groups; n is a positive integer equal to the number of said functional groups from which a hydrogen atom is absent; and [DHC] is a radical of the formula 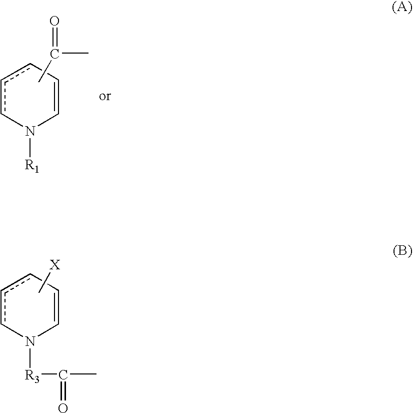
wherein the dotted line indicates the presence of a double bond in either the 4- or 5-position of the dihydropyridine ring; R₁ is C₁-C₇ alkyl or C₇-C₁₀ aralkyl; R₃ is C₁-C₃ alkylene; X is —CONR′R″ wherein each of R′ and R″, which are the same or different, is H or C₁-C₇ alkyl, or X is —COOR′″ wherein R′″ is C₁-C₇ alkyl or C₇-C₁₀ aralkyl; the carbonyl grouping in (A) is attached at the 2-, 3- or 4-position of the dihydropyridine ring; and the X grouping in (B) is attached at the 2-, 3- or 4-position of the dihydropyridine ring;
(b) D is the residue of an anti-inflammatory steroid having at least one reactive hydroxyl functional group, one such hydroxyl group being a 21-hydroxy substituent, said residue being characterized by the absence of a hydrogen atom from at least one of the reactive hydroxyl functional groups; and n and [DHC] are defined as above; or
(c) D is the residue of a steroidal androgen having a reactive 17β-hydroxyl functional group, said residue having a hydrogen atom absent from the 17β-hydroxyl functional group; n is one and [DHC] is defined as above;
the amount of S-CDS in the complex being an amount effective to elicit a therapeutic response while maintaining acceptably low peripheral steroid levels.

58. Use of a substantially saturated complex of the compound 17β-[(1-methyl-1,4-dihydro-3-pyridinyl)carbonyloxy]estra-1,3,5(10)-trien-3-ol with a hydroxyalkyl, carboxyalkyl or carboxymethylethyl derivative of β- and γ-cyclodextrin in the preparation of a buccal tablet, buccal wafer or buccal patch comprising an anhydrous formulation of said substantially saturated complex comprising from about 0.01 to about 2.0 mg of said compound, for enhancing the transmucosal bioavailability of said compound.

59. A method for the treatment of symptoms of a condition responsive to a steroidal estrogen, a steroidal progestin, an anti-inflammatory steroid or a steroidal androgen in a subject in need of such treatment, said method comprising transmucosally administering to said subject an effective estrogenic, progestational, anti-inflammatory or androgenic amount, respectively, of a pharmaceutical composition comprising an essentially saturated S-CDS-cyclodextrin complex formulated into a transmucosal dosage form, said composition being substantially free of cyclodextrin in excess of the minimum amount required to maximize the amount of S-CDS in the complex, said S-CDS being a compound of the formula:
DDHC]ₙ (I)
or a non-toxic pharmaceutically acceptable salt thereof, wherein:
(a) D is the residue of a steroidal estrogen or steroidal progestin having one or two reactive hydroxyl functional groups, one such hydroxyl group being a 17β-hydroxy substituent, said residue having a hydrogen atom absent from at least one of the reactive hydroxyl functional groups; n is a positive integer equal to the number of said functional groups from which a hydrogen atom is absent; and [DHC] is a radical of the formula 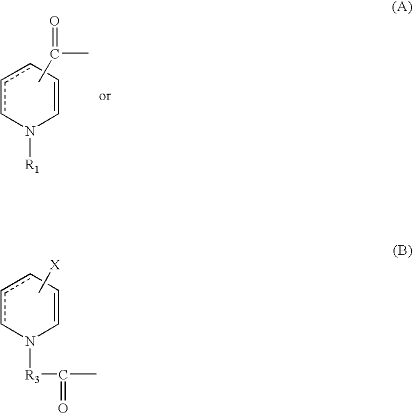
wherein the dotted line indicates the presence of a double bond in either the 4- or 5-position of the dihydropyridine ring; R₁ is C₁-C₇ alkyl or C₇-C₁₀ aralkyl; R₃ is C₁-C₃ alkylene; X is —CONR′R″ wherein each of R′ and R″, which are the same or different, is H or C₁-C₇ alkyl, or X is —COOR′″ wherein R′″ is C₁-C₇ alkyl or C₇-C₁₀ aralkyl; the carbonyl grouping in (A) is attached at the 2-, 3- or 4-position of the dihydropyridine ring; and the X grouping in (B) is attached at the 2-, 3- or 4-position of the dihydropyridine ring;
(b) D is the residue of an anti-inflammatory steroid having at least one reactive hydroxyl functional group, one such hydroxyl group being a 21-hydroxy substituent, said residue being characterized by the absence of a hydrogen atom from at least one of the reactive hydroxyl functional groups; and n and [DHC] are defined as above; or
(c) D is the residue of a steroidal androgen having a reactive 17β-hydroxyl functional group, said residue having a hydrogen atom absent from the 17β-hydroxyl functional group; n is one and [DHC] is defined as above;
the amount of S-CDS in the complex being an amount effective to elicit a therapeutic response while maintaining acceptably low peripheral steroid levels.

60. A method for the treatment of symptoms of a condition responsive to a steroidal estrogen, a steroidal progestin, an anti-inflammatory steroid or a steroidal androgen in a subject in need of such treatment, said method comprising transmucosally administering to said subject an effective estrogenic, progestational, anti-inflammatory or androgenic amount, respectively, of a pharmaceutical composition comprising an essentially saturated S-CDS-cyclodextrin complex formulated into a transmucosal dosage form, said composition being substantially free of cyclodextrin in excess of the minimum amount required to maintain substantially all of the S-CDS in the complex, said S-CDS being a compound of the formula:
DDHC]ₙ (I)
or a non-toxic pharmaceutically acceptable salt thereof, wherein:
(a) D is the residue of a steroidal estrogen or steroidal progestin having one or two reactive hydroxyl functional groups, one such hydroxyl group being a 17β-hydroxy substituent, said residue having a hydrogen atom absent from at least one of the reactive hydroxyl functional groups; n is a positive integer equal to the number of said functional groups from which a hydrogen atom is absent; and [DHC] is a radical of the formula 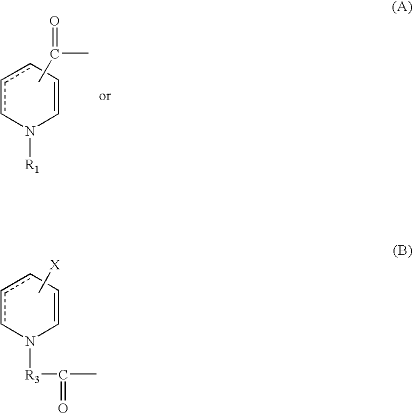
wherein the dotted line indicates the presence of a double bond in either the 4- or 5-position of the dihydropyridine ring; R₁ is C₁-C₇ alkyl or C₇-C₁₀ aralkyl; R₃ is C₁-C₃ alkylene; X is —CONR′R″ wherein each of R′ and R″, which are the same or different, is H or C₁-C₇ alkyl, or X is —COOR′″ wherein R′″ is C₁-C₇ alkyl or C₇-C₁₀ aralkyl; the carbonyl grouping in (A) is attached at the 2-, 3- or 4-position of the dihydropyridine ring; and the X grouping in (B) is attached at the 2-, 3- or 4-position of the dihydropyridine ring;
(b) D is the residue of an anti-inflammatory steroid having at least one reactive hydroxyl functional group, one such hydroxyl group being a 21-hydroxy substituent, said residue being characterized by the absence of a hydrogen atom from at least one of the reactive hydroxyl functional groups; and n and [DHC] are defined as above; or
(c) D is the residue of a steroidal androgen having a reactive 17β-hydroxyl functional group, said residue having a hydrogen atom absent from the 17β-hydroxyl functional group; n is one and [DHC] is defined as above;
the amount of S-CDS in the complex being an amount effective to elicit a therapeutic response while maintaining acceptably low peripheral steroid levels.

61. A method for the treatment of symptoms of a condition responsive to a steroidal estrogen in a woman in need of such treatment, said method comprising buccally administering to said woman a buccal tablet, buccal wafer or buccal patch comprising an anhydrous formulation of a substantially saturated complex of the compound 17β-[(1-methyl-1,4-dihydro-3-pyridinyl)carbonyloxy]estra-1,3,5(10)-trien-3-ol with a hydroxyalkyl, carboxyalkyl or carboxymethylethyl derivative of β- or γ-cyclodextrin comprising from about 0.5 to about 2.0 mg of said compound.

62. A method for the treatment of symptoms of a condition responsive to a steroidal estrogen in a man in need of such treatment, said method comprising buccally administering to said man a buccal tablet, buccal wafer or buccal patch comprising an anhydrous formulation of a substantially saturated complex of the compound 17β-[(1-methyl-1,4-dihydro-3-pyridinyl)carbonyloxy]estra-1,3,5(10)-trien-3-ol with a hydroxyalkyl, carboxyalkyl or carboxymethylethyl derivative of β- or γ-cyclodextrin comprising from about 0.01 up to but not including 0.5 mg of said compound.

63. Use of an essentially saturated S-CDS-cyclodextrin complex in the formulation of a transmucosal dosage form substantially free of cyclodextrin in excess of the minimum amount required to maximize the amount of S-CDS in the complex, for administration in the treatment of symptoms of a condition responsive to a steroidal estrogen, a steroidal progestin, an anti-inflammatory steroid or a steroidal androgen, said S-CDS being a compound of the formula:
DDHC]ₙ (I)
or a non-toxic pharmaceutically acceptable salt thereof, wherein:
(a) D is the residue of a steroidal estrogen or steroidal progestin having one or two reactive hydroxyl functional groups, one such hydroxyl group being a 17β-hydroxy substituent, said residue having a hydrogen atom absent from at least one of the reactive hydroxyl functional groups; n is a positive integer equal to the number of said functional groups from which a hydrogen atom is absent; and [DHC] is a radical of the formula 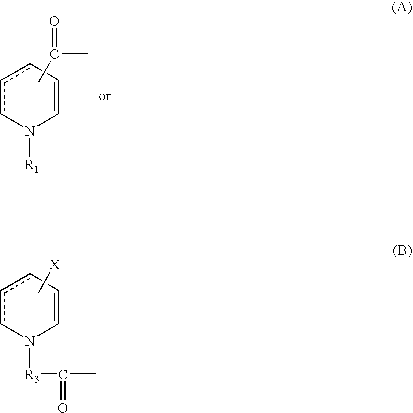
wherein the dotted line indicates the presence of a double bond in either the 4- or 5-position of the dihydropyridine ring; R₁ is C₁-C₇ alkyl or C₇-C₁₀ aralkyl; R₃ is C₁-C₃ alkylene; X is —CONR′R″ wherein each of R′ and R″, which are the same or different, is H or C₁-C₇ alkyl, or X is —COOR′″ wherein R′″ is C₁-C₇ alkyl or C₇-C₁₀ aralkyl; the carbonyl grouping in (A) is attached at the 2-, 3- or 4-position of the dihydropyridine ring; and the X grouping in (B) is attached at the 2-, 3- or 4-position of the dihydropyridine ring;
(b) D is the residue of an anti-inflammatory steroid having at least one reactive hydroxyl functional group, one such hydroxyl group being a 21-hydroxy substituent, said residue being characterized by the absence of a hydrogen atom from at least one of the reactive hydroxyl functional groups; and n and [DHC] are defined as above; or
(c) D is the residue of a steroidal androgen having a reactive 17β-hydroxyl functional group, said residue having a hydrogen atom absent from the 17β-hydroxyl functional group; n is one and [DHC] is defined as above;
the amount of S-CDS in the complex being an amount effective to elicit a therapeutic response while maintaining acceptably low peripheral steroid levels.

64. Use of an essentially saturated S-CDS-cyclodextrin complex in the formulation of a transmucosal dosage form substantially free of cyclodextrin in excess of the minimum amount required to maintain substantially all of the S-CDS in the complex, for administration in the treatment of symptoms of a condition responsive to a steroidal estrogen, a steroidal progestin, an anti-inflammatory steroid or a steroidal androgen, said S-CDS being a compound of the formula:
DDHC]ₙ (I)
or a non-toxic pharmaceutically acceptable salt thereof, wherein:
(a) D is the residue of a steroidal estrogen or steroidal progestin having one or two reactive hydroxyl functional groups, one such hydroxyl group being a 17β-hydroxy substituent, said residue having a hydrogen atom absent from at least one of the reactive hydroxyl functional groups; n is a positive integer equal to the number of said functional groups from which a hydrogen atom is absent; and [DHC] is a radical of the formula 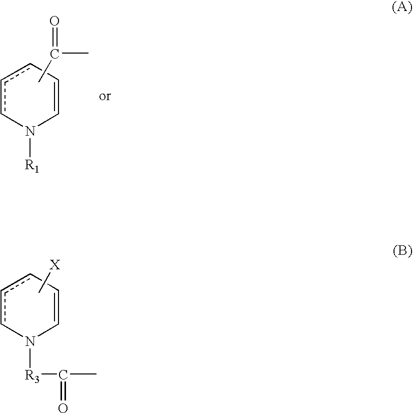
wherein the dotted line indicates the presence of a double bond in either the 4- or 5-position of the dihydropyridine ring; R₁ is C₁-C₇ alkyl or C₇-C₁₀ aralkyl; R₃ is C₁-C₃ alkylene; X is —CONR′R″ wherein each of R′ and R″, which are the same or different, is H or C₁-C₇ alkyl, or X is —COOR′″ wherein R′″ is C₁-C₇ alkyl or C₇-C₁₀ aralkyl; the carbonyl grouping in (A) is attached at the 2-, 3- or 4-position of the dihydropyridine ring; and the X grouping in (B) is attached at the 2-, 3- or 4-position of the dihydropyridine ring;
(b) D is the residue of an anti-inflammatory steroid having at least one reactive hydroxyl functional group, one such hydroxyl group being a 21-hydroxy substituent, said residue being characterized by the absence of a hydrogen atom from at least one of the reactive hydroxyl functional groups; and n and [DHC] are defined as above; or
(c) D is the residue of a steroidal androgen having a reactive 17β-hydroxyl functional group, said residue having a hydrogen atom absent from the 17β-hydroxyl functional group; n is one and [DHC] is defined as above;
the amount of S-CDS in the complex being an amount effective to elicit a therapeutic response while maintaining acceptably low peripheral steroid levels.

65. Use of a substantially saturated complex of the compound 17β-[(1-methyl-1,4-dihydro-3-pyridinyl)carbonyloxy]estra-1,3,5(10)-trien-3-ol with a hydroxyalkyl, carboxyalkyl or carboxymethylethyl derivative of β- or γ-cyclodextrin, in the preparation of a buccal tablet, buccal wafer or buccal patch comprising an anhydrous formulation of said substantially saturated complex comprising from about 0.01 to about 2.0 mg of said compound, for administration in the treatment of symptoms of a condition responsive to a steroidal estrogen.
